(19) United States
(12) Patent Application Publication
(10) Pub. No.: US 2023/0381343 A1
(43) Pub. Date: Nov. 30, 2023

(54) Treatment for Intraocular Pressure Related Disorders

(71) Applicant: Mayo Foundation for Medical Education and Research, Rochester, MN (US)

(72) Inventors: Michael P. Fautsch, Rochester, MN (US); Gavin W. Roddy, Rochester, MN (US); Lauren A. Dalvin, Philadelphia, PA (US)

(73) Assignee: Mayo Foundation for Medical Education and Research, Rochester, MN (US)

(21) Appl. No.: 18/343,437

(22) Filed: Jun. 28, 2023

Related U.S. Application Data
(63) Continuation of application No. PCT/US2022/011344, filed on Jan. 5, 2022.
(60) Provisional application No. 63/214,666, filed on Jun. 24, 2021.
(60) Provisional application No. 63/133,947, filed on Jan. 5, 2021.

(51) Int. Cl. A61K 48/00; A61K 9/00; A61K 38/22; A61P 27/06

(52) U.S. Cl. CPC A61K 48/0058; A61K 48/0066; A61K 48/0041; A61P 27/06; A61K 48/0075; A61K 38/22; A61K 9/0048

(57) ABSTRACT

Provided herein are compositions and methods for reducing IOP in a patient suffering from an IOP-related ocular disorder or an ocular disorder responsive to the lowering of IOP, the compositions comprising a nucleic acid encoding the hormone peptide human stanniocalcin-1 (STC-1). In aspects provided herein, the STC-1 encoding nucleic acid is contained in a recombinant adeno-associated virus (rAAV) vector comprising an AAV capsid comprising a nucleic acid packaged therein, wherein the nucleic acid comprises an AAV 5′ inverted terminal repeat (ITR), a promoter, and a coding sequence encoding a STC-1 polypeptide, and an AAV 3′ ITR (rAAV-STC-1). The rAAV-STC-1 compositions and methods provided herein allow for repeatable, diffuse, and sustained expression of STC-1 for extended periods, resulting in prolonged periods of IOP-reduction in patients most at need.

### CROSS REFERENCE TO RELATED APPLICATIONS

This application is a continuation of International Patent Application No. PCT/US2022/011344, filed Jan. 5, 2022, which claims benefit of U.S. Provisional Application No. 63/133,947, filed Jan. 5, 2021, and U.S. Provisional Application No. 63/214,666, filed Jun. 24, 2021. The entirety of each of these applications is hereby incorporated by reference herein for all purposes.

### FIELD OF THE INVENTION

The present invention relates to methods and compositions for treating a patient having an ocular disorder related to intraocular pressure (IOP) such as normal-tension glaucoma (NTG), glaucomatous optic neuropathy (GON), or radiation papillopathy.

### INCORPORATION BY REFERENCE

The contents of the XML file named “19081-013WO1_ST26_2023-06-22”, which was created on Jun. 22, 2023, and is 90.4 KB in size, are hereby incorporated by reference in their entirety.

### BACKGROUND OF THE INVENTION

Neuropathy associated with glaucoma (glaucomatous optic neuropathy (GON)) remains the world's leading cause of irreversible blindness, estimated to effect 80 million people worldwide, and is a diagnosis that refers to a specific pattern of progressive retinal ganglion cell loss that is pressure-sensitive in nature (Quigley et al., Br. J. Ophthalmol., 90(3):262-267 (2006)). GON is broadly divided into open-angle and closed-angle glaucoma each with primary and secondary causes. For all of the glaucomas, the only reliable therapeutic target is the reduction of IOP, the most prevalent risk factor for GON. Ocular hypertension, or elevated IOP in the absence of GON may also be treated with IOP-lowering medications.

Neuropathy also occurs in normal-tension glaucoma (NTG), also known as low tension or normal pressure glaucoma, which is a form of glaucoma in which damage occurs to the optic nerve without eye pressure exceeding the normal range. In general, a “normal” pressure range is between about 12-21 mm Hg. While the causes of NTG are still unknown, the optic nerve is susceptible to damage from even the normal amount of eye pressure. In treating NTG, it is desirable to reduce the patient's IOP as low as possible, including through the administration of IOP-lowering medications, laser treatments, and conventional surgery.

IOP is a function of ocular aqueous humor production and drainage. Aqueous humor drainage occurs via two major pathways (Costagliola et al. Surv Ophthalmol 65, 144-170 (2020)). The conventional, or trabecular pathway is the predominant pathway that drains the majority of aqueous through the trabecular meshwork into a venous plexus in the episcleral venous system which has a basal pressure known as episcleral venous pressure (EVP). The second major pathway is known as the uveoscleral outflow pathway and drains fluid primarily through the ciliary muscle. Treatment strategies for IOP reduction include pharmacologic or laser therapies that try to reduce the production or increase the drainage of aqueous. If these modalities are not sufficient, surgical intervention may be required to create a new drainage channel. Use of topical eye drops that contain ocular hypotensive therapeutics is often the initial treatment due to risks associated with laser or surgical intervention (Linden, C. et al. Acta Ophthalmol 96, 567-572 (2018)).

Generally, topical prostaglandin F2 alpha (PGF2α) analogue eye drop monotherapy (e.g., latanoprost, bimatoprost) is the initial treatment of choice for patients with GON or ocular hypertension (Linden et al., Acta Ophthalmol., 96(6):567-572 (2018)), and is a treatment option for those with NTG. PGF2α analogues are pro-inflammatory molecules that indirectly lower IOP by signaling through the prostaglandin F (FP) receptor (Doucette et al., Ophthalmic Genet., 38(2): 108-16(2017)).

There exist several problematic features of PGF2α eye drops, however, which involve the method of application, side-effects, and overall compliance. For example, latanoprost eye drops (Xalatan®, NDA 20-597/S-044) must be applied once per day and any deviation in dosage may not only decrease the IOP lowering effect but paradoxically lead to elevations in IOP. The requirement of daily administration contributes to substantial non-compliance in adherence to the daily regimen, with estimates of non-compliant patients ranging between 23-60% of all glaucoma patients (Richardson et al., Patient Preference and Adherence, 7:1025-1039(2013); Gooch et al., Pharmaceutics, 4:197-211(2012); Mansberger et al., Trans Am Ophthalmol Soc. 111:1-16(2013); Budenz et al., Ophthalmology 116:S43-7(2009); Sleath et al., Ophthalmology 118:2398-2402(2011)).

Another factor that also leads to poor regimen adherence is the collection of side effects associated with latanoprost and other PGF2α analogue eye drops. Though generally well-tolerated, latanoprost and other PGF2α analogues have notable inflammation-related side effects including conjunctival hyperemia, punctate epithelial keratopathy, and ocular surface and intraocular irritation (Toris et al., Surv Ophthalmol., 53 Suppl1:S107-120(2008); Smith et al., Acta Ophthalmol Scand., 77(6):668-72; Warwar et al., Ophthalmology, 105(2):263-8; Razeghinejad et al., Ocul Immunol Inflamm. 2017:1-8(2017)). Poor compliance due to negative side-effects or the difficulty of daily administration unfortunately results in increased disease progression (Rajurkar et al., J Curr Ophthalmol, 30:125-9(2018); Newman-Casey et al., Ophthalmology, 122:1308-16(2015); Nordstrom et al., Am J Ophthalmol, 140:598-606(2005); Hwang et al., JAMA Ophthalmol, 132:1446-52(2014); Feehan et al., J Clin Med, 5(2016)).

Despite the wide application of PGF2α analogues, a substantial segment (˜20%) of patients with ocular hypertension, GON, and NTG have diminished or absent responses to PGF2α analogue eye drops such as latanoprost (Doucette et al., Ophthalmic Genet., 38(2):108-116 (2017); King et al., BMJ, 346:f3518 (2013); Tanna et al., Curr. Opin. Ophthalmol., 26:116-120 (2015); Winkler et al., J. Ocul. Pharmacol. Therap., 30(2-3):102-109 (2014); Cui et al., J. Clin. Pharm. Ther., 42(1):87-92(2017); Zhang et al., Curr. Eye Res., 41(12):1561-1565 (2016); Ussa et al., Ophthalmology, 122(5):1040-1048 e1044 (2015); Sakurai et al., Br. J. Ophthalmol., 98(4):469-473 (2014); Sakurai et al., Ophthalmology, 114(6):1039-1045 (2007); Peng et al., Kobe J. Med. Sci., 53(1-2): 49-52 (2007)). This substandard response to PGF2α analogue eyedrops has been associated with genetic polymorphisms in the FP receptor gene (PTGFR) (Cui et al., J Clin Pharm Ther., 42(1):d87-92(2017); Zhang et al., Curr Eye Res., 41(12):1561-5(2016); Ussa et al., Ophthalmology, 122(5):1040-8(2015); Sakurai et al., Br J Ophthalmol, 98(4):469-73(2014); Sakurai et al., Ophthalmology, 114(6):1039-45(2007); Peng et al., Kobe J Med Sci., 53(1-2):49-52(2007)).

In order to combat these issues, sustained IOP reduction is an increasingly sought-after therapeutic goal in order to minimize patient non-compliance, ocular surface side effects, and IOP fluctuation. A variety of approaches to deliver available medications have been employed, including contact lens delivery and implants to the conjunctival fornix, lacrimal puncta, periocular space, or anterior chamber (see recent review by Kompella et al. Prog Retin Eye Res, 100901 (2020)). Available approaches, however, require the use of a scaffold to elute drug which may lead to patient discomfort when used extraocularly or complications including corneal endothelial cell loss when used intraocularly.

In addition to the ocular hypertensive neuropathies described above, additional neuropathies have been associated with, for example, radiation-based ocular therapies, including plaque, proton beam, and gamma knife treatments for ocular tumors, for example, uveal melanoma (Jampol et al. Ophthalmol 109:2197-2206(2002); Papakostas et al. JAMA Ophthalmol. 135:1191-6(2017); Lane et al. JAMA Ophthalmol. 133:792-796(2015); Gragoudas et al. Trans Am Ophthalmol Soc. 100:43-9(2002); Gorovets et al. Brachytherapy 16:433-43(2017); Reynolds et al. Int J Retin Vitreous 3:17(2017)). In certain patients, radiation side effects limit long-term visual acuity, resulting in radiation papillopathy (see, e.g., Kim et al. Natural history of radiation papillopathy after proton beam irradiation of parapapillary melanoma, Ophthalmology 2010; 117;1617-1622). Radiation dose to the optic disc is a well-established risk factor for postradiation optic atrophy and is associated with a poor visual acuity outcome. Over the course of several years, some cases of postradiation optic atrophy display optic disc pallor as the predominant feature, whereas others develop concomitant neuroretinal rim thinning (NRT). Preventing and treating these radiation papillopathies remains a challenge.

Accordingly, there is an unmet need for compositions and methods useful for treating ocular neuropathies in patients in need thereof without the requisite issues associated with current modalities.

### SUMMARY OF THE INVENTION

Provided herein are compositions, and methods for using such compositions, to reduce IOP in a patient suffering from an IOP-related ocular disorder or an ocular disorder responsive to the lowering of IOP, the compositions comprising a nucleic acid encoding the hormone peptide human stanniocalcin-1 (STC-1). In aspects provided herein, the STC-1 encoding nucleic acid is contained in a recombinant adeno-associated virus (rAAV) vector comprising an AAV capsid comprising a nucleic acid packaged therein, wherein the nucleic acid comprises an AAV 5′ inverted terminal repeat (ITR), a promoter, and a coding sequence encoding a STC-1 polypeptide, and an AAV 3′ ITR (rAAV-STC-1). The rAAV-STC-1 compositions and methods provided herein allow for repeatable, diffuse, and sustained expression of STC-1 for extended periods, resulting in prolonged periods of IOP-reduction in patients most at need.

The methods and compositions provided herein can be used to treat or prevent an ocular disorder in a patient associated with increased or elevated IOP, for example GON or ocular hypertension. In addition, the methods and compositions provided herein can be used to treat or prevent an ocular disorder in a patient wherein the disorder is responsive to a reduction of IOP, for example, NTG. For example, administration of STC-1 to the eye is capable of lowering IOP in eyes with hypertension and lowering IOP from normal baseline tension levels (see, e.g., Example 6, FIG. 23B). Furthermore, the methods and compositions provided herein can be used to treat those at risk for developing radiation papillopathy, for example, individuals with elevated baseline IOP at the time of receiving radiation to treat an ocular tumor, or who develop an elevated IOP after receiving radiation to treat an ocular tumor. In some embodiments, the rAAV-STC-1 compositions and methods provided herein can be used to repeatedly reduce IOP and/or prevent elevated IOP in one or more eyes of a patient without the need for implanting a drug-eluting device into the lacrimal puncta, ocular surface, or anterior chamber of the patient. In some embodiments, the patient's IOP is non-responsive to latanoprost or other PGF2α analogues.

Human STC-1 (UniProtKB-P52823 (STC1_HUMAN)), a 50-kDa disulfide-linked dimer with 11 paired cysteine residues, has been shown to function in mineral metabolism and contains anti-inflammatory (Huang et al. Am J Pathol 174, 1368-1378 (2009)), anti-oxidative stress (Sheikh-Hamad, D. Am J Physiol Renal Physiol 298, F248-254 (2010)) and neuroprotective (Roddy et al. Mol Ther 20, 788-797 (2012)) properties. STC-1 functions as a stress-response protein being produced at relatively low levels during periods of physiologic homeostasis, but upregulated in times of cellular stress (Dalvin et al. Curr Eye Res, 1-6 (2019)), including inflammation (Tang et al. Free Radic Biol Med 71, 321-331 (2014), oxidation (Nguyen et al., Oncogene 28, 1982-1992 (2009)), and hypoxia (Ito et al. Biochemical and biophysical research communications 452, 1091-1097 (2014); Shi et al., J Cardiovasc Pharmacol 64, 522-529 (2014); Durukan Tolvanen et al., Neuroscience 229, 49-54, (2013)).

Advantageously, a rAAV-STC-1 composition provided herein can be repeatedly administered, as needed. For example, a rAAV-STC-1 composition can be repeatedly administered to, for example, the anterior chamber of the eye via intracameral injection or via subconjunctival delivery or other suitable delivery area, without the induction of a disadvantageous and further compounding inflammatory response. Surprisingly, it is believed that the expression of STC-1 itself may contribute to the tolerability of repeat injections of the present invention compositions due to its anti-inflammatory properties, possibly through the inhibition of macrophage chemotaxis, modulation of transendothelial migration of leukocytes, and reduction of T-cell infiltration. Thus STC-1 unexpectedly serves a dual purpose: i) the induction of IOP reduction to treat an ocular disorder, and ii) an anti-inflammatory adjunctive therapy that unexpectedly assists with host tolerability of repeat viral vector administrations. The ability to repeatedly administer an AAV vector comprising a nucleic acid encoding STC-1 as described herein is believed to represent the first successful repeat delivery of an AAV viral vector into the anterior chamber or subconjunctival space to achieve a therapeutic response for the reduction of IOP. For example, and as described further below (see, e.g., Example 2 and FIG. 6A-C, 7A-B) a repeat injection of a single strand (ss) AAV2 comprising a nucleic acid encoding STC-1 via a constitutively active promoter (chicken β-actin) (ssAAV2-smCBA-STC-1-FLAG) intracamerally into the anterior chamber leads to the reestablishment of IOP reduction in relevant glaucoma animal models. Likewise, a repeat injection of a ssAAV2-smCBA-STC-1-FLAG to the subconjunctival space also leads to the reestablishment of IOP reduction in relevant glaucoma animal models (see, e.g., Example 2, FIG. 11).

The ability to repeatedly administer the recombinant viral vectors of the present invention represents a significant advance in IOP-reducing treatments, as a major concern with the therapeutic use of viral constructs including AAV for the repeated delivery of therapeutic agents is the potential to induce an inflammatory response with initial or repeat injections that may limit host tolerability of the treatment (Riviere et al., Gene Ther, 13:1300-08(2006)); Mingozzi et al., Blood, 122:23-36(2013); Perez et al., Brain Sci., 10, (2020)). For example, a single intravitreal injection into vitreous humor of an AAV engineered to express green fluorescent protein was shown to produce intraocular inflammation characterized by a CD45+ inflammatory infiltrate in the vitreous (Goel et al., Open Ophthalmol, 4:52-59(2010)).

In one aspect, these advantageous effects are derived via delivery of a rAAV-STC-1 composition to the anterior chamber of one or both eyes. The anterior chamber contains aqueous humor, a water-like substance produced and secreted from the ciliary epithelium that nourishes the tissues in the front of the eye and is removed through several drainage pathways. This continuous production and removal of aqueous humor results in turnover of aqueous humor every 90-120 minutes (see, e.g., Bakri et al., Ophthalmology 114, 855-859 (2007)), minimizing viral exposure while allowing for diffuse expression and delivery into the natural outflow pathway of the eye, allowing for the delivery of STC-1 to, for example, the iridocorneal angle. Following administration of ssAAV2-smCBA-STC-1-FLAG into the anterior chamber, expression of STC-1-FLAG was observed in the ciliary body, cornea, iris, and lens (see, e.g., FIG. 5A-B, 9, 10A-B). Unexpectedly, anterior chamber delivery also resulted in STC-1-FLAG expression and/or delivery to the retina (see, e.g., FIG. 5A-B, 9, 10A-B), demonstrating robust in situ retinal expression observed more than 4 months post-administration. The ability to express/deliver STC-1 in the retina has important implications for direct neuroprotective effects of retinal ganglion cells. Because GON and NTG are primarily an optic neuropathy by definition, the use of an AAV to deliver a nucleic acid capable of constitutively expressing STC-1 in the anterior chamber of the eye provides a unique, first-in-class, dual mechanism therapeutic that provides both IOP reduction and neuroprotection. Furthermore, STC-1 acts downstream of the FP-receptor, and the effects of STC-1 are not dependent on the FP-receptor for activity, unlike PGF2α analogues such as latanoprost (Roddy et al., Invest Ophthalmol Vis Sci. 58(5):2715-24(2017); Roddy et al., PLoS One 15(5):1-12(2020)). Accordingly, the use of a rAAV comprising a nucleic acid capable of expressing STC-1 provides better therapeutic benefit to the substantial segment (˜20%) of glaucoma patients with poor or absent responses to PGF2α analogues due to FP-receptor heterogeneity.

In an alternative aspect, these advantageous effects are derived via delivery of a rAAV-STC-1 composition described herein to the subconjunctival space of one or both eyes. As shown in Example 2, repeat subconjunctival injections of the compositions described herein reestablishes IOP reduction after the initial effect of a first administration begin to wane (FIG. 11). Importantly, the ability to administer multiple doses of the present compositions to the subconjunctival space provides a significantly less invasive administration route, eliminating many of the risks associated with intraocular delivery, including for example bleeding in the eye (hyphema) and/or infection (endophthalmitis), while allowing delivery of STC-1 into the anterior chamber, which benefits are described above.

In additional alternative aspects, a rAAV-STC-1 composition described herein can be repeatedly delivered to the eye via, for example, but not limited to, intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection.

In some embodiments, following administration of the rAAV-STC-1 compositions provided herein, IOP in a treated eye of a patient is reduced from a baseline pre-administration level by at least 5%, 10%, 15%, 20%, 25%, 30%, 35%, 40%, 45%, 50%, 55%, or greater. In some embodiments, following administration of the rAAV-STC-1 compositions provided herein, IOP in a treated eye of a patient is reduced from a pre-administration level by between about 10% and 75%, between about 10% and 65%, between about 10% and 50%, or between about 10% and 40%. In some embodiments, following administration of a rAAV-STC-1 composition described herein, IOP normalizes between about 10 mmHg and 21 mmHg for at least 30 days, at least 60 days, at least 90 days, at least 180 days, at least 1 year, at least 2 years, or longer. In some embodiments, following administration of the rAAV-STC-1 compositions provided herein, IOP in a treated eye of a patient is reduced from a baseline pre-administration level by at least 1 mmHg, at least 2 mmHg, at least 3 mmHg, at least 4 mmHg, at least 5 mmHg, at least 6 mmHg, at least 7 mmHg, at least 8, mm Hg, at least 9 mmHg, at least 10 mmHg, or greater than 10 mmHg. In some embodiments, IOP is determined using Goldmann Applanation Tonometry (GAT).

In one aspect, provided herein is a method for reducing IOP in a patient suffering from an IOP-related ocular disorder or an ocular disorder responsive to the lowering of IOP, wherein the method comprises administering to at least one eye of the patient a therapeutically effective amount of an adeno-associated virus (rAAV) comprising a nucleic acid encoding a stanniocalcin-1 (STC-1) polypeptide operably linked to a promoter. In some embodiments, the STC-1 has been codon optimized for expression from an AAV vector. In some embodiments, the STC-1 has been codon optimized for expression in a human ocular cell. Such STC-1 sequences may share at least 70%, at least 75%, at least 80%, at least 90%, at least 95%, at least 96%, at least 97%, at least 98%, or at least 99% identity to the wild-type or native human STC-1 coding sequence (SEQ ID NO:1)(NCBI CCDS 6043.1). In some embodiments, the nucleic acid encodes an STC-1 polypeptide having the amino acid sequence of SEQ ID NO:2, or an amino acid at least 70%, at least 75%, at least 80%, at least 90%, at least 95%, at least 96%, at least 97%, at least 98%, or at least 99% identical thereto. In some embodiments, the nucleic acid encodes an STC-1 polypeptide having the amino acid sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 20; SEQ ID NO: 21, SEQ ID NO:22, SEQ ID NO:23, SEQ ID NO:25, SEQ ID NO: 27, or SEQ ID NO: 29, or an amino acid at least 70%, at least 75%, at least 80%, at least 90%, at least 95%, at least 96%, at least 97%, at least 98%, or at least 99% identical thereto. In some embodiments, the nucleic acid encoding STC-1 is SEQ ID NO:1, or a nucleic acid having at least 70%, at least 75%, at least 80%, at least 90%, at least 95%, at least 96%, at least 97%, at least 98%, or at least 99% identical thereto. In some embodiments, the nucleic acid encoding STC-1 is selected from SEQ ID NO:3, SEQ ID NO:5, SEQ ID NO:7, SEQ ID NO:9, SEQ ID NO: 11, SEQ ID NO: 13, SEQ ID NO: 15, SEQ ID NO:17, SEQ ID NO:19, SEQ ID NO:24, SEQ ID NO:26, or SEQ ID NO:28, or a nucleic at least 70%, at least 75%, at least 80%, at least 90%, at least 95%, at least 96%, at least 97%, at least 98%, or at least 99% identical thereto.

In some embodiments, the promoter operably linked to the nucleic acid encoding STC-1 is a constitutively active promoter. In some embodiments, the constitutively active promoter is derived from, for example but not limited to a cytomegalovirus (CMV) promoter, a beta actin promoter, for example but not limited to a chicken beta actin (CBA) promoter or a human beta actin (hACTB) promoter, cytomegalovirus (CMV) immediate-early enhancer and chicken beta-actin (CAG) promoter, a human elongation factor-1 alpha (hEF-1α) promoter, a phosphoglycerate kinase (PGK) promoter, a ubiquitin C (UbiC) promoter, or other constitutively active promoter described herein. In some embodiments, the promoter is a CBA promoter derived from the nucleic acid sequence of SEQ ID NO:33, SEQ ID NO:34, or SEQ ID NO:35, or a nucleic acid sequence at least 70%, at least 75%, at least 80%, at least 90%, at least 95%, at least 96%, at least 97%, at least 98%, or at least 99% identical thereto. In some embodiments, the CBA promoter is SEQ ID NO:34, or a nucleic acid at least 70%, at least 75%, at least 80%, at least 90%, at least 95%, at least 96%, at least 97%, at least 98%, or at least 99% identical thereto. In some embodiments, the CBA promoter is SEQ ID NO:35, or a nucleic acid at least 70%, at least 75%, at least 80%, at least 90%, at least 95%, at least 96%, at least 97%, at least 98%, or at least 99% identical thereto.

In some embodiments, the promoter is an ocular cell specific promoter. In some embodiments, the ocular cell specific promoter is derived from, for example but not limited to, human synapsin 1 gene (hSYN1) promoter, Purkinje cell protein 2 (PCP2) promoter, G Protein Subunit Gamma Transducin 2 (GNGT2) promoter, Phosphodiesterase 6H (PDE6H) promoter, Paired Like Homeodomain 3 (PITX3) promoter, claudin 5 (CLDN5) promoter, Nuclear Receptor Subfamily 2 Group E Member 1 (NR2E1) promoter, paired box 6 (PAX6) promoter, or other ocular cell specific promoter described herein. In some embodiments, the promoter is a hSYN1 promoter derived from the nucleic acid sequence of SEQ ID NO:42, or a nucleic acid sequence at least 70%, at least 75%, at least 80%, at least 90%, at least 95%, at least 96%, at least 97%, at least 98%, or at least 99% identical thereto. In some embodiments, the promoter is a hRK1 promoter derived from the nucleic acid sequence of SEQ ID NO:54, or a nucleic acid sequence at least 70%, at least 75%, at least 80%, at least 90%, at least 95%, at least 96%, at least 97%, at least 98%, or at least 99% identical thereto.

In some embodiments, the promoter is endothelial cell specific promoters. In some embodiments, the promoter is an epithelial cell specific promoter. In some embodiments, the promoter is a fibroblast cell specific promoter. In some embodiments, the endothelial cell-specific promoter is derived from, for example but not limited to, fibroblast-specific protein 1 (FSP1/S100A4) promoter, angiopoietin receptor (TEK/TIE2) promoter, vascular endothelial cadherin 5 (CDH5) promoter, vascular endothelial growth factor receptor 2 (VEGFR2/KDR/FLK1) promoter, platelet derived growth factor B (PDGFB) promoter, SRY-Box transcription factor 17 (SOX17) promoter, BMX non-receptor tyrosine kinase (BMX) promoter, endothelial cell specific molecule 1 (ESM1) promoter, apelin (APLN) promoter, apelin receptor (APLNR/APJ) promoter, fatty acid binding protein 4 (FABP4/AP2) promoter, calcium/calcineurin-dependent transcription factor (NFATC1) promoter, natriuretic peptide receptor 3 (NPR3) promoter, or other endothelial cell specific promoter described herein.

When the STC-1 encoding nucleic acid is packaged for delivery in an AAV vector, the promoter and STC-1 encoding nucleic acid are flanked by AAV inverted terminal repeats (ITRs). In some embodiments, the ITRs are derived from the same AAV serotype as the AAV capsid. In some embodiments, the ITRs are derived from a different AAV serotype than the AAV capsid. In some embodiments, the ITR is derived from an AAV2 ITR. In some embodiments, the ITR is derived from the nucleic acid of SEQ ID NO:44, SEQ ID NO:45, SEQ ID NO:46, SEQ ID NO:47, SEQ ID NO:48, SEQ ID NO:49, SEQ ID NO:56, or SEQ ID NO:57, or a nucleic acid at least 70%, at least 75%, at least 80%, at least 90%, at least 95%, at least 96%, at least 97%, at least 98%, or at least 99% identical thereto. In some embodiments, the 5′ ITR is derived from the nucleic acid of SEQ ID NO:44, SEQ ID NO:45, SEQ ID NO:46, or SEQ ID NO:56, or a nucleic acid at least 70%, at least 75%, at least 80%, at least 90%, at least 95%, at least 96%, at least 97%, at least 98%, or at least 99% identical thereto. In some embodiments, the 3′ ITR is derived from the nucleic acid of SEQ ID NO:47, SEQ ID NO:48, SEQ ID NO:49, or SEQ ID NO:57 or a nucleic acid at least 70%, at least 75%, at least 80%, at least 90%, at least 95%, at least 96%, at least 97%, at least 98%, or at least 99% identical thereto.

In some embodiments, the AAV vector is a single stranded (ss) AAV vector. In some embodiments, the AAV vector is a self-complementary (sc) AAV vector.

In certain embodiments, the nucleic acid further comprises one or more regulatory elements, enhancer elements, cis-regulatory modules (CRMs), introns, exons, polyadenylation signals, and/or post-transcriptional elements such as a Woodchuck Hepatitis Virus (WHP) Posttranscriptional Regulatory Element (WPRE).

In some embodiments, the AAV capsid is derived from AAV1, AAV2, AAV4, AAV5, AAV6, AAV8, or AAV9 serotype. In some embodiments, the AAV capsid is derived from AAV2. In some embodiments, the AAV2 is AAV2(Triple Y-F). In some embodiments, the AAV capsid is derived from AAV8. In some embodiments, the AAV capsid is derived from AAV9. In some embodiments, the AAV capsid is a hybrid of AAVs such as AAV8/AAV9 capsid, for example, AAV8G9.

In some embodiments, the rAAV comprises a nucleic acid sequence of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto. In some embodiments, the rAAV comprises a nucleic acid sequence of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto, and the AAV is an AAV2 serotype. In some embodiments, the rAAV comprises a nucleic acid sequence of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto, and the AAV is an AAV2(Triple Y-F) serotype having a tyrosine to phenylalanine mutation in its capsid protein as follows: Y444F, Y500F, and Y730F (Petrs-Silva et al. Mol Ther. 19(2):293-301(2011)). In some embodiments, the rAAV comprises a nucleic acid sequence of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto, and the AAV is an AAV8 serotype. In some embodiments, the rAAV comprises a nucleic acid sequence of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto, and the AAV is an AAV9 serotype. In some embodiments, the rAAV comprises a nucleic acid sequence of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto, and the AAV is an AAV8G9 serotype.

In some embodiments, the nucleic acid encoding STC-1 is delivered via an alternative vector, for example, but not limited to an adenoviral vector, a lentiviral vector, for example but not limited to an human-immunodeficiency virus-derived lentiviral vector or equine infectious anemia virus (EIAV)-derived lentivirus pseudotypes with VSV-G envelope protein vector, or DNA plasmid, or as an mRNA vector.

The methods and compositions described herein are particularly useful in reducing IOP in a patient, including a human, in need thereof. In some embodiments, the patient suffers from an increase in IOP, and the increase in IOP is a result of an underlying ocular disease or disorder. In some embodiments, the patient has a normal IOP, but has an ocular disorder that is responsive to a reduction in IOP, including but not limited to normal-tension glaucoma. In some embodiments, the underlying ocular disease or disorder is glaucomatous optic neuropathy (GON). In some embodiments, the underlying ocular disorder is a glaucoma. In some embodiments, the glaucoma is a primary glaucoma, for example but not limited to open angle glaucoma, normal-tension glaucoma, angle-closure glaucoma, or congenital glaucoma. In some embodiments, the glaucoma is a secondary glaucoma, for example but not limited to neovascular glaucoma, pigmentary glaucoma, exfoliation glaucoma, or uveitic glaucoma.

In an alternative aspect, the methods and compositions described herein are used to prevent or reduce the development of radiation papillopathy, which is a complication of irradiation of intraocular tumors, for example in the parapapillary area. As described in Example 5 and FIG. 15-21, it has been discovered that individuals who experience increased IOP before or during radiation treatments for an ocular tumor are at a significant risk for developing radiation papillopathy. Accordingly, in some embodiments, the compositions and methods described herein are used to treat or prevent radiation papillopathy in a patient receiving radiation for the treatment of an ocular tumor. In some embodiments, the patient is administered a composition described herein prior to initiation of the radiation. In some embodiments, the patient is administered a composition described herein two or more times during the course of radiation treatment, for example, 2, 3, 4, 6, 7, 8, 9, 10, 11, 12, 13, 14, 15, or more times during treatment. In some embodiments, the patient has an elevated IOP prior to administration of the radiation, for example, an IOP of greater than 21 mmHg. In some embodiments, the patient has an elevated IOP after administration of the radiation, for example, an IOP of greater than 21 mmHg. In some embodiments, the intraocular tumor is, for example but not limited to a melanoma, for example a choroidal melanoma or uveal melanoma, a choroidal hemangioma, a choroidal nevus, an iris tumor, a retinoblastoma, a lacrimal gland tumor, or an intraocular lymphoma. In some embodiments, the patient is suffering from radiation papillopathy following receiving radiation treatment for an ocular tumor, and is administered a composition described herein two or more times following radiation papillopathy onset, for example, 2, 3, 4, 5, 6, 7, 8, 9, 10, 11, 12, 13, 14, 15 or more administrations. In some embodiments, the patient with radiation papillopathy has optic disc pallor. In some embodiments, the patient with radiation papillopathy has optic disc pallor with concomitant neuroretinal rim thinning (NRT).

The compositions described herein are particularly suitable for multiple administrations, for example, 2, 3, 4, 5, 6, 7, 8, 9, 10, 11, 12, 13, 14, 15, or more administrations, for example, unlimited administrations, spaced over time, for example 1 week apart, 2 weeks apart, 1 month apart, 2 months apart, 3 months apart, 4 months apart, 5 months apart, 6 months apart, 7 months apart, 8 months apart, 9 months apart, 10 months apart, 11 months apart, 12 months apart, 16 months apart, 18 months apart, 24 months apart, 30 months apart, 36 months apart, 48 months apart, 60 months apart or longer. In some embodiments, the compositions described herein are administered once a week, once every two weeks, once a month, once every 2 months, once every 3 months, once every 4 months, once every 5 months, once every 6 months, once every 7 months, once every 8 months, once every 9 months, once every 10 months, once every 12 months, once every 16 months, once every 18 months, once every 24 months, once every 30 months, once every 36 months, once every 48 months, once every 60 months, or once every 10 years. In some embodiments, the composition is administered two or more times into the anterior chamber via intracameral injection. In some embodiments, the composition is administered two or more times into the subconjunctival space. In some embodiments, the composition is administered one or more times into each of the anterior chamber via intracameral injection and into the subconjunctival space. In some embodiments, the composition is administered two or more times via injection into the intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct, or a combination thereof.

In one aspect, a recombinant adeno-associated virus (rAAV) vector carrying a nucleic acid encoding STC-1 is provided for use in reducing IOP in a patient. In some embodiments, the rAAV vector comprises a nucleic acid of SEQ ID NO:50, SEQ ID NO:51, SEQ ID NO:52, or SEQ ID NO:53, or a nucleic acid at least 70%, at least 75%, at least 80%, at least 90%, at least 95%, at least 96%, at least 97%, at least 98%, or at least 99% identical thereto. In some embodiments, SEQ ID NO:50, SEQ ID NO:51, SEQ ID NO:52, or SEQ ID NO:53 is packaged into an AAV2 serotype capsid. In some embodiments, the AAV2 is a AAV2(Triple Y-F) mutant or modification thereof.

In one aspect, provided herein is an rAAV for use in reducing IOP in an eye of a patient, the rAAV comprising a AAV2 serotype capsid and a nucleic acid encoding a STC-1 polypeptide operably linked to a constitutively active promoter sequence. In some embodiments, the rAAV comprises a nucleic acid encoding an STC-1 polypeptide comprising the amino acid sequence of SEQ ID NO:2, or an amino acid sequence at least 70%, at least 75%, at least 80%, at least 90%, at least 95%, at least 96%, at least 97%, at least 98%, or at least 99% identical thereto. In some embodiments, the STC-1 polypeptide comprises the amino acid sequence of SEQ ID NO:4, or an amino acid sequence at least 70%, at least 75%, at least 80%, at least 90%, at least 95%, at least 96%, at least 97%, at least 98%, or at least 99% identical thereto. In some embodiments, the STC-1 polypeptide comprises the amino acid sequence of SEQ ID NO:6, or an amino acid sequence at least 70%, at least 75%, at least 80%, at least 90%, at least 95%, at least 96%, at least 97%, at least 98%, or at least 99% identical thereto. In some embodiments, the STC-1 polypeptide comprises the amino acid sequence of SEQ ID NO: 14, or an amino acid sequence at least 70%, at least 75%, at least 80%, at least 90%, at least 95%, at least 96%, at least 97%, at least 98%, or at least 99% identical thereto. In some embodiments, the STC-1 polypeptide comprises the amino acid sequence selected from SEQ ID NO:16, 18, 20, 21, 22, 23, 25, 27, or 29, or an amino acid sequence at least 70%, at least 75%, at least 80%, at least 90%, at least 95%, at least 96%, at least 97%, at least 98%, or at least 99% identical thereto.

In an alternative aspect, the STC-1 polypeptide further comprises an amino acid tag sequence. In some embodiments, the amino acid tag is a FLAG tag selected from the amino acid sequence of SEQ ID NO:30, SEQ ID NO:31, or SEQ ID NO:32, or an amino acid sequence at least 70%, at least 75%, at least 80%, at least 90%, at least 95%, at least 96%, at least 97%, at least 98%, or at least 99% identical thereto.

In additional alternative aspects, the rAAV comprising a nucleic acid encoding STC-1 comprises SEQ ID NO:1, or a nucleic acid sequence at least 70%, at least 75%, at least 80%, at least 90%, at least 95%, at least 96%, at least 97%, at least 98%, or at least 99% identical thereto.

In some embodiments, the nucleic acid encoding STC-1 comprises SEQ ID NO:3, or a nucleic acid sequence at least 70%, at least 75%, at least 80%, at least 90%, at least 95%, at least 96%, at least 97%, at least 98%, or at least 99% identical thereto. In some embodiments, the nucleic acid encoding STC-1 comprises SEQ ID NO:5, or a nucleic acid sequence at least 70%, at least 75%, at least 80%, at least 90%, at least 95%, at least 96%, at least 97%, at least 98%, or at least 99% identical thereto. In some embodiments, the nucleic acid encoding STC-1 comprises SEQ ID NO:13, or a nucleic acid sequence at least 70%, at least 75%, at least 80%, at least 90%, at least 95%, at least 96%, at least 97%, at least 98%, or at least 99% identical thereto. In some embodiments, the nucleic acid encoding STC-1 comprises a nucleic acid selected from SEQ ID NO:7, 9, 11, 15, 17, 19, 24, 26, or 28, or a nucleic acid sequence at least 70%, at least 75%, at least 80%, at least 90%, at least 95%, at least 96%, at least 97%, at least 98%, or at least 99% identical thereto.

In additional alternative aspects, the rAAV comprising a nucleic acid encoding STC-1 operably linked to a promoter comprises a chicken β-actin promoter. In some embodiments, the chicken β-actin promoter is derived from a nucleic acid sequence of SEQ ID NO:33, 34, or 35, or a nucleic acid sequence at least 70%, at least 75%, at least 80%, at least 90%, at least 95%, at least 96%, at least 97%, at least 98%, or at least 99% identical thereto. In some embodiments, the chicken β-actin promoter is derived from a nucleic acid sequence of SEQ ID NO:33, or a nucleic acid sequence at least 70%, at least 75%, at least 80%, at least 90%, at least 95%, at least 96%, at least 97%, at least 98%, or at least 99% identical thereto. In some embodiments, the chicken (3-actin promoter is derived from a nucleic acid sequence of SEQ ID NO:34, or a nucleic acid sequence at least 70%, at least 75%, at least 80%, at least 90%, at least 95%, at least 96%, at least 97%, at least 98%, or at least 99% identical thereto. In some embodiments, the chicken β-actin promoter is derived from a nucleic acid sequence of SEQ ID NO:35, or a nucleic acid sequence at least 70%, at least 75%, at least 80%, at least 90%, at least 95%, at least 96%, at least 97%, at least 98%, or at least 99% identical thereto. In additional alternative embodiments, the promoter is selected from a cytomegalovirus (CMV) promoter, a SV40 promoter, human β-actin promoter, a human elongation factor-1-alpha (hEF-1a) promoter, a phosphoglycerate kinase (PGK) promoter, or a ubiquitin C (UbiC) promoter.

In additional alternative aspects, the rAAV comprising a nucleic acid encoding STC-1 comprises nucleic acid SEQ ID NO:52 or SEQ ID NO:53, or a nucleic acid sequence at least 70%, at least 75%, at least 80%, at least 90%, at least 95%, at least 96%, at least 97%, at least 98%, or at least 99% identical thereto.

In additional alternative aspects, the AAV2 capsid comprises one or more tyrosine (Y) to phenylalanine (F) mutations on the surface of the capsid protein. In some embodiments, the one or more tyrosine to phenylalanine mutations on the surface of the capsid protein is selected from a Y444F mutation in its capsid protein, a Y730F mutation, a Y500F, a Y272F, a Y447F mutation, a Y733F mutation, or a Y733F mutation. In some embodiments, the AAV2 capsid comprises a tyrosine (Y) to phenylalanine (F) mutation on the surface of the capsid protein at the following amino acids: Y444F, Y500F, and Y730F.

In additional aspects, provided herein are cells comprising a nucleic acid encoding an STC-1 nucleic acid sequence as described herein.

In additional aspects, provided herein are plasmids comprising a nucleic acid encoding an STC-1 nucleic acid sequence as described herein.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A shows an exemplary construct incorporating a FLAG at the C-terminus of a human STC-1 (hSTC-1) polypeptide and maintaining the STC-1 signal peptide, which can express a hSTC-1 polypeptide under the control of a promoter, for example a chicken beta-actin constitutively active promoter. The hSTC-1 coding sequence is inserted into a plasmid containing and 3′ inverted terminal repeats (TRs) for subsequent packaging into an AAV capsid. Other construct elements may include: bovine growth hormone polyadenylation (bGH polyA) signal sequence and a simian virus 40 splice donor/splice acceptor (SV40 SD/SA) site.

FIG. 1B shows an exemplary construct incorporating a FLAG tag at the C-terminus of a hSTC-1 polypeptide and maintaining an the STC-1 signal peptide, which can express a hSTC-1 polypeptide under the control of a ocular cell specific promoter, for example the retinal ganglion cell (RGC) specific human synapsin 1 (hSYN1) promoter. The hSTC-1 coding sequence is inserted into a plasmid containing 5′ and 3′ inverted terminal repeats (TRs) for subsequent packaging into an AAV capsid. Other construct elements may include: bovine growth hormone polyadenylation (bGH polyA) signal sequence and a simian virus 40 splice donor/splice acceptor (SV40 SD/SA) site.

FIG. 2 is a diagram showing exemplary routes of ssAAV2-smCBA-STC-1-FLAG administration (arrows) to the eye. The administration routes include, but are not limited to: (1) intracameral injection into the anterior chamber (AC), (2) subretinal injection between the retina and the retinal pigmented epithelium, (3) intravitreal to the vitreous fluid, and (4) subconjunctival injection underneath the conjunctiva lining the eyelid.

FIG. 3 is a line graph showing that ssAAV2-smCBA-STC-1-FLAG reduces intraocular pressure (IOP) after intracameral injection in mice. The x-axis represents the longitudinal time length of the experiment in three phases: baseline (pre-injection) and weeks post-injection. The number of mice measured in each phase are indicated below (n=12; n=9). The y-axis represents the value of IOP in units of millimeters of mercury (mmHg). Arrows indicate the point of administration of the first and second injection in the experiment. After baseline IOP measurements, normotensive 3-month-old mice were administered intracameral injections of ssAAV-smCBA-STC-1-FLAG (▪) in one eye and PBS mock control (●) in the other eye. IOP measurements were collected longitudinally twice per week for 14 weeks post injection. IOP was reduced an average of 16.5% throughout the study period.

FIG. 4 is a bar graph showing IOP lowering response following injection with ssAAV2-smCBA-STC-1-FLAG. IOP measurements were collected longitudinally for 4 days in three-month-old C57BL/6J mice (n=26) receiving single intracameral injection of ssAAV2-smCBA-STC-1-FLAG (▪) in one eye and ssAAV2-smCBA-GFP (□) in the fellow eye. The x-axis represents the longitudinal time length of the experiment from baseline (pre-injection) to days post-injection. The number of mice measured in each phase are indicated below. Average IOP values, percent change and significance (P-value) are listed for each treatment group at each time point. The y-axis represents the value of IOP in units of mmHg. A significant IOP decrease was observed following treatment with ssAAV2-smCBA-STC-1-FLAG starting at day 2 and persisting until the end of the experiment at day 4.

FIG. 5A-FIG. 5B is a collection of micrographs of IF-stained sections showing AAV2-smCBA-STC-1-FLAG is expressed locally as well as distal to the injection site following intracameral administration. GFP was labeled with a red fluorochrome while the FLAG tag was labeled with a green fluorochrome. GFP and FLAG staining compared to secondary only controls demonstrates robust protein expression. FIG. 5A: Robust STC-1-FLAG expression in in the ciliary body (*) and evident in many tissues including the anterior segment (#), iris (\hat{ }), lens capsule (>), and the retina only one day following administration of ssAAV2-smCBA-STC-1-FLAG in three-month-old C57BL/6J mice (n=26). FIG. 5B: Prolonged AAV2 transgene expression was observed until termination of experiment at day 4 post-injection.

FIG. 6A-FIG. 6C shows that ssAAV2-smCBA-STC-1-FLAG reduces IOP in a sustained fashion. FIG. 6A is a line plot showing sustained IOP reduction following intracameral injection with ssAAV2-smCBA-STC-1-FLAG. This experiment is an extended timeline of observation from the same initial experiment described in FIG. 3-4. The x-axis represents the longitudinal time length of the experiment in three phases: baseline (pre-injection), weeks post-injection, and re-injection. The number of mice measured in each phase are indicated below. The y-axis represents the value of IOP in units of mmHg. Arrows indicate the point of administration of the first and second injection in the experiment. Following a single intracameral injection with ssAAV2-smCBA-STC-1-FLAG (▪) or PBS (♦) at time 0 weeks after injection, IOP was reduced and remained lower than baseline pressure and fellow control eye for 28 weeks. At 28 weeks, a second intracameral injection of ssAAV-smCBA-STC-1-FLAG was performed. IOP was reduced to levels at or lower than first injection. FIG. 6B is a line graph showing sustained IOP reduction following intracameral injection with ssAAV2-smCBA-STC-1-FLAG. IOP measurements were collected longitudinally for 39 weeks in C57BL/6J with no difference in baseline IOP between fellow eyes receiving single intracameral injection of ssAAV2-smCBA-STC-1-FLAG in one eye (x) and PBS control in the fellow eye (intracameral injection of ssAAV2-smCBA-STC-1-FLAG in one eye (●) and PBS control in the fellow eye (Δ). Two groups of six animals each were injected with a second intracameral injection of ssAAV2-smCBA-STC-1-FLAG in one eye (●) and PBS control in the fellow eye (▪). The x-axis represents the longitudinal time length of the experiment from baseline (pre-injection) to 39 weeks post-first injection. The number of mice measured in each phase are indicated below. The y-axis represents the value of IOP in units of mmHg. Arrows indicate the point of administration of the first and second injection in the experiment. FIG. 6C is a bar graph showing average weekly IOP measurements in ssAAV2-smCBA-STC-1-FLAG injected eyes when compared to the contralateral eye. The x-axis represents average IOP measurements of PBS injected (▪) and ssAAV2-smCBA-STC-1-FLAG injected (□) mice at different months over the course of the experiment. The percent change in IOP and statistical significance (P-value) between conditions are listed below each time point. The y-axis represents the value of IOP in units of mmHg.

FIG. 7A-FIG. 7B shows that ssAAV2-smCBA-STC-1-FLAG reduces IOP compared to ssAAV2-smCBA-GFP. FIG. 7A is a line graph showing average IOP measurements in mice only administered one ssAAV2-smCBA-GFP injection (●), three month-old mice (n=22) only administered one ssAAV2-smCBA-STC-1-FLAG injection (∘), mice administered two injections of ssAAV2-smCBA-GFP (horizontal stripe circle), and mice administered two injections of ssAAV2-smCBA-STC-1-FLAG (diagonal stripe circle). The x-axis represents the longitudinal time length of the experiment from baseline (pre-injection) to 18 weeks post-first injection. The number of mice measured in each phase of the experiment are indicated below. The y-axis represents the value of IOP in units of mmHg. Arrows indicate the point of administration of the first and second injections. FIG. 7B is a bar graph showing average IOP measurements in mice administered one or two injections of ssAAV2-smCBA-GFP or ssAAV2-smCBA-STC-1-FLAG. The x-axis represents the treatment groups including mice only administered one ssAAV2-smCBA-GFP injection (left black bar), mice only administered one ssAAV2-smCBA-STC-1-FLAG injection (left white bar), mice administered two injections of ssAAV2-smCBA-GFP (right black bar), and mice administered two injections of ssAAV2-smCBA-STC-1-FLAG (right white bar). The percent change in IOP and statistical significance (P-value) between conditions are listed below each time point. The y-axis represents the value of IOP in units of mmHg.

FIG. 8 is an agarose gel showing transgene expression of STC-1-FLAG by PCR. PCR amplification of a 728 base pair product consistent with STC-1 FLAG transcripts was only found in mice (n=6) administered intracameral ssAAV2-smCBA-STC-1-FLAG injection.

FIG. 9 is a collection of micrographs of IF-stained sections showing ssAAV2-smCBA-STC-1-FLAG is expressed in a sustained fashion 8 weeks post-injection. GFP was labeled with a red fluorochrome while the FLAG tag was labeled with a green fluorochrome. GFP and FLAG staining signals reflect robust protein expression when compared to secondary only controls. At 8 weeks post injection, diffuse intraocular STC-1-FLAG expression was observed in the iridocorneal angle including ciliary body (*), the anterior segment including cornea (#), iris (\hat{ }), lens capsule (>), and retina.

FIG. 10A-FIG. 10B shows STC-1-FLAG expression is retained at 18 weeks in mice administered either single or double ssAAV2-smCBA-STC-1-FLAG intracameral injections. GFP was labeled with a red fluorochrome while the FLAG tag was labeled with a green fluorochrome. GFP and FLAG staining signals reflect robust protein expression when compared to secondary only controls. FIG. 10A is a collection of micrographs of IF-stained sections of mice administered single injections of ssAAV2-smCBA-STC-1-FLAG or ssAAV2-smCBA-GFP constructs. Sustained STC-1-FLAG expression was observed in multiple tissues at week 18. GFP staining displayed a similar pattern of expression to that of STC-1-FLAG demonstrating the ubiquitous expression under the CBA promoter of the AAV2 construct. FIG. 10B is a collection of micrographs of IF-stained sections of mice administered two injections of ssAAV2-smCBA-STC-1-FLAG or ssAAV2-smCBA-GFP constructs (week 0 and week 9) at week 18.

FIG. 11 is a line plot showing sustained IOP reduction following subconjunctival injection with ssAAV2-smCBA-STC-1-FLAG. The x-axis represents the longitudinal time length of the experiment comprising baseline (pre-injection) and weeks post-injection. The y-axis represents the value of IOP in units of mmHg. Arrows indicate the point of administration of the first and second injection in the experiment. Following a single subconjunctival injection with ssAAV2-smCBA-STC-1-FLAG (●, n=8) or ssAAV2-smCBA-GFP (▪, n=8) at time 0 weeks after injection, ssAAV2-smCBA-STC-1-FLAG significantly lowered IOP for several weeks. Following week 13, a second intracameral injection of each AAV condition was performed. IOP was reduced to levels obtained after first injection with ssAAV2-smCBA-STC-1-FLAG.

FIG. 12A-FIG. 12D shows that intravitreal ssAAV2-hSyn1-STC-1-FLAG does not provide IOP reduction but is neuroprotective in the DBA/2J mouse model of pigment dispersion glaucoma. FIG. 12A is a line graph showing similar IOP measurements in 2-month-old DBA/2J mice administered intravitreal injections with ssAAV2-hSyn1-STC-1-FLAG (▪) in one eye and ssAAV2-hSyn1-GFP in the fellow eye (●). IOP was measured weekly for 9 months. The x-axis represents the longitudinal time length of the experiment in three phases: baseline (pre-injection) and weeks and months post-injection. The number of mice measured in each phase are indicated below. The y-axis represents the value of IOP in units of mmHg. There was no difference in IOP between eyes that received an intravitreal injection of ssAAV2-hSyn1-STC-1 compared to those that received intravitreal ssAAV2-hSyn1-GFP. FIG. 12B is a micrograph showing immunofluorescence (IF) staining revealing GFP expression (top panel, red fluorochrome) in ssAAV2-hSyn1-GFP injected eyes and STC-1-FLAG expression (lower panel, green fluorochrome) in ssAAV2-hSyn1-STC-1-FLAG injected eyes. DAPI stain (blue) shows retinal cell nuclei. FIG. 12C is a montage of IF micrographs of DAPI stained retinal sections revealing fewer retinal ganglion cells (RGCs, white arrows) in eyes injected with ssAAV2-hSyn1-GFP (top panel) compared to those injected with ssAAV2-hSyn1-STC-1-FLAG (bottom panel). FIG. 12D is a bar graph showing number of RGCs in ssAAV2-hSyn1-GFP and AAV2-hSyn1-STC-1-FLAG injected mice. The x-axis represents the treatment group. The y-axis represents the average RGC counts per retina.

FIG. 13A-FIG. 13B shows that topical STC-1 but not latanoprost reduces inflammation in the mouse model of experimental autoimmune uveitis. Following induction of experimental autoimmune uveitis (EAU), mice were treated daily with topical latanoprost free acid (LFA; 100 μM) or STC-1 (0.5 μg/mL) in one eye and vehicle in the other eye. At day 18, OCT was performed and retinal thickness was measured in a masked manner. At day 21, eyes were enucleated and processed for hematoxylin and eosin (H&E) staining. FIG. 13A is a collection of micrographs of histological sections from each treatment group stained by H&E. There was a trend toward greater vitreous cell in all LFA treated animals versus control (as denoted by arrows). FIG. 13B is a bar graph showing STC-1 eyes exhibit a significant preservation of retinal thickness compared to vehicle control eyes (P=0.02), whereas there was no significant difference in retinal thickness between LFA and the contralateral control eye.

FIG. 14A-FIG. 14C shows that virally-delivered STC-1 reduces IOP independent of the FP receptor. Following 4 consecutive days of baseline IOP measurements, 3-month-old FP receptor knockout mice (n=6) received a single intracameral injection of ssAAV2-smCBA-STC-1-FLAG in one eye and ssAAV2-smCBA-GFP into the fellow eye. FIG. 14A is a line graph showing reduced IOP in FP receptor knockout mice (n=6) with no difference in baseline IOP between ssAAV2-smCBA-STC-1-FLAG (∘) injected eyes and ssAAV2-smCBA-GFP (●) control injected eyes. The x-axis represents measurement time points beginning at week 0 (baseline) to week 14. The y-axis represents the value of IOP in units of mmHg. FIG. 14B is a bar graph showing ssAAV2-smCBA-STC-1-FLAG reduces IOP in FP receptor knockout mice. The x-axis represents different treatment groups in the baseline and week 1 time points. Average IOP measurements are detailed for each treatment group/timepoint below, including the % change between groups at each time point. The y-axis represents the value of IOP in units of mmHg. Following 4 consecutive days of baseline IOP measurements, 3-month-old FP receptor knockout mice (n=7) received a single intracameral injection of 1 μL ssAAV2-smCBA-STC-1-FLAG (3E+12 VG/mL) in one eye and the same volume and copy number of ssAAV2-smCBA-GFP into the fellow eye. FIG. 14C is bar graph showing differences in average IOP in Ptgfr−/− knockout mice receiving single intracameral injection of ssAAV2-smCBA-STC-1-FLAG in one eye (□) and ssAAV2-smCBA-GFP control (▪) in the fellow eye. The x-axis represents collected measurements for the different treatment groups at Weeks 0 (baseline), 1, 4, 8, and 14. Average IOP measurements are detailed for each treatment group/timepoint below, including the % change between groups at each time point. The y-axis represents the value of IOP in units of mmHg. This experiment is an extended timeline of observation from the same initial experiment described in FIG. 14B.

FIG. 15A-FIG. 15I shows retinal fundus photographs of a patient having uveal melanoma treated with plaque radiotherapy with no radiation papillopathy, papillopathy with pallor only, and papillopathy with pallor and neuroretinal rim thinning. A 73-year-old female was treated for choroidal melanoma in the temporal periphery from the equator or ora serrata. No optic neuropathy was present (FIG. 15A) before plaque radiotherapy, (FIG. 15B) 36 months after radiotherapy, or (FIG. 15C) 60 months after radiotherapy. A 65-year-old female was treated for choroidal melanoma involving the macula. No optic neuropathy was present (FIG. 15D) before plaque radiotherapy. (FIG. 15E) Optic disc edema was noted 24 months after treatment, with (FIG. progression to optic disc pallor after 36 months. A 54-year-old male was treated for choroidal melanoma in the nasal mid-periphery from the macula to equator. No optic neuropathy was present (FIG. 15G) before plaque radiotherapy. (FIG. 15H) Optic disc edema and Drance hemorrhage were noted 12 months after treatment, with (FIG. 15I) progression to optic disc pallor with neuroretinal rim thinning after 36 months.

FIG. 16. is a table showing values of risk factors and demographics for Radiation Papillopathy following Plaque Radiotherapy for Uveal Melanoma. Abbreviations: NA=not applicable. Bold values indicate significant P-value. Initial P-value compares all 3 groups. *Second P-value directly compares the latter two groups with optic neuropathy characterized by pallor only or pallor with additional neuroretinal rim thinning.

FIG. 17A-17C. is a table showing values of risk factors and clinical features for Radiation Papillopathy following Plaque Radiotherapy for Uveal Melanoma. Abbreviations: NA=not applicable, OCT=optical coherence tomography. Bold values indicate significant P-value. Initial P-value compares all 3 groups. *Second P-value directly compares the latter two groups with optic neuropathy characterized by pallor only or pallor with additional neuroretinal rim thinning.

FIG. 18. is a table showing values of risk factors and treatment features for Radiation Papillopathy following Plaque Radiotherapy for Uveal Melanoma. Abbreviations: NA=not applicable, Gy=gray, hr=hour, VEGF=vascular endothelial growth factor. Bold values indicate significant P-value. Initial P-value compares all 3 groups. *Second P-value directly compares the latter two groups with optic neuropathy characterized by pallor only or pallor with additional neuroretinal rim thinning.

FIG. 19A-19B. is a table showing values of risk factors and outcomes for Radiation Papillopathy following Plaque Radiotherapy for Uveal Melanoma. Abbreviations: NA=not applicable, CF=count fingers, HM=hand motion, LP=light perception. Bold values indicate significant P-value. Initial P-value compares all 3 groups. *Second P-value directly compares the latter two groups with optic neuropathy characterized by pallor only or pallor with additional neuroretinal rim thinning.

FIG. 20A-20H. is a table showing values of risk factors and optic disc features for Radiation Papillopathy following Plaque Radiotherapy for Uveal Melanoma, comprising before plaque (FIG. 20A), 6 months after plaque (FIG. 20B), 12 months after plaque (FIG. 20C), 24 months after plaque (FIG. 20D), 36 months after plaque (FIG. 20E), 60 months after plaque (FIG. 120 months after plaque (FIG. 20G) and at any time after plaque (FIG. 20H). Abbreviations: NA=not applicable, IOP=intraocular pressure, C/D=cup to disc ratio. Bold values indicate significant P-value. Initial P-value compares all 3 groups. *Second P-value directly compares the latter two groups with optic neuropathy characterized by pallor only or pallor with additional neuroretinal rim thinning. **P-value given for pallor present versus absent.

FIG. 21. is a table showing values of risk factors and predictors of Papillopathy by Logistic Regression Analysis for Radiation Papillopathy following Plaque Radiotherapy for Uveal Melanoma. Abbreviations: IOP=intraocular pressure, Gy=gray, OR=odds ratio, CI=confidence interval. Bold values indicate significant P-value. Critical values were calculated using the Benjamini-Hochberg procedure with a false discovery rate of 0.10. For any type of papillopathy, the model for multivariate analysis included mean radiation dose to optic disc, IOP at presentation, subfoveal subretinal fluid, prescription depth, and radiation dose to foveola. For papillopathy with neuroretinal rim thinning, the model for multivariate analysis included mean radiation dose to optic disc, IOP maximum, and subfoveal subretinal fluid.

FIG. 22. is a table showing treatment group conditions for ocular hypertensive mouse models. Between experimental weeks 1-3, a sustained and elevated IOP response was observed in the dexamethasone injected group. Following this, mice were randomly assigned to two groups: one group treated with topical PBS (Group 1) while the other group of mice were treated with topical STC-1 (Group 2) during experimental weeks 4-6. During experimental weeks 7-9, dexamethasone-injected animals in Group 2 continued to receive topical STC-1 while treatment was the vehicle-injected fellow eye underwent a medication wash-out period. The final week of each treatment phase was selected (i.e. weeks 3, 6, and 9) for analysis.

FIG. 23A-23D shows that topical STC-1 reduces IOP in the dexamethasone-injected ocular hypertension mouse model. FIG. 23A is a line graph showing increased IOP in dexamethasone-treated mice. The x-axis represents the longitudinal time length of the experiment. The y-axis represents the value of IOP in units of mmHg. Injections of PBS to the left eye (▪) and dexamethasone to the right eye (♦) were performed at week one of the experiment. FIG. 10B) is a line graph showing IOP of both normotensive and hypertensive mice administered with STC-1 is decreased. The x-axis represents the longitudinal time length of the experiment. The y-axis represents the value of IOP in units of mmHg. Injections of PBS to the left eye (▪) and dexamethasone to the right eye (♦) were performed at week one of the experiment. STC-1 topical treatments were initiated at week 4 and lasted until the end of week 6 in the right eye, whereas in the left eye STC-1 administration was carried out from week 4 until study termination. FIG. 10C) is a bar plot showing IOP is reduced in vehicle-injected right eyes (black bar) and dexamethasone-injected left eyes (white bar) with STC-1 administration. The x-axis represents the different treatment groups at different time points (Week 0, Week 3, Week 6, Week 9). The y-axis represents the value of IOP in units of mmHg. FIG. 10D) shows representative micrographs of toluidine-blue stained sections of dexamethasone-induced ocular hypertension mice following administration of PBS (left panel) versus STC-1 (right panel). In both treatment groups, normal-appearing open angles (asterisk), iris (arrow), and ciliary body (chevron) were observed.

FIG. 24A-24C shows that topical STC-1 reduces IOP in the DBA/2J mouse model of chronic ocular hypertension and glaucomatous ocular neuropathy (GON). FIG. 24A is a line graph showing reduced intraocular pressure in the DBA/2J mouse topically administered STC-1 (▪) as compared to PBS (♦). The x-axis represents the longitudinal time length of the experiment. The y-axis represents the fold-change in IOP relative to the baseline measurement for each treatment group. FIG. 24B is a bar plot showing significant decrease in intraocular pressure (IOP) in STC-1 treated mice compared to PBS between experimental days 6-8 (***P<0.001). The x-axis represents the PBS and STC-1 treatment groups. The y-axis represents the value of IOP in units of mmHg. FIG. 24C shows representative micrographs of toluidine blue-stained sections of 14 month-old mice following administration of PBS (left panel) in one eye and STC-1 (right panel) in the fellow eye. Angle closure with synechiae formation (asterisk), iris atrophy (arrow), and pigment-laden macrophages (chevron) are shown.

### DETAILED DESCRIPTION

The present invention provides methods and compositions for treating a patient (e.g., a human) having ocular hypertension and/or one or more diseases or disorders associated with elevated IOP (e.g., glaucoma) or which are responsive to a reduction in IOP (e.g., NTG). The present invention also provides methods and compositions for treating or preventing radiation papillopathy. For example, one or more nucleic acids designed to express a STC-1 polypeptide can be delivered to one or both eyes (e.g., to cells within one or both eyes) of a patient (e.g., a human) having ocular hypertension and/or one or more diseases or disorders associated with elevated IOP or which are responsive to a reduction in IOP to treat the patient. In some embodiments, nucleic acid designed to express a STC-1 polypeptide can be delivered to cells within an eye of a patient (e.g., a human) having ocular hypertension and/or one or more diseases or disorders associated with elevated IOP or which are responsive to a reduction in IOP in the form of a viral vector (e.g., an adeno-associated virus (AAV) vector).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Each of the references cited herein are incorporated by reference in its entirety.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences, for example a nucleic acid sequence encoding an STC-1 protein.

As used herein, the terms “subject”, “host”, and “patient” are used interchangeably. As used herein, a subject is a subject, host, and patient is a mammal such as a non-primate (e.g., cows, pigs, horses, cats, dogs, rats etc.) or a primate (e.g., monkey and human), or, in certain embodiments, a human. In particular embodiments, the patient is a human.

As used herein, “operably linked” refers to positioning of a regulatory element in a vector relative to a nucleic acid encoding a polypeptide in such a way as to permit or facilitate expression of the encoded polypeptide.

The term “elevated” as used herein with respect to an IOP refers to any IOP that is greater than an IOP typically observed in the eye of a healthy patient (e.g., a patient that does have ocular hypertension and/or one or more diseases or disorders associated with elevated IOP such as glaucoma). In some embodiments, an elevated IOP can be any IOP that is greater than about 21 millimeters of mercury (mmHg). For example, a human that has ocular hypertension and/or one or more diseases or disorders associated with elevated IOP can have an IOP that is from about 22 mmHg to about 75 mmHg (e.g., from about 22 to about 65, from about 22 to about 55, from about 22 to about 45, from about 22 to about 35, from about 22 to about 25, from about 25 to about 75, from about 30 to about 75, from about 35 to about 75, from about 40 to about 75, from about 45 to about 75, from about 50 to about 75, from about 55 to about 75, from about 60 to about 75, from about 25 to about 60, from about 30 to about 50, from about 25 to about 35, from about 30 to about from about 35 to about 45, from about 40 to about 50, from about 45 to about 55, from about to about 60, or from about 55 to about 65 mmHg. Methods of measuring IOP are well known in the art, and include, for example, the use of a tonometry, for example, Goldmann Applanation Tonometry (GAT), Perkins tonometry, non-contact tonometry, Tono-pen, Rebound tonometry, pneumotonometry, ICare, and other applicable methods. In some embodiments, a suitable technique for determining IOP can be performed using GAT.

Methods of Reducing IOP

Any appropriate method can be used to deliver one or more nucleic acids designed to express a STC-1 polypeptide to one or both eyes (e.g., to cells within one or both eyes) of a patient for the purposes of reducing IOP in a patient in need thereof. In some embodiments, nucleic acid encoding a STC-1 polypeptide can be administered to one or both eyes (e.g., to cells within one or both eyes) of a patient by direct injection of naked nucleic acid molecules (e.g., such that STC-1 polypeptides are expressed within one or both eyes). In some embodiments, nucleic acid encoding a STC-1 polypeptide can be administered to one or both eyes (e.g., to cells within one or both eyes) of a patient using one or more vectors (e.g., such that STC-1 polypeptides are expressed within one or both eyes). In some embodiments, nucleic acid encoding a STC-1 polypeptide can be administered to one or both eyes (e.g., to cells within one or both eyes) of a patient using one or more carrier molecules (e.g., peptide carriers and nanoparticles such as liposomes). In some embodiments, nucleic acid encoding a STC-1 polypeptide can be administered to one or both eyes (e.g., to cells within one or both eyes) of a patient using one or more targeting molecules.

In some embodiments, a patient (e.g., a human) can be administered a single administration (e.g., a single injection) of one or more nucleic acids designed to express a STC-1 polypeptide to one or both eyes (e.g., to cells within one or both eyes) of the patient.

In some embodiments, a patient (e.g., a human) can be administered two or more (e.g., two, three, four, or more) administrations (e.g., injections) of one or more nucleic acids designed to express a STC-1 polypeptide to one or both eyes (e.g., to cells within one or both eyes) of the patient. In some embodiments, when a patient is administered two or more administrations of one or more nucleic acids designed to express a STC-1 polypeptide to one or both eyes of the patient the administrations are about once a week. In some embodiments, when a patient is administered two or more administrations of one or more nucleic acids designed to express a STC-1 polypeptide to one or both eyes of the patient the administrations are no more frequent than about 2 weeks. In some embodiments, two or more administrations of one or more nucleic acids designed to express a STC-1 polypeptide to one or both eyes of a patient can be administered about a week apart, at least 2 weeks apart, at least 30 days apart, at least 180 days apart, at least 1 year apart, at least 3 years apart, at least 5 years apart, at least 8 years apart, or at least 10 years apart. For example, two or more administrations of one or more nucleic acids designed to express a STC-1 polypeptide to one or both eyes of a patient can be administered with at least 1 week, 2 weeks, at least 30 days, at least 180 days, at least 1 year, at least 3 years, at least 5 years, at least 8 years, or at least 10 years apart separating the administrations. In some embodiments, two or more administrations of one or more nucleic acids designed to express a STC-1 polypeptide to one or both eyes of a patient can be administered from about 2 weeks to 10 years apart (e.g., from about 2 weeks to about 9 years, from about 2 weeks to about 8 years, from about 2 weeks to about 7 years, from about 2 weeks to about 6 years, from about 2 weeks to about 5 years, from about 2 weeks to about 4 years, from about 2 weeks to about 3 years, from about 2 weeks to about 2 years, from about 2 weeks to about 1 year, from about 2 weeks to about 6 months, from about 2 weeks to about 180 days, or from about 2 weeks to about 1 month, from about 30 days to about 10 years, from about 30 days to about 9 years, from about 30 days to about 8 years, from about 30 days to about 7 years, from about 30 days to about 6 years, from about 30 days to about 5 years, from about 30 days to about 4 years, from about 30 days to about 3 years, from about 30 days to about 2 years, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 year to about 3 years, from about 2 years to about 4 years, from about 3 years to about 5 years, from about 4 years to about 6 years, from about 5 years to about 7 years, from about 6 years to about 8 years, or from about 7 years to about 9 years apart) separating the administrations. For example, two or more administrations of one or more nucleic acids designed to express a STC-1 polypeptide to one or both eyes of a patient can be administered from about 2 weeks to 10 years apart (e.g., from about 2 weeks to about 9 years, from about 2 weeks to about 8 years, from about 2 weeks to about 7 years, from about 2 weeks to about 6 years, from about 2 weeks to about 5 years, from about 2 weeks to about 4 years, from about 2 weeks to about 3 years, from about 2 weeks to about 2 years, from about 2 weeks to about 1 year, from about 2 weeks to about 6 months, from about 2 weeks to about 180 days, or from about 2 weeks to about 1 month, from about 30 days to about 10 years, from about 30 days to about 9 years, from about 30 days to about 8 years, from about 30 days to about 7 years, from about 30 days to about 6 years, from about 30 days to about 5 years, from about 30 days to about 4 years, from about 30 days to about 3 years, from about 30 days to about 2 years, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 year to about 3 years, from about 2 years to about 4 years, from about 3 years to about 5 years, from about 4 years to about 6 years, from about 5 years to about 7 years, from about 6 years to about 8 years, or from about 7 years to about 9 years apart) separating the administrations. In some embodiments, two or more administrations of one or more nucleic acids designed to express a STC-1 polypeptide to one or both eyes of a patient can be administered once a week. In some embodiments, two or more administrations of one or more nucleic acids designed to express a STC-1 polypeptide to one or both eyes of a patient can be administered no more frequently than about 2 weeks. In some embodiments, two or more administrations of one or more nucleic acids designed to express a STC-1 polypeptide to one or both eyes of a patient can be administered no more frequently than about 30 days. In some embodiments, two or more administrations of one or more nucleic acids designed to express a STC-1 polypeptide to one or both eyes of a patient can be administered no more frequently than about 90 days. In some embodiments, two or more administrations of one or more nucleic acids designed to express a STC-1 polypeptide to one or both eyes of a patient can be administered no more frequently than about 120 days. In some embodiments, two or more administrations of one or more nucleic acids designed to express a STC-1 polypeptide to one or both eyes of a patient can be administered no more frequently than about 180 days.

In general, one aspect of this present invention features methods for treating patients, for example a human, having ocular hypertension or disorders responsive to a reduction in IOP. The methods can include, or consist essentially of, administering a nucleic acid encoding a STC-1 polypeptide or a variant of the STC-1 polypeptide to cells within an eye of the patient, where the STC-1 polypeptide or the variant is expressed by the cells, and where an IOP of the eye is reduced. The patient can have a disease or disorder associated with elevated IOP. The patient can have a disease or disorder that is responsive to a lowering of IOP or whose symptoms or etiology is ameliorated with a reduction in IOP. The patient can be a human. The nucleic acid can encode the STC-1 polypeptide. The nucleic acid can encode the variant of the STC-1 polypeptide. The nucleic acid can be administered to the cells in the form of a viral vector. The viral vector can be an AAV vector, for example, but not limited to AAV2, AAV8, and AAV9. In some embodiments, the AAV vector is an AAV2 vector. In an alternative embodiment, the nucleic acid is administered to the cells in the form of a non-viral vector. The non-viral vector can be an expression plasmid. In some embodiments, the nucleic acid can be administered to the cells using a carrier molecule. The carrier molecule can be a nanoparticle. The nucleic acid can be operably linked to a promoter sequence. The promoter sequence can be a ubiquitous promoter (e.g., a chicken β-actin (CBA) promoter). The promoter can be a cell-specific promoter (e.g., a synapsin 1 (SYN1) promoter. The administration can be an intracameral injection. The administration can be a subconjunctival injection. The method can be effective to reduce the IOP by about 10% to about 50% or greater, for example 10%, 15%, 20%, 25%, 30%, 40%, 45%, 50%, 55%, 60%, or greater than 60%, from a baseline measurement taken before treatment. The method can be effective to reduce the IOP to between 10 mmHg to about 21 mmHg. The method can be effective to reduce the IOP by about 1 mm Hg, 2 mmHg, 3 mmHg, 4 mmHg, 5 mmHg, 6 mmHg, 7 mmHg, 8 mmHg, 9 mmHg, 10 mmHg, or greater than 10 mmHg from a baseline measurement taken before treatment. The method can be effective to reduce the IOP for from about 1 day to about 2 years. The method can include a single administration of the nucleic acid encoding the STC-1 polypeptide or the variant of the STC-1 polypeptide. The single administration can be effective for the cells to express the STC-1 polypeptide or the variant of the STC-1 polypeptide for from about 2 weeks to about 10 years. The method can include two or more administrations, for example 2, 3, 4, 5, 6, 7, 8, 9, 10, 11, 12, 13, 14, 15 or more than 15 administrations of the nucleic acid encoding the STC-1 polypeptide or the variant of the STC-1 polypeptide. The administrations can be administered from about 1 weeks to about 10 years apart. The method can include three administrations of the nucleic acid encoding the STC-1 polypeptide or the variant of the STC-1 polypeptide.

In another aspect, provided herein are methods for treating a patient having a disease or disorder associated with elevated IOP. The methods can include, or consist essentially of, administering nucleic acid encoding a STC-1 polypeptide or a variant of the STC-1 polypeptide to cells within an eye of the patient, where the STC-1 polypeptide or the variant is expressed by the cells, and where an IOP of the eye is reduced. The disease or disorder associated with elevated IOP can be glaucoma or radiation papillopathy. The patient can be a human. The nucleic acid can encode the STC-1 polypeptide. The nucleic acid can encode the variant of the STC-1 polypeptide. The nucleic acid can be administered to the cells in the form of a viral vector. The viral vector can be an AAV vector. The AAV vector can be an AAV2 vector. The nucleic acid can be administered to the cells in the form of a non-viral vector. The non-viral vector can be an expression plasmid. The nucleic acid can be administered to the cells using a carrier molecule. The carrier molecule can be a nanoparticle. The nucleic acid can be operably linked to a promoter sequence. The promoter sequence can be a ubiquitous promoter (e.g., a CBA promoter). The promoter can be a cell-specific promoter (e.g., a SYN1 promoter). The administration can be an intracameral injection. The administration can be an intracameral injection. The administration can be a subconjunctival injection. The method can be effective to reduce the IOP by about 10% to about 50% or greater, for example 10%, 15%, 20%, 25%, 30%, 40%, 45%, 50%, 55%, 60%, or greater than 60%, from a baseline measurement taken before treatment. The method can be effective to reduce the IOP to between 10 mmHg to about 21 mmHg. The method can be effective to reduce the IOP by about 1 mm Hg, 2 mmHg, 3 mmHg, 4 mmHg, 5 mmHg, 6 mmHg, 7 mmHg, 8 mmHg, 9 mmHg, 10 mmHg, or greater than 10 mmHg from a baseline measurement taken before treatment. The method can be effective to reduce the IOP for from about 1 day to about 2 years. The method can include a single administration of the nucleic acid encoding the STC-1 polypeptide or the variant of the STC-1 polypeptide. The single administration can be effective for the cells to express the STC-1 polypeptide or the variant of the STC-1 polypeptide for from about 2 weeks to about 10 years. The method can include two or more administrations, for example 2, 3, 4, 5, 6, 7, 8, 9, 10, 11, 12, 13, 14, 15 or more than 15 administrations of the nucleic acid encoding the STC-1 polypeptide or the variant of the STC-1 polypeptide. The administrations can be administered from about 1 weeks to about 10 years apart. The method can include three administrations of the nucleic acid encoding the STC-1 polypeptide or the variant of the STC-1 polypeptide.

In another aspect, provided herein are methods for treating a patient having glaucomatous optic neuropathy. The methods can include, or consist essentially of, administering nucleic acid encoding a STC-1 polypeptide or a variant of the STC-1 polypeptide to cells within an eye of the patient, where the STC-1 polypeptide or the variant is expressed by the cells, and where an IOP of the eye is reduced. The patient can be a human. The nucleic acid can encode the STC-1 polypeptide. The nucleic acid can encode the variant of the STC-1 polypeptide. The nucleic acid can be administered to the cells in the form of a viral vector. The viral vector can be an AAV vector. The AAV vector can be an AAV2 vector. The nucleic acid can be administered to the cells in the form of a non-viral vector. The non-viral vector can be an expression plasmid. The nucleic acid can be administered to the cells using a carrier molecule. The carrier molecule can be a nanoparticle. The nucleic acid can be operably linked to a promoter sequence. The promoter sequence can be a ubiquitous promoter (e.g., a CBA promoter). The promoter can be a cell-specific promoter (e.g., a SYN1 promoter). The administration can be an intracameral injection. The administration can be a subconjunctival injection. The method can be effective to reduce the IOP by about 10% to about 50% or greater, for example 10%, 15%, 20%, 25%, 30%, 40%, 45%, 50%, 55%, 60%, or greater than 60%, from a baseline measurement taken before treatment. The method can be effective to reduce the IOP to between 10 mmHg to about 21 mmHg. The method can be effective to reduce the IOP by about 1 mm Hg, 2 mmHg, 3 mmHg, 4 mmHg, 5 mmHg, 6 mmHg, 7 mmHg, 8 mmHg, 9 mmHg, 10 mmHg, or greater than 10 mmHg from a baseline measurement taken before treatment. The method can be effective to reduce the IOP for from about 1 day to about 2 years. The method can include a single administration of the nucleic acid encoding the STC-1 polypeptide or the variant of the STC-1 polypeptide. The single administration can be effective for the cells to express the STC-1 polypeptide or the variant of the STC-1 polypeptide for from about 2 weeks to about 10 years. The method can include two or more administrations, for example 2, 3, 4, 5, 6, 7, 8, 9, 10, 11, 12, 13, 14, 15 or more than 15 administrations of the nucleic acid encoding the STC-1 polypeptide or the variant of the STC-1 polypeptide. The administrations can be administered from about 1 weeks to about 10 years apart. The method can include three administrations of the nucleic acid encoding the STC-1 polypeptide or the variant of the STC-1 polypeptide.

In another aspect, provided herein are methods for reducing ocular inflammation in a patient. The methods can include, or consist essentially of, administering nucleic acid encoding a STC-1 polypeptide or a variant of said STC-1 polypeptide to cells within an eye of a patient, where the STC-1 polypeptide or the variant is expressed by the cells. The patient can be a human. The nucleic acid can encode the STC-1 polypeptide. The nucleic acid can encode the variant of the STC-1 polypeptide. The nucleic acid can be administered to the cells in the form of a viral vector. The viral vector can be an AAV vector. The AAV vector can be an AAV2 vector. The nucleic acid can be administered to the cells in the form of a non-viral vector. The non-viral vector can be an expression plasmid. The nucleic acid can be administered to the cells using a carrier molecule. The carrier molecule can be a nanoparticle. The nucleic acid can be operably linked to a promoter sequence. The promoter sequence can be a ubiquitous promoter (e.g., a CBA promoter). The promoter can be a cell-specific promoter (e.g., a SYN1 promoter). The administration can be an intracameral injection. The administration can be an intravitreal injection. The administration can be a subconjunctival injection.

In another aspect, provided herein are methods for providing neuroprotection of an ocular neuron in a patient. The methods can include, or consist essentially of, administering nucleic acid encoding a STC-1 polypeptide or a variant of said STC-1 polypeptide to cells within an eye of a patient, where the STC-1 polypeptide or the variant is expressed by the cells. The patient can be a human. The nucleic acid can encode the STC-1 polypeptide. The nucleic acid can encode the variant of the STC-1 polypeptide. The nucleic acid can be administered to the cells in the form of a viral vector. The viral vector can be an AAV vector. The AAV vector can be an AAV2 vector. The nucleic acid can be administered to the cells in the form of a non-viral vector. The non-viral vector can be an expression plasmid. The nucleic acid can be administered to the cells using a carrier molecule. The carrier molecule can be a nanoparticle. The nucleic acid can be operably linked to a promoter sequence. The promoter sequence can be a ubiquitous promoter (e.g., a CBA promoter). The promoter can be a cell-specific promoter (e.g., a SYN1 promoter). The administration can be an intracameral injection. The administration can be an intravitreal injection. The administration can be a subconjunctival injection.

In some embodiments, the methods and materials described herein can be used to treat neuropathy associated with ocular hypertension and/or one or more diseases or disorders associated with elevated IOP (e.g., can be used to treat GON). For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP such as glaucoma) to slow, delay, or prevent progression of neuropathy associated with ocular hypertension and/or one or more diseases or disorders associated with elevated IOP (e.g., to slow, delay, or prevent the development of GON).

In some cases, the methods and materials described herein can be used to treat ocular hypertension. For example, one or more nucleic acids designed to express a STC-1 polypeptide can be delivered to one or both eyes (e.g., to cells within one or both eyes) of a patient (e.g., a human) in need thereof (e.g., a human having ocular hypertension) to slow, delay, or prevent progression of ocular hypertension (e.g., to slow, delay, or prevent the development of ocular hypertension).

In some embodiments, the methods and materials described herein can be used to reduce or eliminate one or more symptoms of ocular hypertension and/or one or more diseases or disorders associated with elevated IOP (e.g., glaucoma). For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP) to reduce or eliminate one or more symptoms of ocular hypertension and/or one or more diseases or disorders associated with elevated IOP. Examples of symptoms of ocular hypertension and one or more diseases or disorders associated with elevated IOP such as glaucoma include, without limitation, blind spots in peripheral and/or central vision in one or both eyes, tunnel vision (e.g., in advanced stage glaucoma), headaches, pain (e.g., eye pain), nausea, vomiting, blurred vision, halos around lights, and eye redness. In some embodiments, the materials and methods described herein can be used to reduce the severity of one or more symptoms of ocular hypertension and/or one or more diseases or disorders associated with elevated IOP (e.g., glaucoma) in a patient (e.g., a human) by, for example, 10, 20, 30, 40, 50, 60, 70, 80, 90, 95, or more percent. In embodiments where a glaucoma is an asymptomatic glaucoma, the methods and materials described herein can be used to delay or prevent the development of one or more symptoms of glaucoma. For example, one or more nucleic acids designed to express a STC-1 polypeptide can be delivered to one or both eyes (e.g., to cells within one or both eyes) of a patient (e.g., a human) in need thereof (e.g., a human having glaucoma) to delay or prevent the development of one or more symptoms of glaucoma.

In some embodiments, the methods and materials described herein can be used to reduce IOP. For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP) to reduce the IOP in one or both eyes of the patient, or a disorder that is responsive to the reduction of IOP (e.g., NTG). In some embodiments, the methods and materials described herein can be used to achieve an IOP that is effective to reduce the risk of a patient developing progressive nerve damage. In some embodiments, the methods and materials described herein can be used to achieve an IOP of from about 21 mmHg to about 10 mmHg (e.g., from about 20 mmHg to about 10 mmHg, from about 18 mmHg to about 10 mmHg, from about 15 mmHg to about 10 mmHg, from about 12 mmHg to about 10 mmHg, from about 21 mmHg to about 13 mmHg, from about 21 mmHg to about 15 mmHg, from about 21 mmHg to about 17 mmHg, from about 21 mmHg to about 19 mmHg, from about 20 mmHg to about 12 mmHg, from about 18 mmHg to about 14 mmHg, from about 15 mmHg to about 12 mmHg, from about 18 mmHg to about 15 mmHg, or from about 20 mmHg to about 18 mmHg). In some embodiments, the methods and materials described herein can be used to reduce the IOP in one or both eyes of a patient (e.g., a human) by, for example, 10, 20, 30, 40, 50, 60, 70, 80, 90, 95, or more percent. In some embodiments, one or more nucleic acids designed to express a STC-1 polypeptide can be delivered to one or both eyes (e.g., to cells within one or both eyes) of a patient (e.g., a human) to reduce the IOP in an eye of the patient by at least about 10%. For example, one or more nucleic acids designed to express a STC-1 polypeptide can be delivered to one or both eyes (e.g., to cells within one or both eyes) of a patient (e.g., a human) to reduce the IOP in an eye of the patient by from about 10% to about 40% (e.g., from about 10% to about 40%, from about 15% to about 40%, from about 20% to about 40%, from about 25% to about 40%, from about 30% to about 40%, from about 35% to about 40%, from about 10% to about 35%, from about 15% to about 35%, from about 10% to about 30%, from about 15% to about 30%, from about 20% to about 30%, from about 10% to about 25%, from about 15% to about 25%, from about 10% to about 20%, from about 20% to about 30%, or from about 25% to about 35%).

In embodiments where the methods and materials described herein are used to reduce IOP, the reduced IOP can be a sustained reduction. For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP) to reduce the IOP in one or both eyes of the patient for from about from about 1 day to about 2 years (e.g., from about 1 day to about 1.5 years, from about 1 day to about 1 year, from about 1 day to about 9 months, from about 1 day to about 6 months, from about 1 day to about 3 months, from about 1 day to about 1 month, from about 1 day to about 2 weeks, from about 2 weeks to about 2 years, from about 1 month to about 2 years, from about 3 months to about 2 years, from about 6 months to about 2 years, from about 1 year to about 2 years, from about 1 week to about 1 year, from about 2 weeks to about 9 months, from about 1 month to about 6 months, from about 1 week to about 1 month, from about 1 month to about 3 months, from about 3 months to about 6 months, from about 6 months to about 9 months, from about 9 months to about 1 year, or from about 1 year to about 1.5 years).

In some embodiments, the methods and materials described herein can be used to reduce or eliminate uveitis (ocular inflammation) in one or both eyes of a patient (e.g., a human). For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP) to reduce or eliminate inflammation in one or both eyes. In some embodiments, the materials and methods described herein can be used to reduce inflammation in one or both eyes of a patient (e.g., a human) by, for example, 10, 20, 30, 40, 50, 60, 70, 80, 90, 95, or more percent.

In some embodiments, the methods and materials described herein can be used for neuroprotection of one or more neurons within a patient (e.g., a human). For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP such as glaucoma) to slow, delay, or prevent damage, degeneration, and/or impairment of function of one or more neurons within one or both eyes of a patient (e.g., a human). For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP) to slow, delay, or prevent reduction of retinal thickness within one or both eyes of a patient (e.g., a human). For example, one or more nucleic acids designed to express a STC-1 polypeptide can be delivered to one or both eyes (e.g., to cells within one or both eyes) of a patient (e.g., a human) in need thereof (e.g., a human having ocular hypertension and/or one or more diseases or disorders associated with elevated IOP) to preserve retinal thickness within one or both eyes of a patient (e.g., a human). In some embodiments, the expression of a STC-1 polypeptide can result in neuroprotection that is independent of IOP reduction.

In some embodiments, when a patient (e.g., a human) having ocular hypertension and/or one or more diseases or disorders associated with elevated IOP (e.g., glaucoma) or a disease or disorder responsive to a reduction in IOP is administered one or more nucleic acids designed to express a STC-1 polypeptide, the one or more nucleic acids designed to express a STC-1 polypeptide can be well tolerated by the patient. For example, when one or more nucleic acids designed to express a STC-1 polypeptide are delivered to one or both eyes (e.g., to cells within one or both eyes) of a patient (e.g., a human) in need thereof (e.g., a human having ocular hypertension and/or one or more diseases or disorders associated with elevated IOP), the patient can experience minimal, reduced, or no side effects (e.g., conjunctival hyperemia, surface irritation, pigmentation of the iris and periocular skin, orbital fat atrophy, hypertrichosis, intraocular inflammation, reactivation of herpes simplex keratitis, and macular edema).

Any appropriate patient can be treated as described herein (e.g., by delivering one or more nucleic acids designed to express a STC-1 polypeptide to one or both eyes of the patient). In some embodiments, a patient can have ocular hypertension and/or one or more diseases or disorders associated with elevated IOP. In some embodiments, a patient can have a disease or disorder that is responsive to a reduction in IOP, for example NTG. In some embodiments, a patient can be non-responsive to PGF2α analogues (e.g., latanoprost). In some embodiments, a patient can be non-responsive to beta blockers. In some embodiments, a patient can be non-responsive to carbonic anhydrase inhibitors. In some embodiments, a patient can be non-responsive to Rho kinase inhibitors. In some embodiments, a patient can be a human that is about 60 years of age or older. In some embodiments, a patient can be using or can have a history of using steroid medication (e.g., corticosteroid eye drops). In some embodiments, a patient can have one or more medical conditions (e.g., diabetes, heart disease, high blood pressure, and/or sickle cell anemia). In some embodiments, a patient can have one or more corneas that are thin in the center. In some embodiments, a patient can have one or more corneas that are thick in the center. In some embodiments, a patient can have had an eye injury. In some embodiments, a patient can have had an eye surgery. Examples of patients that can be treated as described herein include, without limitation, humans, non-human primates such as monkeys, dogs, cats, horses, cows, pigs, sheep, llamas, mice, rats, guinea pigs, and rabbits.

When treating a patient (e.g., a human) having ocular hypertension and/or one or more diseases or disorders associated with elevated IOP (e.g., glaucoma) as described herein (e.g., by delivering one or more nucleic acids designed to express a STC-1 polypeptide to one or both eyes of the patient), the disease or disorder associated with elevated IOP can be any type of disease or disorder associated with elevated IOP. An example of a disease or disorder associated with elevated IOP includes, without limitation, glaucoma. When treating a patient (e.g., a human) having glaucoma as described herein, the glaucoma can be any type of glaucoma. In some embodiments, a glaucoma can include GON. In some embodiments, a glaucoma can be a high pressure glaucoma. Examples of types of glaucoma include, without limitation, primary open angle glaucoma (POAG; also known as chronic open angle glaucoma, chronic simple glaucoma and glaucoma simplex), primary angle closure glaucoma, pediatric glaucoma, pseudo-exfoliative glaucoma, pigmentary glaucoma, traumatic glaucoma, neovascular glaucoma, and irido corneal endothelial glaucoma, and closed-angle glaucoma (e.g., narrow angle glaucoma and acute congestive glaucoma).

When treating a patient (e.g., a human) having a disease or disorder responsive to a reduction or lowering of IOP in an affected eye as described herein (e.g., by delivering one or more nucleic acids designed to express a STC-1 polypeptide to one or both eyes of the patient), the disease or disorder responsive to a reduction or lowering of IOP can be any disease or disorder responsive to a reduction or lowering of IOP. An example of such a disease or disorder responsive to a reduction in or lowering of IOP is normal-tension glaucoma (NTG). In some embodiments, the patient has NTG, and does not have a history of elevated IOP, for example, a history with chronic or acute IOP above 22 mmHg.

In some embodiments, a patient (e.g., a human) can be identified as having ocular hypertension and/or one or more diseases or disorders associated with elevated IOP (e.g., glaucoma), or responsive to the reduction in or lowering of IOP using any appropriate diagnostic technique. For example, dilated eye examination (e.g., to look for optic nerve damage), ocular tonometry (to measure IOP), visual field test, pachymetry (e.g., to measure corneal thickness), gonioscopy (e.g., to measure the drainage angle), optical coherence tomography, and/or electroretinogram analysis can be used to diagnose a human as having ocular hypertension and/or one or more diseases or disorders associated with elevated IOP.

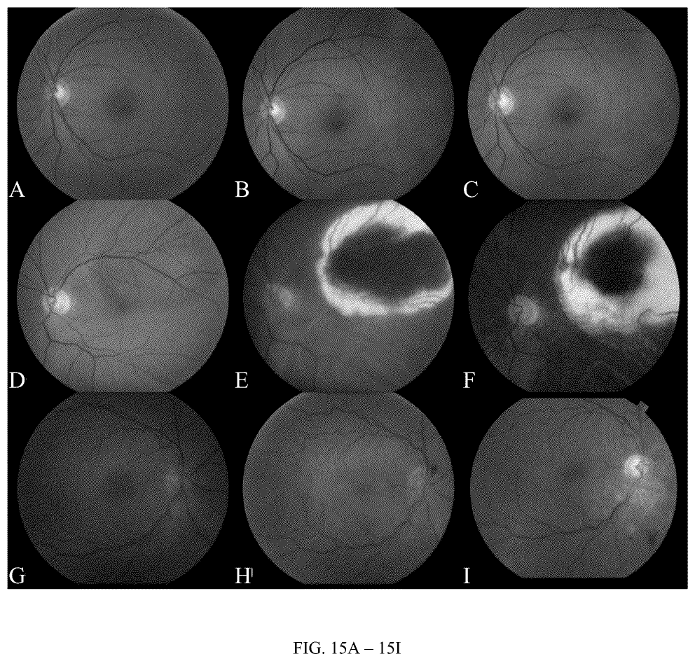

In an alternative aspect, the methods and compositions described herein are used to prevent or reduce the development of radiation papillopathy, which is a complication of irradiation of intraocular tumors, for example in the parapapillary area. As described in Example 5 and FIGS. 15-21 below, it has been discovered that individuals who experience increased IOP before or during radiation treatments for an ocular tumor are at a significant risk for developing radiation papillopathy. Accordingly, in some embodiments, the compositions and methods described herein are used to treat or prevent radiation papillopathy in a patient, for example a human, receiving radiation for the treatment of an ocular tumor. In some embodiments, the patient is administered a composition described herein prior to initiation of the radiation. In some embodiments, the patient is administered a composition described herein two or more times during the course of radiation treatment. In some embodiments, the patient has an elevated IOP prior to administration of the radiation, for example, an IOP greater than 22 mmHg. In some embodiments, the patient has an IOP greater than 18 mmHg prior to receiving radiation therapy. In some embodiments, the patient does not have a history of elevated IOP prior to receiving radiation therapy. In some embodiments, the intraocular tumor is, for example but not limited to, a melanoma, for example a choroidal melanoma or uveal melanoma, a choroidal hemangioma, a choroidal nevus, an iris tumor, a retinoblastoma, a lacrimal gland tumor, or an intraocular lymphoma. In some embodiments, the compositions described herein are administered to the patient within 1 month, 2 weeks, one week, 72 hours, 48 hours, 36 hours, 24 hours, 12, hours, 8 hours, 6 hours, 4 hours, or less prior to receiving the radiation treatment. In some embodiments, the rAAV administration to the eye comprises administration within 4 hours of the administration of radiation dose to eye. In some embodiments, the patient is administered a composition described herein to prevent radiation papillopathy after receiving radiation treatment to the eye, for example, immediately after receiving radiation, 1 day, 3 days, 7 days, 14 days, 30 days, 45 days, 60 days, 90 days, 180 days, 1 year, or greater after receiving radiation treatment to the eye.

Nucleic Acid Sequences Encoding Stanniocalcin-1

Nucleic acids designed to express any appropriate STC-1 polypeptide can be delivered to one or both eyes (e.g., to cells within one or both eyes) of a patient (e.g., a human) as described herein. Examples of STC-1 polypeptides and nucleic acids encoding STC-I polypeptides include, without limitation, those set forth in Table 1. In some embodiments, a nucleic acid encoding a STC-1 polypeptide can contain a nucleic acid sequence encoding a detectable label. For example, a vector can include a nucleic acid encoding a STC-1 polypeptide and nucleic acid encoding a detectable label positioned such that the encoded polypeptide is a fusion polypeptide that includes a STC-1 polypeptide fused to a detectable polypeptide. In some embodiments, a detectable label can be a peptide tag. Examples of detectable labels that can be used as described herein include, without limitation, an HA tag, a Myc-tag, a FLAG-tag, and a fluorescent polypeptide (e.g., a green fluorescent polypeptide (GFP)).

In some embodiments, the STC-1 nucleic acid is derived from the wild-type, consensus, or native human STC-1 mRNA sequence. In some embodiments, the nucleic acid is derived from the wild-type or native human STC-1 coding sequence (SEQ ID NO:1)(NCBI CCDS 6043.1), or a nucleic acid sequence at least 70%, at least 75%, at least 80%, at least 90%, at least 95%, at least 96%, at least 97%, at least 98%, or at least 99% identical to SEQ ID NO:1. In some embodiments, the nucleic acid is SEQ ID NO:1. In some embodiments, the STC-1 has been codon optimized for expression from an AAV vector. In some embodiments, the STC-1 has been codon optimized for expression in a human ocular cell. In some embodiments, the nucleic acid of SEQ ID NO:1 further comprising a nucleic acid sequence that encodes a polypeptide tag.

In some embodiments, the nucleic acid encodes an STC-1 polypeptide having an amino acid sequence of SEQ ID NO:2, or an amino acid sequence at least 70%, at least 75%, at least 80%, at least 90%, at least 95%, at least 96%, at least 97%, at least 98%, or at least 99% identical to SEQ ID NO:2. In some embodiments, the nucleic acid encodes an STC-1 polypeptide having an amino acid sequence of SEQ ID NO:2. In some embodiments, the amino acid of SEQ ID NO:2 further comprises an amino acid sequence tag. In some embodiments, the amino acid sequence tag is selected from DYKDDDDK (SEQ ID NO:30), SDYKDDDDK (SEQ ID NO:31), or ASDYKDDDDK (SEQ ID NO:32).

In some embodiments, the nucleic acid is derived from the wild-type or native human STC-1 propeptide coding sequence (SEQ ID NO:3), or a nucleic acid sequence at least 70%, at least 75%, at least 80%, at least 90%, at least 95%, at least 96%, at least 97%, at least 98%, or at least 99% identical to SEQ ID NO:3. In some embodiments, the nucleic acid is SEQ ID NO:3. In some embodiments, the STC-1 has been codon optimized for expression from an AAV vector. In some embodiments, the STC-1 has been codon optimized for expression in a human ocular cell. In some embodiments, the nucleic acid of SEQ ID NO:3 further comprising a nucleic acid sequence that encodes a polypeptide tag.

In some embodiments, the nucleic acid encodes an STC-propeptide having an amino acid sequence of SEQ ID NO:4, or an amino acid sequence at least 70%, at least 75%, at least 80%, at least 90%, at least 95%, at least 96%, at least 97%, at least 98%, or at least 99% identical to SEQ ID NO:4. In some embodiments, the nucleic acid encodes an STC-1 propeptide having an amino acid sequence of SEQ ID NO:4. In some embodiments, the amino acid of SEQ ID NO:4 further comprises an amino acid sequence tag. In some embodiments, the amino acid sequence tag is selected from DYKDDDDK (SEQ ID NO:30), SDYKDDDDK (SEQ ID NO:31), or ASDYKDDDDK (SEQ ID NO:32).

In some embodiments, the nucleic acid is derived from the wild-type or native human STC-1 chain polypeptide coding sequence SEQ ID NO:5, or a nucleic acid sequence at least 70%, at least 75%, at least 80%, at least 90%, at least 95%, at least 96%, at least 97%, at least 98%, or at least 99% identical to SEQ ID NO:5. In some embodiments, the nucleic acid is SEQ ID NO:5. In some embodiments, the STC-1 has been codon optimized for expression from an AAV vector. In some embodiments, the STC-1 has been codon optimized for expression in a human ocular cell. In some embodiments, the nucleic acid of SEQ ID NO:5 further comprising a nucleic acid sequence that encodes a polypeptide tag.

In some embodiments, the nucleic acid encodes an STC-chain polypeptide having an amino acid sequence of SEQ ID NO:6, or an amino acid sequence at least 70%, at least 75%, at least 80%, at least 90%, at least 95%, at least 96%, at least 97%, at least 98%, or at least 99% identical to SEQ ID NO:6. In some embodiments, the nucleic acid encodes an STC-1 chain polypeptide having an amino acid sequence of SEQ ID NO:6. In some embodiments, the amino acid of SEQ ID NO:6 further comprises an amino acid sequence tag. In some embodiments, the amino acid sequence tag is selected from DYKDDDDK (SEQ ID NO:30), SDYKDDDDK (SEQ ID NO:31), or ASDYKDDDDK (SEQ ID NO:32).

In some embodiments, the nucleic acid encoding STC-1 is derived from SEQ ID NO:7, or a nucleic acid sequence at least 70%, at least 75%, at least 80%, at least 90%, at least 95%, at least 96%, at least 97%, at least 98%, or at least 99% identical to SEQ ID NO:7. In some embodiments, the nucleic acid is SEQ ID NO:7. In some embodiments, the STC-1 has been codon optimized for expression from an AAV vector. In some embodiments, the STC-1 has been codon optimized for expression in a human ocular cell.

In some embodiments, the nucleic acid encodes an STC-1 polypeptide having an amino acid sequence of SEQ ID NO:8, or an amino acid sequence at least 70%, at least 75%, at least 80%, at least 90%, at least 95%, at least 96%, at least 97%, at least 98%, or at least 99% identical to SEQ ID NO:8. In some embodiments, the nucleic acid encodes an STC-1 polypeptide having an amino acid sequence of SEQ ID NO:8.

In some embodiments, the nucleic acid is derived from the nucleic acid sequence of SEQ ID NO:9, or a nucleic acid sequence at least 70%, at least 75%, at least 80%, at least 90%, at least 95%, at least 96%, at least 97%, at least 98%, or at least 99% identical to SEQ ID NO:9. In some embodiments, the nucleic acid is SEQ ID NO:9. In some embodiments, the STC-1 has been codon optimized for expression from an AAV vector. In some embodiments, the STC-1 has been codon optimized for expression in a human ocular cell.

In some embodiments, the nucleic acid encodes an STC-1 propeptide having an amino acid sequence of SEQ ID NO:10, or an amino acid sequence at least 70%, at least 75%, at least 80%, at least 90%, at least 95%, at least 96%, at least 97%, at least 98%, or at least 99% identical to SEQ ID NO:10. In some embodiments, the nucleic acid encodes an STC-1 propeptide having an amino acid sequence of SEQ ID NO:10.

In some embodiments, the nucleic acid is derived from nucleic acid sequence of SEQ ID NO:11, or a nucleic acid sequence at least 70%, at least 75%, at least 80%, at least 90%, at least 95%, at least 96%, at least 97%, at least 98%, or at least 99% identical to SEQ ID NO:11. In some embodiments, the nucleic acid is SEQ ID NO:11. In some embodiments, the STC-1 has been codon optimized for expression from an AAV vector. In some embodiments, the STC-1 has been codon optimized for expression in a human ocular cell.

In some embodiments, the nucleic acid encodes an STC-chain polypeptide having an amino acid sequence of SEQ ID NO:12, or an amino acid sequence at least 70%, at least 75%, at least 80%, at least 90%, at least 95%, at least 96%, at least 97%, at least 98%, or at least 99% identical to SEQ ID NO:12. In some embodiments, the nucleic acid encodes an STC-1 chain polypeptide having an amino acid sequence of SEQ ID NO:12.

In some embodiments, the nucleic acid is derived from the nucleic acid encoding STC-1 isoform 2 (SEQ ID NO:13), or a nucleic acid sequence at least 70%, at least 75%, at least 80%, at least 90%, at least 95%, at least 96%, at least 97%, at least 98%, or at least 99% identical to SEQ ID NO:13. In some embodiments, the nucleic acid is SEQ ID NO:13. In some embodiments, the STC-1 has been codon optimized for expression from an AAV vector. In some embodiments, the STC-1 has been codon optimized for expression in a human ocular cell. In some embodiments, the nucleic acid of SEQ ID NO:13 further comprising a nucleic acid sequence that encodes an amino acid tag.

In some embodiments, the nucleic acid encodes an STC-1 isoform 2 polypeptide having an amino acid sequence of SEQ ID NO:14, or an amino acid sequence at least 70%, at least 75%, at least 80%, at least 90%, at least 95%, at least 96%, at least 97%, at least 98%, or at least 99% identical to SEQ ID NO:14. In some embodiments, the nucleic acid encodes an STC-1 chain polypeptide having an amino acid sequence of SEQ ID NO:14. In some embodiments, the nucleic acid encodes an STC-1 chain polypeptide having an amino acid sequence of SEQ ID NO:14 In some embodiments, the amino acid of SEQ ID NO:14 further comprises an amino acid sequence tag. In some embodiments, the amino acid sequence tag is selected from DYKDDDDK (SEQ ID NO:30), SDYKDDDDK (SEQ ID NO:31), or ASDYKDDDDK (SEQ ID NO:32).

In some embodiments, the STC-1 nucleic acid is derived from the wild-type, consensus, or native STC-1 mRNA sequence of a mammal, for example, Mus musculus, Rat rattus, or a non-human primate. In some embodiments, the nucleic acid is derived from the STC-1 coding sequence of a non-human sequence selected from SEQ ID NO:15, SEQ ID NO:17, SEQ ID NO:19, SEQ ID NO:24, SEQ ID NO:26, or SEQ ID NO:28, or a nucleic acid sequence at least 70%, at least 75%, at least 80%, at least 90%, at least 95%, at least 96%, at least 97%, at least 98%, or at least 99% identical thereto. In some embodiments, the nucleic acid is SEQ ID NO:15. In some embodiments, the nucleic acid is SEQ ID NO:17. In some embodiments, the nucleic acid is SEQ ID NO:19. In some embodiments, the nucleic acid is SEQ ID NO:24. In some embodiments, the nucleic acid is SEQ ID NO:26. In some embodiments, the nucleic acid is SEQ ID NO:28 In some embodiments, the STC-1 has been codon optimized for expression from an AAV vector. In some embodiments, the STC-1 has been codon optimized for expression in a human ocular cell. In some embodiments, the nucleic acid selected from SEQ ID NO:15, SEQ ID NO:17, SEQ ID NO:19, SEQ ID NO:24, SEQ ID NO:26, or SEQ ID NO:28 further comprising a nucleic acid sequence that encodes a polypeptide tag.

In some embodiments, the nucleic acid encodes an STC-1 polypeptide having an amino acid sequence of SEQ ID NO:16, SEQ ID NO:18, SEQ ID NO:20, SEQ ID NO:21, SEQ ID NO:22, SEQ ID NO:23, SEQ ID NO:25, SEQ ID NO:27, or SEQ ID NO:29, or an amino acid sequence at least 70%, at least 75%, at least 80%, at least 90%, at least 95%, at least 96%, at least 97%, at least 98%, or at least 99% identical thereto. In some embodiments, the nucleic acid encodes an STC-1 polypeptide having an amino acid sequence of SEQ ID NO:16. In some embodiments, the nucleic acid encodes an STC-1 polypeptide having an amino acid sequence of SEQ ID NO:2. In some embodiments, the nucleic acid encodes an STC-1 polypeptide having an amino acid sequence of SEQ ID NO:18. In some embodiments, the nucleic acid encodes an STC-1 polypeptide having an amino acid sequence of SEQ ID NO:20. In some embodiments, the nucleic acid encodes an STC-1 polypeptide having an amino acid sequence of SEQ ID NO:21. In some embodiments, the nucleic acid encodes an STC-1 polypeptide having an amino acid sequence of SEQ ID NO:22. In some embodiments, the nucleic acid encodes an STC-1 polypeptide having an amino acid sequence of SEQ ID NO:23. In some embodiments, the nucleic acid encodes an STC-1 polypeptide having an amino acid sequence of SEQ ID NO:25. In some embodiments, the nucleic acid encodes an STC-1 polypeptide having an amino acid sequence of SEQ ID NO:27. In some embodiments, the nucleic acid encodes an STC-1 polypeptide having an amino acid sequence of SEQ ID NO:29. In some embodiments, the amino acid selected from SEQ ID NO:16, SEQ ID NO:18, SEQ ID NO:20, SEQ ID NO:21, SEQ ID NO:22, SEQ ID NO:23, SEQ ID NO:25, SEQ ID NO:27, or SEQ ID NO:29 further comprises a polypeptide tag. In some embodiments, the amino acid sequence tag is selected from DYKDDDDK (SEQ ID NO:30), SDYKDDDDK (SEQ ID NO:31), or ASDYKDDDDK (SEQ ID NO:32).

TABLE 1
STC-1 Nucleic Acid and Amino Acid Sequences.

| SEQ ID NO: | Sequence |
|---|---|
| 1 | ATGCTCCAAAACTCAGCAGTGCTTCTGGTGCTGGTGATCAGTGCTT |
| Nucleic acid | CTGCAACCCATGAGGCGGAGCAGAATGACTCTGTGAGCCCCAGGA |
| sequence- | AATCCCGAGTGGCGGCTCAAAACTCAGCTGAAGTGGTTCGTTGCCT |
| wild-type | CAACAGTGCTCTACAGGTCGGCTGCGGGGCTTTTGCATGCCTGGAA |
| human STC-1 | AACTCCACCTGTGACACAGATGGGATGTATGACATCTGTAAATCCT |
| | TCTTGTACAGCGCTGCTAAATTTGACACTCAGGGAAAAGCATTCGT |
| | CAAAGAGAGCTTAAAATGCATCGCCAACGGGGTCACCTCCAAGGT |
| | CTTCCTCGCCATTCGGAGGTGCTCCACTTTCCAAAGGATGATTGCT |
| | GAGGTGCAGGAAGAGTGCTACAGCAAGCTGAATGTGTGCAGCATC |
| | GCCAAGCGGAACCCTGAAGCCATCACTGAGGTCGTCCAGCTGCCC |
| | AATCACTTCTCCAACAGATACTATAACAGACTTGTCCGAAGCCTGC |
| | TGGAATGTGATGAAGACACAGTCAGCACAATCAGAGACAGCCTGA |
| | TGGAGAAAATTGGGCCTAACATGGCCAGCCTCTTCCACATCCTGCA |
| | GACAGACCACTGTGCCCAAACACACCCACGAGCTGACTTCAACAG |
| | GAGACGCACCAATGAGCCGCAGAAGCTGAAAGTCCTCCTCAGGAA |
| | CCTCCGAGGTGAGGAGGACTCTCCCTCCCACATCAAACGCACATCC |
| | CATGAGAGTGCATAA |
| 2-wt | MLQNSAVLLVLVISASATHEAEQNDSVSPRKSRVAAQNSAEVVRCLN |
| hSTC-1 | SALQVGCGAFACLENSTCDTDGMYDICKSFLYSAAKFDTQGKAFVKE |
| amino acid | SLKCIANGVTSKVFLAIRRCSTFQRMIAEVQEECYSKLNVCSIAKRNPE |
| sequence | AITEVVQLPNHFSNRYYNRLVRSLLECDEDTVSTIRDSLMEKIGPNMA |
| | SLFHILQTDHCAQTHPRADFNRRRTNEPQKLKVLLRNLRGEEDSPSHI |
| | KRTSHESA |
| 3-hSTC-1 | ATGCATGAGGCGGAGCAGAATGACTCTGTGAGCCCCAGGAAATCC |
| propeptide | CGAGTGGCGGCTCAAAACTCAGCTGAAGTGGTTCGTTGCCTCAAC |
| nucleic acid | AGTGCTCTACAGGTCGGCTGCGGGGCTTTTGCATGCCTGGAAAACT |
| sequence | CCACCTGTGACACAGATGGGATGTATGACATCTGTAAATCCTTCTT |
| | GTACAGCGCTGCTAAATTTGACACTCAGGGAAAAGCATTCGTCAA |
| | AGAGAGCTTAAAATGCATCGCCAACGGGGTCACCTCCAAGGTCTT |
| | CCTCGCCATTCGGAGGTGCTCCACTTTCCAAAGGATGATTGCTGAG |
| | GTGCAGGAAGAGTGCTACAGCAAGCTGAATGTGTGCAGCATCGCC |
| | AAGCGGAACCCTGAAGCCATCACTGAGGTCGTCCAGCTGCCCAAT |
| | CACTTCTCCAACAGATACTATAACAGACTTGTCCGAAGCCTGCTGG |
| | AATGTGATGAAGACACAGTCAGCACAATCAGAGACAGCCTGATGG |
| | AGAAAATTGGGCCTAACATGGCCAGCCTCTTCCACATCCTGCAGAC |
| | AGACCACTGTGCCCAAACACACCCACGAGCTGACTTCAACAGGAG |
| | ACGCACCAATGAGCCGCAGAAGCTGAAAGTCCTCCTCAGGAACCT |
| | CCGAGGTGAGGAGGACTCTCCCTCCCACATCAAACGCACATCCCA |
| | TGAGAGTGCATAA |
| 4-hSTC-1 | MHEAEQNDSVSPRKSRVAAQNSAEVVRCLNSALQVGCGAFACLENS |
| propeptide | TCDTDGMYDICKSFLYSAAKFDTQGKAFVKESLKCIANGVTSKVFLAI |
| amino acid | RRCSTFQRMIAEVQEECYSKLNVCSIAKRNPEAITEVVQLPNHESNRY |
| sequence | YNRLVRSLLECDEDTVSTIRDSLMEKIGPNMASLFHILQTDHCAQTHP |
| | RADFNRRRTNEPQKLKVLLRNLRGEEDSPSHIKRTSHESA |
| 5-hSTC-1 | ATGGTGGCGGCTCAAAACTCAGCTGAAGTGGTTCGTTGCCTCAACA |
| chain | GTGCTCTACAGGTCGGCTGCGGGGCTTTTGCATGCCTGGAAAACTC |
| nucleic acid | CACCTGTGACACAGATGGGATGTATGACATCTGTAAATCCTTCTTG |
| sequence | TACAGCGCTGCTAAATTTGACACTCAGGGAAAAGCATTCGTCAAA |
| | GAGAGCTTAAAATGCATCGCCAACGGGGTCACCTCCAAGGTCTTC |
| | CTCGCCATTCGGAGGTGCTCCACTTTCCAAAGGATGATTGCTGAGG |
| | TGCAGGAAGAGTGCTACAGCAAGCTGAATGTGTGCAGCATCGCCA |
| | AGCGGAACCCTGAAGCCATCACTGAGGTCGTCCAGCTGCCCAATC |
| | ACTTCTCCAACAGATACTATAACAGACTTGTCCGAAGCCTGCTGGA |
| | ATGTGATGAAGACACAGTCAGCACAATCAGAGACAGCCTGATGGA |
| | GAAAATTGGGCCTAACATGGCCAGCCTCTTCCACATCCTGCAGACA |
| | GACCACTGTGCCCAAACACACCCACGAGCTGACTTCAACAGGAGA |
| | CGCACCAATGAGCCGCAGAAGCTGAAAGTCCTCCTCAGGAACCTC |
| | CGAGGTGAGGAGGACTCTCCCTCCCACATCAAACGCACATCCCAT |
| | GAGAGTGCATAA |
| 6-hSTC-1 | MVAAQNSAEVVRCLNSALQVGCGAFACLENSTCDTDGMYDICKSFL |
| chain amino | YSAAKFDTQGKAFVKESLKCIANGVTSKVFLAIRRCSTFQRMIAEVQE |
| acid | ECYSKLNVCSIAKRNPEAITEVVQLPNHFSNRYYNRLVRSLLECDEDT |
| sequence | VSTIRDSLMEKIGPNMASLFHILQTDHCAQTHPRADFNRRRTNEPQKL |
| | KVLLRNLRGEEDSPSHIKRTSHESA |
| 7-hSTC-1 | ATGCTCCAAAACTCAGCAGTGCTTCTGGTGCTGGTGATCAGTGCTT |
| hSTC-1 with | CTGCAACCCATGAGGCGGAGCAGAATGACTCTGTGAGCCCCAGGA |
| FLAG-tag | AATCCCGAGTGGCGGCTCAAAACTCAGCTGAAGTGGTTCGTTGCCT |
| nucleic acid | CAACAGTGCTCTACAGGTCGGCTGCGGGGCTTTTGCATGCCTGGAA |
| sequence | AACTCCACCTGTGACACAGATGGGATGTATGACATCTGTAAATCCT |
| | TCTTGTACAGCGCTGCTAAATTTGACACTCAGGGAAAAGCATTCGT |
| | CAAAGAGAGCTTAAAATGCATCGCCAACGGGGTCACCTCCAAGGT |
| | CTTCCTCGCCATTCGGAGGTGCTCCACTTTCCAAAGGATGATTGCT |
| | GAGGTGCAGGAAGAGTGCTACAGCAAGCTGAATGTGTGCAGCATC |
| | GCCAAGCGGAACCCTGAAGCCATCACTGAGGTCGTCCAGCTGCCC |
| | AATCACTTCTCCAACAGATACTATAACAGACTTGTCCGAAGCCTGC |
| | TGGAATGTGATGAAGACACAGTCAGCACAATCAGAGACAGCCTGA |
| | TGGAGAAAATTGGGCCTAACATGGCCAGCCTCTTCCACATCCTGCA |
| | GACAGACCACTGTGCCCAAACACACCCACGAGCTGACTTCAACAG |
| | GAGACGCACCAATGAGCCGCAGAAGCTGAAAGTCCTCCTCAGGAA |
| | CCTCCGAGGTGAGGAGGACTCTCCCTCCCACATCAAACGCACATCC |
| | CATGAGAGTGCAAGCGACTACAAGGACGACGATGACAAGTAA |
| 8-hSTC-1 | MLQNSAVLLVLVISASATHEAEQNDSVSPRKSRVAAQNSAEVVRCLN |
| with FLAG- | SALQVGCGAFACLENSTCDTDGMYDICKSFLYSAAKFDTQGKAFVKE |
| tag amino | SLKCIANGVTSKVFLAIRRCSTFQRMIAEVQEECYSKLNVCSIAKRNPE |
| acid | AITEVVQLPNHFSNRYYNRLVRSLLECDEDTVSTIRDSLMEKIGPNMA |
| sequence | SLFHILQTDHCAQTHPRADFNRRRTNEPQKLKVLLRNLRGEEDSPSHI |
| | KRTSHESASDYKDDDDK |
| 9-hSTC-1 | ATGGTGGCGGCTCAAAACTCAGCTGAAGTGGTTCGTTGCCTCAACA |
| propeptide | GTGCTCTACAGGTCGGCTGCGGGGCTTTTGCATGCCTGGAAAACTC |
| with FLAG- | CACCTGTGACACAGATGGGATGTATGACATCTGTAAATCCTTCTTG |
| tag nucleic | TACAGCGCTGCTAAATTTGACACTCAGGGAAAAGCATTCGTCAAA |
| acid | GAGAGCTTAAAATGCATCGCCAACGGGGTCACCTCCAAGGTCTTC |
| sequence | CTCGCCATTCGGAGGTGCTCCACTTTCCAAAGGATGATTGCTGAGG |
| | TGCAGGAAGAGTGCTACAGCAAGCTGAATGTGTGCAGCATCGCCA |
| | AGCGGAACCCTGAAGCCATCACTGAGGTCGTCCAGCTGCCCAATC |
| | ACTTCTCCAACAGATACTATAACAGACTTGTCCGAAGCCTGCTGGA |
| | ATGTGATGAAGACACAGTCAGCACAATCAGAGACAGCCTGATGGA |
| | GAAAATTGGGCCTAACATGGCCAGCCTCTTCCACATCCTGCAGACA |
| | GACCACTGTGCCCAAACACACCCACGAGCTGACTTCAACAGGAGA |
| | CGCACCAATGAGCCGCAGAAGCTGAAAGTCCTCCTCAGGAACCTC |
| | CGAGGTGAGGAGGACTCTCCCTCCCACATCAAACGCACATCCCAT |
| | GAGAGTGCAGCAAGCGACTACAAGGACGACGATGACAAGTAA |
| 10-hSTC-1 | MTHEAEQNDSVSPRKSRVAAQNSAEVVRCLNSALQVGCGAFACLEN |
| propeptide | STCDTDGMYDICKSFLYSAAKFDTQGKAFVKESLKCIANGVTSKVFL |
| with FLAG- | AIRRCSTFQRMIAEVQEECYSKLNVCSIAKRNPEAITEVVQLPNHFSNR |
| tag amino | YYNRLVRSLLECDEDTVSTIRDSLMEKIGPNMASLFHILQTDHCAQTH |
| acid | PRADFNRRRTNEPQKLKVLLRNLRGEEDSPSHIKRTSHESAASDYKDD |
| sequence | DDK |
| 11-hSTC-1 | ATGGTGGCGGCTCAAAACTCAGCTGAAGTGGTTCGTTGCCTCAACA |
| chain with | GTGCTCTACAGGTCGGCTGCGGGGCTTTTGCATGCCTGGAAAACTC |
| FLAG-tag | CACCTGTGACACAGATGGGATGTATGACATCTGTAAATCCTTCTTG |
| nucleic acid | TACAGCGCTGCTAAATTTGACACTCAGGGAAAAGCATTCGTCAAA |
| sequence | GAGAGCTTAAAATGCATCGCCAACGGGGTCACCTCCAAGGTCTTC |
| | CTCGCCATTCGGAGGTGCTCCACTTTCCAAAGGATGATTGCTGAGG |
| | TGCAGGAAGAGTGCTACAGCAAGCTGAATGTGTGCAGCATCGCCA |
| | AGCGGAACCCTGAAGCCATCACTGAGGTCGTCCAGCTGCCCAATC |
| | ACTTCTCCAACAGATACTATAACAGACTTGTCCGAAGCCTGCTGGA |
| | ATGTGATGAAGACACAGTCAGCACAATCAGAGACAGCCTGATGGA |
| | GAAAATTGGGCCTAACATGGCCAGCCTCTTCCACATCCTGCAGACA |
| | GACCACTGTGCCCAAACACACCCACGAGCTGACTTCAACAGGAGA |
| | CGCACCAATGAGCCGCAGAAGCTGAAAGTCCTCCTCAGGAACCTC |
| | CGAGGTGAGGAGGACTCTCCCTCCCACATCAAACGCACATCCCAT |
| | GAGAGTGCAAGCGACTACAAGGACGACGATGACAAGTAA |
| 12-hSTC-1 | MVAAQNSAEVVRCLNSALQVGCGAFACLENSTCDTDGMYDICKSFL |
| chain with | YSAAKFDTQGKAFVKESLKCIANGVTSKVFLAIRRCSTFQRMIAEVQE |
| FLAG-tag | ECYSKLNVCSIAKRNPEAITEVVQLPNHFSNRYYNRLVRSLLECDEDT |
| amino acid | VSTIRDSLMEKIGPNMASLFHILQTDHCAQTHPRADFNRRRTNEPQKL |
| sequence | KVLLRNLRGEEDSPSHIKRTSHESASDYKDDDDK |
| 13-hSTC-1 | ATGTATGACATCTGTAAATCCTTCTTGTACAGCGCTGCTAAATTTG |
| isoform 2 | ACACTCAGGGAAAAGCATTCGTCAAAGAGAGCTTAAAATGCATCG |
| nucleic acid | CCAACGGGGTCACCTCCAAGGTCTTCCTCGCCATTCGGAGGTGCTC |
| sequence | CACTTTCCAAAGGATGATTGCTGAGGTGCAGGAAGAGTGCTACAG |
| | CAAGCTGAATGTGTGCAGCATCGCCAAGCGGAACCCTGAAGCCAT |
| | CACTGAGGTCGTCCAGCTGCCCAATCACTTCTCCAACAGATACTAT |
| | AACAGACTTGTCCGAAGCCTGCTGGAATGTGATGAAGACACAGTC |
| | AGCACAATCAGAGACAGCCTGATGGAGAAAATTGGGCCTAACATG |
| | GCCAGCCTCTTCCACATCCTGCAGACAGACCACTGTGCCCAAACAC |
| | ACCCACGAGCTGACTTCAACAGGAGACGCACCAATGAGCCGCAGA |
| | AGCTGAAAGTCCTCCTCAGGAACCTCCGAGGTGAGGAGGACTCTC |
| | CCTCCCACATCAAACGCACATCCCATGAGAGTGCATAA |
| 14-hSTC-1 | MYDICKSFLYSAAKFDTQGKAFVKESLKCIANGVTSKVFLAIRRCSTF |
| isoform 2 | QRMIAEVQEECYSKLNVCSIAKRNPEAITEVVQLPNHFSNRYYNRLVR |
| amino acid | SLLECDEDTVSTIRDSLMEKIGPNMASLFHILQTDHCAQTHPRADENR |
| sequence | RRTNEPQKLKVLLRNLRGEEDSPSHIKRTSHESA |
| 15-Mus | atgctccaaaactcagcagtgattctggcgctggtcatcagtgcagctgcagcgc |
| musculus | acgaggcggaacaaaatgattctgtgagccccagaaaatcccgggtggcggctca |
| STC-1 | aaattcagctgaagtggttcgctgcctcaacagtgccctgcaggttggctgcggg |
| nucleic acid | gcttttgcatgcctggaaaactccacatgtgacacagatgggatgtacgacattt |
| sequence | gtaaatccttcttgtacagtgctgctaaatttgacactcagggaaaagcatttgt |
| | caaagagagcttaaagtgcatcgccaatgggatcacctccaaggtattccttgcc |
| | attcggaggtgttcgactttccagaggatgatcgccgaggtgcaggaggactgct |
| | acagcaagcttaacgtttgcagcatcgccaagcgcaacccggaagccatcactga |
| | agtcatacagctgcccaatcacttctccaacagatactacaacagacttgtccga |
| | agccttctggaatgtgatgaagacacggtcagtacaatcagagacagcctgatgg |
| | agaagatcgggcccaacatggccagcctcttccacatcctgcagacagaccactg |
| | tgcccagacacaccccagagctgacttcaataggaggcgcacaaatgagccacag |
| | aagctgaaagtcctcctcaggaacctccgaggtgagggggactctccctcacaca |
| | tcaaacgcacctcccaagagagtgcgtaa |
| 16-Mus | MLQNSAVILALVISAAAAHEAEQNDSVSPRKSRVAAQNSAEVVRCLN |
| musculus | SALQVGCGAFACLENSTCDTDGMYDICKSFLYSAAKFDTQGKAFVKE |
| STC-1 | SLKCIANGITSKVFLAIRRCSTFQRMIAEVQEDCYSKLNVCSIAKRNPE |
| amino acid | AITEVIQLPNHFSNRYYNRLVRSLLECDEDTVSTIRDSLMEKIGPNMAS |
| sequence | LFHILQTDHCAQTHPRADFNRRRTNEPQKLKVLLRNLRGEGDSPSHIK |
| | RTSQESA |
| 17-Mus | ATGCACGAGGCGGAACAAAATGATTCTGTGAGCCCCAGAAAATCC |
| musculus | CGGGTGGCGGCTCAAAATTCAGCTGAAGTGGTTCGCTGCCTCAAC |
| STC-1 | AGTGCCCTGCAGGTTGGCTGCGGGGCTTTTGCATGCCTGGAAAACT |
| propeptide | CCACATGTGACACAGATGGGATGTACGACATTTGTAAATCCTTCTT |
| nucleic acid | GTACAGTGCTGCTAAATTTGACACTCAGGGAAAAGCATTTGTCAA |
| sequence | AGAGAGCTTAAAGTGCATCGCCAATGGGATCACCTCCAAGGTATT |
| | CCTTGCCATTCGGAGGTGTTCGACTTTCCAGAGGATGATCGCCGAG |
| | GTGCAGGAGGACTGCTACAGCAAGCTTAACGTTTGCAGCATCGCC |
| | AAGCGCAACCCGGAAGCCATCACTGAAGTCATACAGCTGCCCAAT |
| | CACTTCTCCAACAGATACTACAACAGACTTGTCCGAAGCCTTCTGG |
| | AATGTGATGAAGACACGGTCAGTACAATCAGAGACAGCCTGATGG |
| | AGAAGATCGGGCCCAACATGGCCAGCCTCTTCCACATCCTGCAGA |
| | CAGACCACTGTGCCCAGACACACCCCAGAGCTGACTTCAATAGGA |
| | GGCGCACAAATGAGCCACAGAAGCTGAAAGTCCTCCTCAGGAACC |
| | TCCGAGGTGAGGGGGACTCTCCCTCACACATCAAACGCACCTCCC |
| | AAGAGAGTGCGTAA |
| 18-Mus | MHEAEQNDSVSPRKSRVAAQNSAEVVRCLNSALQVGCGAFACLENS |
| musculus | TCDTDGMYDICKSFLYSAAKFDTQGKAFVKESLKCIANGITSKVFLAI |
| STC-1 | RRCSTFQRMIAEVQEDCYSKLNVCSIAKRNPEAITEVIQLPNHFSNRYY |
| propeptide | NRLVRSLLECDEDTVSTIRDSLMEKIGPNMASLFHILQTDHCAQTHPR |
| amino acid | ADFNRRRTNEPQKLKVLLRNLRGEGDSPSHIKRTSQESA |
| sequence | |
| 19-Mus | ATGGTGGCGGCTCAAAATTCAGCTGAAGTGGTTCGCTGCCTCAACA |
| musculus | GTGCCCTGCAGGTTGGCTGCGGGGCTTTTGCATGCCTGGAAAACTC |
| STC-1 chain | CACATGTGACACAGATGGGATGTACGACATTTGTAAATCCTTCTTG |
| nucleic acid | TACAGTGCTGCTAAATTTGACACTCAGGGAAAAGCATTTGTCAAA |
| sequence | GAGAGCTTAAAGTGCATCGCCAATGGGATCACCTCCAAGGTATTC |
| | CTTGCCATTCGGAGGTGTTCGACTTTCCAGAGGATGATCGCCGAGG |
| | TGCAGGAGGACTGCTACAGCAAGCTTAACGTTTGCAGCATCGCCA |
| | AGCGCAACCCGGAAGCCATCACTGAAGTCATACAGCTGCCCAATC |
| | ACTTCTCCAACAGATACTACAACAGACTTGTCCGAAGCCTTCTGGA |
| | ATGTGATGAAGACACGGTCAGTACAATCAGAGACAGCCTGATGGA |
| | GAAGATCGGGCCCAACATGGCCAGCCTCTTCCACATCCTGCAGAC |
| | AGACCACTGTGCCCAGACACACCCCAGAGCTGACTTCAATAGGAG |
| | GCGCACAAATGAGCCACAGAAGCTGAAAGTCCTCCTCAGGAACCT |
| | CCGAGGTGAGGGGGACTCTCCCTCACACATCAAACGCACCTCCCA |
| | AGAGAGTGCGTAA |
| 20-Mus | MVAAQNSAEVVRCLNSALQVGCGAFACLENSTCDTDGMYDICKSFL |
| musculus | YSAAKFDTQGKAFVKESLKCIANGITSKVFLAIRRCSTFQRMIAEVQE |
| STC-1 chain | DCYSKLNVCSIAKRNPEAITEVIQLPNHFSNRYYNRLVRSLLECDEDT |
| amino acid | VSTIRDSLMEKIGPNMASLFHILQTDHCAQTHPRADFNRRRTNEPQKL |
| sequence | KVLLRNLRGEGDSPSHIKRTSQESA |
| 21-Mus | MLQNSAVILALVISAAAAHEAEQNDSVSPRKSRVAAQNSAEVVRCLN |
| musculus | SACRLAAGFACLENSTCDTDGMYDICKSFLYSAAKFDTQGKAFVKES |
| STC-1 | LKCIANGITSKVFLAIRRCSTFQRMIAEVQEDCYSKLNVCSIAKRNPEA |
| amino acid | ITEVIQLPNHFSNRYYNRLVRSLLECDEDTVSTIRDSLMEKIGPNMASL |
| sequence | FHILQTDHCAQTHPRADFNRRRTNEPQKLKVLLRNLRGEGDSPSHIKR |
| | TSQESA |
| 22-Mus | MHEAEQNDSVSPRKSRVAAQNSAEVVRCLNSACRLAAGFACLENST |
| musculus | CDTDGMYDICKSFLYSAAKFDTQGKAFVKESLKCIANGITSKVFLAIR |
| STC-1 | RCSTFQRMIAEVQEDCYSKLNVCSIAKRNPEAITEVIQLPNHFSNRYYN |
| propeptide | RLVRSLLECDEDTVSTIRDSLMEKIGPNMASLFHILQTDHCAQTHPRA |
| amino acid | DFNRRRTNEPQKLKVLLRNLRGEGDSPSHIKRTSQESA |
| sequence | |
| 23-Mus | MVAAQNSAEVVRCLNSACRLAAGFACLENSTCDTDGMYDICKSFLY |
| musculus | SAAKFDTQGKAFVKESLKCIANGITSKVFLAIRRCSTFQRMIAEVQED |
| STC-1 chain | CYSKLNVCSIAKRNPEAITEVIQLPNHFSNRYYNRLVRSLLECDEDTVS |
| amino acid | TIRDSLMEKIGPNMASLFHILQTDHCAQTHPRADFNRRRTNEPQKLKV |
| sequence | LLRNLRGEGDSPSHIKRTSQESA |
| 24-Rat | ATGCTCCAAAACTCAGCAGTGATTCTGGCGCTGGTCATCAGTGCTG |
| rattus | CTGCAGCTCACGAGGCGGAACAGAATGATTCTGTGAGCCCCAGAA |
| norvegicus | AATCCCGGGTGGCGGCTCAAAATTCAGCTGAAGTGGTCCGCTGCCT |
| STC-1 | CAACAGTGCCCTACAGGTTGGCTGTGGGGCTTTTGCATGCCTGGAA |
| nucleic acid | AACTCCACATGTGACACAGATGGGATGTACGACATTTGTAAATCCT |
| sequence | TCTTGTACAGTGCTGCTAAATTTGACACTCAGGGAAAAGCATTTGT |
| | CAAAGAGAGCTTAAAGTGCATCGCCAATGGGATCACCTCCAAGGT |
| | CTTCCTTGCCATTCGGAGGTGTTCTACTTTCCAGAGGATGATCGCC |
| | GAGGTGCAGGAGGACTGCTACAGCAAGCTCAATGTTTGCAGCATT |
| | GCCAAGCGCAACCCGGAAGCCATCACTGAAGTCATACAGCTGCCC |
| | AATCACTTCTCCAACAGATACTACAACAGACTTGTCCGAAGCCTTC |
| | TGGAATGTGATGAAGATACGGTCAGCACAATCAGAGACAGCCTGA |
| | TGGAGAAGATCGGGCCCAACATGGCCAGCCTCTTCCATATCCTGCA |
| | GACAGACCACTGTGCCCAGACACACCCCAGAGCTGACTTCAATAG |
| | GAGGCGCACAAATGAGCCACAGAAGCTGAAAGTCCTCCTCAGGAA |
| | CCTCCGAGGTGAGGGGGATTCTCCCTCACACATCAAACGCACCTCC |
| | CAAGAGAATGCGTAA |
| 25-Rat | MLQNSAVILALVISAAAAHEAEQNDSVSPRKSRVAAQNSAEVVRCLN |
| rattus | SALQVGCGAFACLENSTCDTDGMYDICKSFLYSAAKFDTQGKAFVKE |
| norvegicus | SLKCIANGITSKVFLAIRRCSTFQRMIAEVQEDCYSKLNVCSIAKRNPE |
| STC-1 | AITEVIQLPNHFSNRYYNRLVRSLLECDEDTVSTIRDSLMEKIGPNMAS |
| amino acid | LFHILQTDHCAQTHPRADFNRRRTNEPQKLKVLLRNLRGEGDSPSHIK |
| sequence | RTSQENA |
| 26-Rat | ATGCACGAGGCGGAACAGAATGATTCTGTGAGCCCCAGAAAATCC |
| rattus | CGGGTGGCGGCTCAAAATTCAGCTGAAGTGGTCCGCTGCCTCAAC |
| norvegicus | AGTGCCCTACAGGTTGGCTGTGGGGCTTTTGCATGCCTGGAAAACT |
| STC-1 | CCACATGTGACACAGATGGGATGTACGACATTTGTAAATCCTTCTT |
| propeptide | GTACAGTGCTGCTAAATTTGACACTCAGGGAAAAGCATTTGTCAA |
| nucleic acid | AGAGAGCTTAAAGTGCATCGCCAATGGGATCACCTCCAAGGTCTT |
| sequence | CCTTGCCATTCGGAGGTGTTCTACTTTCCAGAGGATGATCGCCGAG |
| | GTGCAGGAGGACTGCTACAGCAAGCTCAATGTTTGCAGCATTGCC |
| | AAGCGCAACCCGGAAGCCATCACTGAAGTCATACAGCTGCCCAAT |
| | CACTTCTCCAACAGATACTACAACAGACTTGTCCGAAGCCTTCTGG |
| | AATGTGATGAAGATACGGTCAGCACAATCAGAGACAGCCTGATGG |
| | AGAAGATCGGGCCCAACATGGCCAGCCTCTTCCATATCCTGCAGA |
| | CAGACCACTGTGCCCAGACACACCCCAGAGCTGACTTCAATAGGA |
| | GGCGCACAAATGAGCCACAGAAGCTGAAAGTCCTCCTCAGGAACC |
| | TCCGAGGTGAGGGGGATTCTCCCTCACACATCAAACGCACCTCCCA |
| | AGAGAATGCGTAA |
| 27-Rat | MHEAEQNDSVSPRKSRVAAQNSAEVVRCLNSALQVGCGAFACLENS |
| rattus | TCDTDGMYDICKSFLYSAAKFDTQGKAFVKESLKCIANGITSKVFLAI |
| norvegicus | RRCSTFQRMIAEVQEDCYSKLNVCSIAKRNPEAITEVIQLPNHFSNRYY |
| STC-1 | NRLVRSLLECDEDTVSTIRDSLMEKIGPNMASLFHILQTDHCAQTHPR |
| propeptide | ADFNRRRTNEPQKLKVLLRNLRGEGDSPSHIKRTSQENA |
| amino acid | |
| sequence | |
| 28-Rat | ATGGTGGCGGCTCAAAATTCAGCTGAAGTGGTCCGCTGCCTCAAC |
| rattus | AGTGCCCTACAGGTTGGCTGTGGGGCTTTTGCATGCCTGGAAAACT |
| norvegicus | CCACATGTGACACAGATGGGATGTACGACATTTGTAAATCCTTCTT |
| STC-1 chain | GTACAGTGCTGCTAAATTTGACACTCAGGGAAAAGCATTTGTCAA |
| nucleic acid | AGAGAGCTTAAAGTGCATCGCCAATGGGATCACCTCCAAGGTCTT |
| sequence | CCTTGCCATTCGGAGGTGTTCTACTTTCCAGAGGATGATCGCCGAG |
| | GTGCAGGAGGACTGCTACAGCAAGCTCAATGTTTGCAGCATTGCC |
| | AAGCGCAACCCGGAAGCCATCACTGAAGTCATACAGCTGCCCAAT |
| | CACTTCTCCAACAGATACTACAACAGACTTGTCCGAAGCCTTCTGG |
| | AATGTGATGAAGATACGGTCAGCACAATCAGAGACAGCCTGATGG |
| | AGAAGATCGGGCCCAACATGGCCAGCCTCTTCCATATCCTGCAGA |
| | CAGACCACTGTGCCCAGACACACCCCAGAGCTGACTTCAATAGGA |
| | GGCGCACAAATGAGCCACAGAAGCTGAAAGTCCTCCTCAGGAACC |
| | TCCGAGGTGAGGGGGATTCTCCCTCACACATCAAACGCACCTCCCA |
| | AGAGAATGCGTAA |
| 29-Rat | MVAAQNSAEVVRCLNSALQVGCGAFACLENSTCDTDGMYDICKSFL |
| rattus | YSAAKFDTQGKAFVKESLKCIANGITSKVFLAIRRCSTFQRMIAEVQE |
| norvegicus | DCYSKLNVCSIAKRNPEAITEVIQLPNHFSNRYYNRLVRSLLECDEDT |
| STC-1 chain | VSTIRDSLMEKIGPNMASLFHILQTDHCAQTHPRADFNRRRTNEPQKL |
| amino acid | KVLLRNLRGEGDSPSHIKRTSQENA |
| sequence | |

In some embodiments, STC-1 polypeptides and nucleic acids encoding STC-I polypeptides are as described elsewhere (see, e.g., U.S. Pat. No. 9,498,517, incorporated herein by reference).

In some embodiments, a nucleic acid can encode a variant of a STC-1 polypeptide in place of or in addition to a STC-1 polypeptide. A variant of a STC-1 polypeptide can have the amino acid sequence of a naturally-occurring STC-1 polypeptide with one or more (e.g., e.g., one, two, three, four, five, six, seven, eight, nine, ten, or more) amino acid deletions, additions, substitutions, or combinations thereof, provided that the variant retains the function of a naturally-occurring STC-1 polypeptide (e.g., reduction of inflammation).

Any appropriate amino acid residue set forth in the amino acid sequences of Table 1 can be deleted, and any appropriate amino acid residue (e.g., any of the 20 conventional amino acid residues or any other type of amino acid such as ornithine or citrulline) can be added to or substituted within the sequences set forth in Table 1. The majority of naturally occurring amino acids are L-amino acids, and naturally occurring polypeptides are largely comprised of L-amino acids. D-amino acids are the enantiomers of L-amino acids. In some embodiments, a polypeptide provided herein can contain one or more D-amino acids. In some embodiments, a polypeptide can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for the amino acids of Table 1 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Further examples of conservative substitutions that can be made at any appropriate position within the amino acids of Table 1 are set forth in Table 2 below.

TABLE 2
Examples of conservative amino acid substitutions.

| Original | Exemplary | Preferred |
|---|---|---|
| Residue | substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, a variant of a STC-1 polypeptide can be designed to include the amino acid sequences set forth in Table 1 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

In some embodiments, a variant of a STC-1 polypeptide having an amino acid or nucleic acid sequence with at least 70% (e.g., 70%, 75%, 80%, 85%, 86%, 87%, 88%, 89%, 90%, 91%, 92%, 93%, 94%, 95%, 96%, 97%, 98%, or 99.0%) sequence identity to the nucleic acid or amino acid sequence set forth in Table 1, provided that it includes at least one difference (e.g., at least one amino acid addition, deletion, or substitution) with respect to the nucleic acid or amino acid sequences of Table 1, can be used. Percent sequence identity is calculated by determining the number of matched positions in aligned sequences, dividing the number of matched positions by the length of an aligned sequence, and multiplying by 100. A matched position refers to a position in which identical amino acids or nucleic acids occur at the same position in aligned sequences.

The percent sequence identity between a particular nucleic acid or amino acid sequence and a sequence referenced by a particular sequence identification number (e.g., SEQ ID NO:1, SEQ ID NO:2, SEQ ID NO:3, SEQ ID NO:4, etc.) is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq -i c:\seq1.txt-j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:1, SEQ ID NO:2, SEQ ID NO:3, SEQ ID NO:4, etc.), followed by multiplying the resulting value by 100. For example, an amino acid sequence that has 247 matches when aligned with the sequence set forth in SEQ ID NO:2 is 96.5 percent identical to the sequence set forth in SEQ ID NO:8 (i.e., 247÷256×100=96.5%). It is noted that the percent sequence identity value is rounded to the nearest tenth. For example, 75.11, 75.13, and 75.14 is rounded down to 75.1, while 75.15, 75.16, 75.17, 75.18, and 75.19 is rounded up to 75.2. It also is noted that the length value will always be an integer.

A nucleic acid encoding a STC-1 polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a STC-1 polypeptide.

As provided herein, the nucleic acid sequences encoding STC-1 described herein are operably linked to a promoter, as described further below.

Vectors Comprising a Nucleic Acid Encoding STC-1

A nucleic acid encoding a STC-1 polypeptide can be delivered to one or both eyes (e.g., to cells within one or both eyes) of a patient for transient expression of a STC-1 polypeptide or for stable expression of a STC-1 polypeptide. In embodiments where a nucleic acid encoding a STC-1 polypeptide is used for stable expression of a STC-1 polypeptide, the nucleic acid encoding a STC-1 polypeptide can be engineered to integrate into the genome of a cell. A nucleic acid can be engineered to integrate into the genome of a cell using any appropriate method. For example, gene editing techniques (e.g., CRISPR or TALEN gene editing) can be used to integrate a nucleic acid designed to express a STC-1 polypeptide into the genome of a cell.

When a nucleic acid encoding a STC-1 polypeptide is delivered to one or both eyes (e.g., to cells within one or both eyes) of a patient for stable expression of a STC-1 polypeptide, the expression of the STC-1 polypeptide can persist for any appropriate amount of time (e.g., following a single delivery such as a single injection). In some embodiments, expression of a STC-1 polypeptide can be detected within one or both eyes of a patient for greater than about 2 weeks following a single delivery (e.g., a single injection) of a nucleic acid encoding a STC-1 polypeptide. For example, expression of a STC-1 polypeptide can be detected within one or both eyes of a patient for at least 2 weeks, at least 30 days, at least 180 days, at least 1 year, at least 3 years, at least 5 years, at least 8 years, or at least 10 years following a single delivery (e.g., a single injection) of a nucleic acid encoding a STC-1 polypeptide. For example, expression of a STC-1 polypeptide can be detected within one or both eyes of a patient for from about 2 weeks to about 10 years (e.g., from about 2 weeks to about 9 years, from about 2 weeks to about 8 years, from about 2 weeks to about 7 years, from about 2 weeks to about 6 years, from about 2 weeks to about 5 years, from about 2 weeks to about 4 years, from about 2 weeks to about 3 years, from about 2 weeks to about 2 years, from about 2 weeks to about 1 year, from about 2 weeks to about 180 days, from about 2 weeks to about 30 days, from about 30 days to about 9 years, from about 30 days to about 8 years, from about 30 days to about 7 years, from about 30 days to about 6 years, from about 30 days to about 5 years, from about 30 days to about 4 years, from about 30 days to about 3 years, from about 30 days to about 2 years, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 year to about 3 years, from about 2 years to about 4 years, from about 3 years to about 5 years, from about 4 years to about 6 years, from about 5 years to about 7 years, from about 6 years to about 8 years, or from about 7 years to about 9 years) following a single delivery (e.g., a single injection) of a nucleic acid encoding a STC-1 polypeptide. In some embodiments, stable expression of a STC-1 polypeptide can be detected within one or both eyes of a patient following a single injection of a nucleic acid encoding a STC-1 polypeptide for about 2 weeks. In some embodiments, stable expression of a STC-1 polypeptide can be detected within one or both eyes of a patient following a single injection of a nucleic acid encoding a STC-1 polypeptide for about 30 days. In some embodiments, stable expression of a STC-1 polypeptide can be detected within one or both eyes of a patient following a single injection of a nucleic acid encoding a STC-1 polypeptide for about 90 days. In some embodiments, stable expression of a STC-1 polypeptide can be detected within one or both eyes of a patient following a single injection of a nucleic acid encoding a STC-1 polypeptide for about 120 days. In some embodiments, stable expression of a STC-1 polypeptide can be detected within one or both eyes of a patient following a single injection of a nucleic acid encoding a STC-1 polypeptide for about 180 days.

When a vector used to deliver a nucleic acid encoding STC-1 polypeptide to an eye (e.g., to cells within an eye) of a patient is a viral vector, any appropriate viral vector can be used. A viral vector can be derived from a positive-strand virus or a negative-strand virus. A viral vector can be derived from a virus having a single-stranded genome or a virus having a double stranded genome. A viral vector can be derived from a virus with a DNA genome or a RNA genome. In some embodiments, a viral vector can be a chimeric viral vector. In some embodiments, a viral vector can infect dividing cells. In some embodiments, a viral vector can infect non-dividing cells. Examples of virus-based vectors that can be used to deliver a nucleic acid encoding a STC-1 polypeptide to one or both eyes (e.g., to cells within one or both eyes) of a patient include, without limitation, virus-based vectors based on adenoviruses, AAVs, Sendai viruses, retroviruses, and lentiviruses. When a vector used to deliver nucleic acid encoding a STC-1 polypeptide is delivered to one or more eyes using an AAV vector, the AAV vector can include three tyrosine to phenylalanine mutations (e.g., can be a AAV2(Triple Y-F) vector) to enhance transduction. When a vector used to deliver a nucleic acid encoding a STC-1 polypeptide is delivered to one or more eyes using an AAV vector, the AAV vector can be any serotype (e.g., AAV2). In some embodiments, nucleic acid encoding a STC-1 polypeptide can be delivered to one or both eyes (e.g., to cells within one or both eyes) of using a viral vector as described elsewhere (see, e.g., Roddy et al., Exp. Eye Res., 165:175-181 (2017); and Ryals et al., Mol. Vis., 17:1090-102 (2011) incorporated herein by reference).

When a vector used to deliver a nucleic acid encoding a STC-1 polypeptide to one or both eyes (e.g., to cells within one or both eyes) of a patient (e.g., a human) is a non-viral vector, any appropriate non-viral vector can be used. In some embodiments, a non-viral vector can be an expression plasmid (e.g., a cDNA expression vector).

In addition to nucleic acid encoding a STC-1 polypeptide, a vector (e.g., a viral vector or a non-viral vector) can contain one or more regulatory elements operably linked to the nucleic acid encoding a STC-1 polypeptide. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a STC-1 polypeptide. A promoter can be a naturally occurring promoter or a recombinant promoter. A promoter can be constitutive or inducible (e.g., in the presence of tetracycline), and can affect the expression of a nucleic acid encoding a polypeptide in a general or cell/tissue-specific manner (e.g., a SYN1 promoter sequence such as a human SYN1 (hSYN1) promoter sequence).

In some embodiments, the STC-1 encoding nucleic acid is operably linked to a constitutively active promoter. Examples of promoters that can be used to drive expression of a STC-1 polypeptide in cells include, without limitation, a cytomegalovirus (CMV) promoter (Boshart et al. Cell 41(2):521-30(1985); Zolotukhin et al. J Virol. 70(7):4646-54(1996); Zolotukhin et al. Gene Ther 6:973-85(1999), each incorporated herein by reference), a β-actin promoter, for example but not limited to a chicken β-actin (CBA) promoter (Acland et al. Nat Genet 28:92-95(2001); Cideciyan et al. Proc Natl Acad Sci 105(39):15112-7(2008); Haire et al., Invest Ophthalmol Vis Sci 2006 September; 47(9):3745-3753, each incorporated herein by reference) or a human β-actin (hACTB) promoter, cytomegalovirus (CMV) immediate-early enhancer and chicken β-actin (CAG) promoter (Haire et al., Invest Ophthalmol Vis Sci 2006 September; 47(9):3745-3753, incorporated herein by reference), a human elongation factor-1 alpha (hEF-1a) promoter, a phosphoglycerate kinase (PGK) promoter, and a ubiquitin C (UbiC) promoter (Schorpp et al. Nucleic Acids Res. 24(9):1787-8(1996); Lois et al. Science 295(5556):868-72(2002), each incorporated herein by reference). In some embodiments, the promoter may include additional regulatory nucleic acid sequences which increase or modulate expression, for example native exons or chimeric introns. Suitable constitutively active promoters for use in expressing STC-1 in the compositions and methods described herein can be derived from, for example, the nucleic acids described in Table 3 below.

TABLE 3
Exemplary Constitutively Active Promoters

| SEQ ID NO: | Sequence |
|---|---|
| 33- | ACTAGTTATTAATAGTAATCAATTACGGGGTCATTAGTTCATAGCCC |
| Chicken | ATATATGGAGTTCCGCGTTACATAACTTACGGTAAATGGCCCGCCT |
| β-actin | GGCTGACCGCCCAACGACCCCCGCCCATTGACGTCAATAATGACGT |
| promoter | ATGTTCCCATAGTAACGCCAATAGGGACTTTCCATTGACGTCAATG |
| | GGTGGAGTATTTACGGTAAACTGCCCACTTGGCAGTACATCAAGTG |
| | TATCATATGCCAAGTACGCCCCCTATTGACGTCAATGACGGTAAAT |
| | GGCCCGCCTGGCATTATGCCCAGTACATGACCTTATGGGACTTTCCT |
| | ACTTGGCAGTACATCTACGTATTAGTCATCGCTATTACCATGGTCGA |
| | GGTGAGCCCCACGTTCTGCTTCACTCTCCCCATCTCCCCCCCCTCCC |
| | CACCCCCAATTTTGTATTTATTTATTTTTTAATTATTTTGTGCAGCGA |
| | TGGGGGCGGGGGGGGGGGGGGGGCGCGCGCCAGGCGGGGCGGGG |
| | CGGGGCGAGGGGCGGGGCGGGGCGAGGCGGAGAGGTGCGGCGGC |
| | AGCCAATCAGAGCGGCGCGCTCCGAAAGTTTCCTTTTATGGCGAGG |
| | CGGCGGCGGCGGCGGCCCTATAAAAAGCGAAGCGCGCGGCGGGCG |
| | GGGAGTCGCTGCGACGCTGCCTTCGCCCCGTGCCCCGCTCCGCCGC |
| | CGCCTCGCGCCGCCCGCCCCGGCTCTGACTGACCGCGTTACTCCCA |
| | CAGGTGAGCGGGCGGGACGGCCCTTCTCCTCCGGGCTGTAATTAGC |
| | GCTTGGTTTAATGACGGCTTGTTTCTTTTCTGTGGCTGCGTGAAAGC |
| | CTTGAGGGGCTCCGGGAGGGCCCTTTGTGCGGGGGGAGCGGCTCGG |
| | GGGGTGCGTGCGTGTGTGTGTGCGTGGGGAGCGCCGCGTGCGGCTC |
| | CGCGCTGCCCGGCGGCTGTGAGCGCTGCGGGCGCGGCGCGGGGCTT |
| | TGTGCGCTCCGCAGTGTGCGCGAGGGGAGCGCGGCCGGGGGCGGT |
| | GCCCCGCGGTGCGGGGGGGGCTGCGAGGGGAACAAAGGCTGCGTG |
| | CGGGGTGTGTGCGTGGGGGGGTGAGCAGGGGGTGTGGGCGCGTCG |
| | GTCGGGCTGCAACCCCCCCTGCACCCCCCTCCCCGAGTTGCTGAGC |
| | ACGGCCCGGCTTCGGGTGCGGGGCTCCGTACGGGGCGTGGCGCGG |
| | GGCTCGCCGTGCCGGGCGGGGGGTGGCGGCAGGTGGGGGTGCCGG |
| | GCGGGGGGGGCCGCCTCGGGCCGGGGAGGGCTCGGGGGAGGGGC |
| | GCGGCGGCCCCCGGAGCGCCGGCGGCTGTCGAGGCGCGGCGAGCC |
| | GCAGCCATTGCCTTTTATGGTAATCGTGCGAGAGGGCGCAGGGACT |
| | TCCTTTGTCCCAAATCTGTGCGGAGCCGAAATCTGGGAGGCGCCGC |
| | CGCACCCCCTCTAGCGGGCGCGGGGCGAAGCGGTGCGGCGCCGGC |
| | AGGAAGGAAATGGGGGGGGAGGGCCTTCGTGCGTCGCCGCGCCGC |
| | CGTCCCCTTCTCCCTCTCCAGCCTCGGGGCTGTCCGCGGGGGGACG |
| | GCTGCCTTCGGGGGGGACGGGGCAGGGCGGGGTTCGGCTTCTGGCG |
| | TGTGACCGGCGGCTCTAGAGCCTCTGCTAACCATGTTCATGCCTTCT |
| | TCTTTTTCCTACAGCTCCTGGGCAACGTGCTGGTTATTGTGCTGTCT |
| | CATCATTTTGGCAAAGAATTC |
| 34- | ctagttattaatagtaatcaattacggggtcattagttcatagcccatatatggagtt |
| Chicken | ccgcgttacataacttacggtaaatggcccgcctggctgaccgcccaacgacccccgc |
| β-actin | ccattgacgtcaataatgacgtatgttcccatagtaacgccaatagggactttccatt |
| promoter | gacgtcaatgggtggactatttacggtaaactgcccacttggcagtacatcaagtgta |
| | tcatatgccaagtacgccccctattgacgtcaatgacggtaaatggcccgcctggcat |
| | tatgcccagtacatgaccttatgggactttcctacttggcagtacatctacgtattag |
| | tcatcgctattaccatggtcgaggtgagccccacgttctgcttcactctccccatctc |
| | ccccccctccccacccccaattttgtatttatttattttttaattattttgtgcagcg |
| | atgggggcggggggggggggggggcgcgcgccaggcggggggggggggcgaggggggg |
| | gcggggcgaggcggagaggtgcggcggcagccaatcagagcggcgcgctccgaaagtt |
| | tccttttatggcgaggcggcggcggcggcggccctataaaaagcgaagcgcgcggggg |
| | gggagtcgctgcgacgctgccttcgccccgtgccccgctccgccgccgcctcgcgccg |
| | cccgccccggctctgactgaccgcgttactcccacaggtgagcgggcgggacggccct |
| | tctcctccgggctgtaattagcgcttggtttaatgacggcttgtttcttttctgtggc |
| | tgcgtgaaagccttgaggggctccgggagctagagcctctgctaaccatgttcatgcc |
| | ttcttctttttcctacagctcctgggcaacgtgctggttattgtgctgtctcatcatt |
| | ttggcaaagaattcctcgaagatctaggcctgcaggcggccgc |
| 35-small | catggtcgaggtgagccccacgttctgcttcactctccccatctcccccccctcccca |
| Chicken | cccccaattttgtatttatttattttttaattattttgtgcagcgatgggggcggggg |
| β-actin | gggggggggggcgcgcgccaggcggggggggcggggcgaggggcggggcggggcgagg |
| promoter | cggagaggtgcggcggcagccaatcagagcggcgcgctccgaaagtttccttttatgg |
| | cgaggcggcggcggcggcggccctataaaaagcgaagcgcgcggcgggcg |
| 36- | TAGTTATTAATAGTAATCAATTACGGGGTCATTAGTTCATAGCCCAT |
| cytomega- | ATATGGAGTTCCGCGTTACATAACTTACGGTAAATGGCCCGCCTGG |
| lovirus | CTGACCGCCCAACGACCCCCGCCCATTGACGTCAATAATGACGTAT |
| (CMV) | GTTCCCATAGTAACGCCAATAGGGACTTTCCATTGACGTCAATGGG |
| promoter | TGGAGTATTTACGGTAAACTGCCCACTTGGCAGTACATCAAGTGTA |
| | TCATATGCCAAGTACGCCCCCTATTGACGTCAATGACGGTAAATGG |
| | CCCGCCTGGCATTATGCCCAGTACATGACCTTATGGGACTTTCCTAC |
| | TTGGCAGTACATCTACGTATTAGTCATCGCTATTACCATGGTGATGC |
| | GGTTTTGGCAGTACATCAATGGGCGTGGATAGCGGTTTGACTCACG |
| | GGGATTTCCAAGTCTCCACCCCATTGACGTCAATGGGAGTTTGTTTT |
| | GGCACCAAAATCAACGGGACTTTCCAAAATGTCGTAACAACTCCGC |
| | CCCATTGACGCAAATGGGCGGTAGGCGTGTACGGTGGGAGGTCTAT |
| | ATAAGCAGAGCTGGTTTAGTGAACCGTCAGATC |
| 37-SV40 | CTGTGGAATGTGTGTCAGTTAGGGTGTGGAAAGTCCCCAGGCTCCC |
| promoter | CAGCAGGCAGAAGTATGCAAAGCATGCATCTCAATTAGTCAGCAAC |
| | CAGGTGTGGAAAGTCCCCAGGCTCCCCAGCAGGCAGAAGTATGCA |
| | AAGCATGCATCTCAATTAGTCAGCAACCATAGTCCCGCCCCTAACT |
| | CCGCCCATCCCGCCCCTAACTCCGCCCAGTTCCGCCCATTCTCCGCC |
| | CCATGGCTGACTAATTTTTTTTATTTATGCAGAGGCCGAGGCCGCCT |
| | CTGCCTCTGAGCTATTCCAGAAGTAGTGAGGAGGCTTTTTTGGAGG |
| | CCTAGGCTTTTGCAAAAAGCT |
| 38- | GTTCCATGTCCTTATATGGACTCATCTTTGCCTATTGCGACACACAC |
| human | TCAATGAACACCTACTACGCGCTGCAAAGAGCCCCGCAGGCCTGAG |
| β-actin | GTGCCCCCACCTCACCACTCTTCCTATTTTTGTGTAAAAATCCAGCT |
| promoter | TCTTGTCACCACCTCCAAGGAGGGGGAGGAGGAGGAAGGCAGGTT |
| | CCTCTAGGCTGAGCCGAATGCCCCTCTGTGGTCCCACGCCACTGAT |
| | CGCTGCATGCCCACCACCTGGGTACACACAGTCTGTGATTCCCGGA |
| | GCAGAACGGACCCTGCCCACCCGGTCTTGTGTGCTACTCAGTGGAC |
| | AGACCCAAGGCAAGAAAGGGTGACAAGGACAGGGTCTTCCCAGGC |
| | TGGCTTTGAGTTCCTAGCACCGCCCCGCCCCCAATCCTCTGTGGCAC |
| | ATGGAGTCTTGGTCCCCAGAGTCCCCCAGCGGCCTCCAGATGGTCT |
| | GGGAGGGCAGTTCAGCTGTGGCTGCGCATAGCAGACATACAACGG |
| | ACGGTGGGCCCAGACCCAGGCTGTGTAGACCCAGCCCCCCCGCCCC |
| | GCAGTGCCTAGGTCACCCACTAACGCCCCAGGCCTGGTCTTGGCTG |
| | GGCGTGACTGTTACCCTCAAAAGCAGGCAGCTCCAGGGTAAAAGGT |
| | GCCCTGCCCTGTAGAGCCCACCTTCCTTCCCAGGGCTGCGGCTGGG |
| | TAGGTTTGTAGCCTTCATCACGGGCCACCTCCAGCCACTGGACCGC |
| | TGGCCCCTGCCCTGTCCTGGGGAGTGTGGTCCTGCGACTTCTAAGTG |
| | GCCGCAAGCCACCTGACTCCCCCAACACCACACTCTACCTCTCAAG |
| | CCCAGGTCTCTCCCTAGTGACCCACCCAGCACATTTAGCTAGCTGA |
| | GCCCCACAGCCAGAGGTCCTCAGGCCCTGCTTTCAGGGCAGTTGCT |
| | CTGAAGTCGGCAAGGGGGAGTGACTGCCTGGCCACTCCATGCCCTC |
| | CAAGAGCTCCTTCTGCAGGAGCGTACAGAACCCAGGGCCCTGGCAC |
| | CCGTGCAGACCCTGGCCCACCCCACCTGGGCGCTCAGTGCCCAAGA |
| | GATGTCCACACCTAGGATGTCCCGCGGTGGGTGGGGGGCCCGAGA |
| | GACGGGCAGGCCGGGGGCAGGCCTGGCCATGCGGGGCCGAACCGG |
| | GCACTGCCCAGCGTGGGGCGCGGGGGCCACGGCGCGCGCCCCCAG |
| | CCCCCGGGCCCAGCACCCCAAGGCGGCCAACGCCAAAACTCTCCCT |
| | CCTCCTCTTCCT |
| 39- | CAATCTCGCTCTCGCTCTTTTTTTTTTTCGCAAAAGGAGGGGAGAGG |
| human | GGGTAAAAAAATGCTGCACTGTGCGGCGAAGCCGGTGAGTGAGCG |
| elongation | GCGCGGGGCCAATCAGCGTGCGCCGTTCCGAAAGTTGCCTTTTATG |
| factor-1- | GCTCGAGCGGCCGCGGCGGCGCCCTATAAAACCCAGCGGCGCGAC |
| alpha | GCGCCACCACCGCCGAGACCGCGTCCGCCCCGCGAGCACAGAGCC |
| (hEF-1α) | TCGCCTTTGCCGATCCGCCGCCCGTCCACACCCGCCGCCAG |
| promoter | |
| 40-phospho- | TTCTACCGGGTAGGGGAGGCGCTTTTCCCAAGGCAGTCTGGAGCAT |
| glycerate | GCGCTTTAGCAGCCCCGCTGGGCACTTGGCGCTACACAAGTGGCCT |
| kinase | CTGGCCTCGCACACATTCCACATCCACCGGTAGGCGCCAACCGGCT |
| (PGK) | CCGTTCTTTGGTGGCCCCTTCGCGCCACCTTCTACTCCTCCCCTAGT |
| promoter | CAGGAAGTTCCCCCCCGCCCCGCAGCTCGCGTCGTGCAGGACGTGA |
| | CAAATGGAAGTAGCACGTCTCACTAGTCTCGTGCAGATGGACAGCA |
| | CCGCTGAGCAATGGAAGCGGGTAGGCCTTTGGGGCAGCGGCCAAT |
| | AGCAGCTTTGCTCCTTCGCTTTCTGGGCTCAGAGGCTGGGAAGGGG |
| | TGGGTCCGGGGGGGGCTCAGGGGGGGGCTCAGGGGCGGGGGGGG |
| | CGCCCGAAGGTCCTCCGGAGGCCCGGCATTCTGCACGCTTCAAAAG |
| | CGCACGTCTGCCGCGCTGTTCTCCTCTTCCTCATCTCCGGGCCTTTC |
| | GACCT |
| 41- | GGTGCAGCGGCCTCCGCGCCGGGTTTTGGCGCCTCCCGCGGGCGCC |
| ubiquitin | CCCCTCCTCACGGCGAGCGCTGCCACGTCAGACGAAGGGCGCAGG |
| C (UbiC) | AGCGTTCCTGATCCTTCCGCCCGGACGCTCAGGACAGCGGCCCGCT |
| promoter | GCTCATAAGACTCGGCCTTAGAACCCCAGTATCAGCAGAAGGACAT |
| | TTTAGGACGGGACTTGGGTGACTCTAGGGCACTGGTTTTCTTTCCAG |
| | AGAGCGGAACAGGCGAGGAAAAGTAGTCCCTTCTCGGCGATTCTGC |
| | GGAGGGATCTCCGTGGGGCGGTGAACGCCGATGATTATATAAGGA |
| | CGCGCCGGGTGTGGCACAGCTAGTTCCGTCGCAGCCGGGATTTGGG |
| | TCGCGGTTCTTGTTTGTGGATCGCTGTGATCGTCACTTGGTGAGTTG |
| | CGGGCTGCTGGGCTGGCCGGGGCTTTCGTGGCCGCCGGGCCGCTCG |
| | GTGGGACGGAAGCGTGTGGAGAGACCGCCAAGGGCTGTAGTCTGG |
| | GTCCGCGAGCAAGGTTGCCCTGAACTGGGGGTTGGGGGGAGCGCA |
| | CAAAATGGCGGCTGTTCCCGAGTCTTGAATGGAAGACGCTTGTAAG |
| | GCGGGCTGTGAGGTCGTTGAAACAAGGTGGGGGGCATGGTGGGCG |
| | GCAAGAACCCAAGGTCTTGAGGCCTTCGCTAATGCGGGAAAGCTCT |
| | TATTCGGGTGAGATGGGCTGGGGCACCATCTGGGGACCCTGACGTG |
| | AAGTTTGTCACTGACTGGAGAACTCGGGTTTGTCGTCTGGTTGCGG |
| | GGGCGGCAGTTATGCGGTGCCGTTGGGCAGTGCACCCGTACCTTTG |
| | GGAGCGCGCGCCTCGTCGTGTCGTGACGTCACCCGTTCTGTTGGCTT |
| | ATAATGCAGGGTGGGGCCACCTGCCGGTAGGTGTGCGGTAGGCTTT |
| | TCTCCGTCGCAGGACGCAGGGTTCGGGCCTAGGGTAGGCTCTCCTG |
| | AATCGACAGGCGCCGGACCTCTGGTGAGGGGAGGGATAAGTGAGG |
| | CGTCAGTTTCTTTGGTCGGTTTTATGTACCTATCTTCTTAAGTAGCTG |
| | AAGCTCCGGTTTTGAACTATGCGCTCGGGGTTGGCGAGTGTGTTTTG |
| | TGAAGTTTTTTAGGCACCTTTTGAAATGTAATCATTTGGGTCAATAT |
| | GTAATTTTCAGTGTTAGACTAGTAAA |

In some embodiments, the nucleic acid encoding an STC-1 polypeptide is operably linked to a promoter derived from a chicken β-actin promoter. In some embodiments, the chicken β-actin promoter is derived from a nucleic acid sequence of SEQ ID NO: 33, or a nucleic acid sequence at least 70%, at least 75%, at least 80%, at least 90%, at least 95%, at least 96%, at least 97%, at least 98%, or at least 99% identical to SEQ ID NO:33. In some embodiments, the chicken β-actin promoter is derived from the nucleic acid sequence of SEQ ID NO: 33. In some embodiments, the chicken β-actin promoter is derived from a nucleic acid sequence of SEQ ID NO: 34, or a nucleic acid sequence at least 70%, at least 75%, at least 80%, at least 90%, at least 95%, at least 96%, at least 97%, at least 98%, or at least 99% identical to SEQ ID NO:34. In some embodiments, the chicken β-actin promoter is derived from the nucleic acid sequence of SEQ ID NO: 34. In some embodiments, the chicken β-actin promoter is derived from a nucleic acid sequence of SEQ ID NO: 35, or a nucleic acid sequence at least 70%, at least 75%, at least 80%, at least 90%, at least 95%, at least 96%, at least 97%, at least 98%, or at least 99% identical to SEQ ID NO:35. In some embodiments, the chicken β-actin promoter is derived from the nucleic acid sequence of SEQ ID NO: 35.

In some embodiments, the nucleic acid encoding an STC-1 polypeptide is operably linked to a promoter derived from a cytomegalovirus (CMV) promoter. In some embodiments, the CMV promoter is derived from a nucleic acid sequence of SEQ ID NO: 36, or a nucleic acid sequence at least 70%, at least 75%, at least 80%, at least 90%, at least 95%, at least 96%, at least 97%, at least 98%, or at least 99% identical to SEQ ID NO:36. In some embodiments, the CMV promoter is derived from the nucleic acid sequence of SEQ ID NO: 36.

In some embodiments, the nucleic acid encoding an STC-1 polypeptide is operably linked to a promoter derived from a SV40 promoter. In some embodiments, the SV40 promoter is derived from a nucleic acid sequence of SEQ ID NO: 37, or a nucleic acid sequence at least 70%, at least 75%, at least 80%, at least 90%, at least 95%, at least 96%, at least 97%, at least 98%, or at least 99% identical to SEQ ID NO:37. In some embodiments, the SV40 promoter is derived from the nucleic acid sequence of SEQ ID NO: 37.

In some embodiments, the nucleic acid encoding an STC-1 polypeptide is operably linked to a promoter derived from a human β-actin promoter. In some embodiments, the human β-actin promoter is derived from a nucleic acid sequence of SEQ ID NO: 38, or a nucleic acid sequence at least 70%, at least 75%, at least 80%, at least 90%, at least 95%, at least 96%, at least 97%, at least 98%, or at least 99% identical to SEQ ID NO:38. In some embodiments, the human β-actin promoter is derived from the nucleic acid sequence of SEQ ID NO: 38.

In some embodiments, the nucleic acid encoding an STC-1 polypeptide is operably linked to a promoter derived from a human elongation factor 1-α (hEF-1a) promoter. In some embodiments, the hEF-1α promoter is derived from a nucleic acid sequence of SEQ ID NO: 39 or a nucleic acid sequence at least 70%, at least 75%, at least 80%, at least 90%, at least 95%, at least 96%, at least 97%, at least 98%, or at least 99% identical to SEQ ID NO:39. In some embodiments, the hEF-1α promoter is derived from the nucleic acid sequence of SEQ ID NO: 39.

In some embodiments, the nucleic acid encoding an STC-1 polypeptide is operably linked to a promoter derived from a phosphoglycerate kinase (PGK) promoter. In some embodiments, the PGK promoter is derived from a nucleic acid sequence of SEQ ID NO: 40 or a nucleic acid sequence at least 70%, at least 75%, at least 80%, at least 90%, at least 95%, at least 96%, at least 97%, at least 98%, or at least 99% identical to SEQ ID NO:40. In some embodiments, the PGK promoter is derived from the nucleic acid sequence of SEQ ID NO: 40.

In some embodiments, the nucleic acid encoding an STC-1 polypeptide is operably linked to a promoter derived from a ubiquitin C (UbiC) promoter. In some embodiments, the UbiC promoter is derived from a nucleic acid sequence of SEQ ID NO: 41 or a nucleic acid sequence at least 70%, at least 75%, at least 80%, at least 90%, at least 95%, at least 96%, at least 97%, at least 98%, or at least 99% identical to SEQ ID NO:41. In some embodiments, the UbiC promoter is derived from the nucleic acid sequence of SEQ ID NO: 41.

In some embodiments, the STC-1 encoding nucleic acid is operably linked to a ocular cell specific promoter. Examples of promoters that can be used to drive expression of a STC-1 polypeptide in cells include, without limitation, a promoter derived from a human synapsin 1 gene (hSYN1) promoter, human rhodopsin (Rho) promoter (see, e.g., Allocca et al. J Virol. 81(20):11372-80(2007); Busskamp et al. Science. 329(5990):413-7(2010), each incorporated herein by reference), human rhodopsin kinase 1 (hRK1) promoter (see, e.g., Khani et al. Invest Ophthalmol Vis Sci. 48(9):3954-61(2007); Boye et al. PLoS One. 5(6):e11306(2010); Boye et al. Hum Gene Ther. 23(10):1101-1115(2012), each incorporated herein by reference), human inter-photoreceptor retinoid binding protein/retinol-binding protein 3 (IRBP/hIRBP241) promoter (see, e.g., Beltran et al. Proc Natl Acad Sci 109(6):2132-7(2012), each incorporated herein by reference), human red opsin (PR2.1/CHOPS2053) promoter (see, e.g., Alexander et al. Nat Med. 13:685-7(2007); Mancuso et al. Nature 461(7265):784-7(2009); Komaromy et al. Hum Mol Genet. 19(13):2581-93(2010), each incorporated herein by reference), hIRBP enhancer fused to cone transducin alpha promoter (IRBP/GNAT2) promoter, human vitelliform macular dystrophy/bestrophin 1 (VMD2/BEST1) promoter (see, e.g., Esumi et al. J Biol Chem. 279(18):19064-73(2004); Deng et al. Invest Ophthalmol Vis Sci. 53(4):1895-904(2012), each incorporated herein by reference), VE-cadherin/Cadherin 5 (CDH5)/CD144 promoter (see, e.g., Cai et al. 2011; Qi et al. 2012, each incorporated herein by reference), Thy 1 promoter (see, e.g., Alic et al. Neurosci Lett. 634:62-41(2016), incorporated herein by reference), neurofilament heavy chain (NEFH) promoter, (see, e.g., Hanlon et al. Front Neurosci. 11:521(2017), incorporated herein by reference), retinal pigmented epithelium 65 (RPE65) promoter, Purkinje cell protein 2 (PCP2) promoter (see, e.g., Korecki et al. Gene Ther. 28(6):351-72(2021), incorporated herein by reference), G Protein Subunit Gamma Transducin 2 (GNGT2) promoter, Phosphodiesterase 6H (PDE6H) promoter (see, e.g., Korecki et al. Gene Ther. 28(6):351-72(2021), incorporated herein by reference), Paired Like Homeodomain 3 (PITX3) promoter (see, e.g., Korecki et al. Gene Ther. 28(6):351-72(2021), incorporated herein by reference), claudin 5 (CLDN5) promoter, Nuclear Receptor Subfamily 2 Group E Member 1 (NR2E1) promoter (see, e.g., Korecki et al. Gene Ther. 28(6):351-72(2021), incorporated herein by reference), paired box 6 (PAX6) promoter (see, e.g., Korecki et al. Gene Ther. 28(6):351-72(2021), incorporated herein by reference), 770En_454 P(hGRM6) promoter (see, e.g., Hulliger et al. Mol Ther Methods Clin Dev. 17:505-19(2020), incorporated herein by reference), or other ocular cell specific promoter described herein. In some embodiments, the ocular cell specific promoter may include additional regulatory nucleic acid sequences which increase or modulate expression, for example native exons, introns, or chimeric introns.

In some embodiments, the nucleic acid encoding an STC-1 polypeptide is operably linked to a promoter derived from a human SYN1 (hSYN1) promoter. In some embodiments, the hSYN1 promoter is derived from a nucleic acid sequence of SEQ ID NO: 42, or a nucleic acid sequence at least 70%, 75%, 80%, 90%, 95%, 96%, 97%, 98%, or 99% identical to SEQ ID NO:42. In some embodiments, the hSYN1 promoter is derived from the nucleic acid sequence of SEQ ID NO: 42. SEQ ID NO:42 is provided in Table 4 below.

In some embodiments, the nucleic acid encoding an STC-1 polypeptide is operably linked to a promoter derived from a human rhodopsin kinase 1 (hRK1) promoter. In some embodiments, the hRK1 promoter is derived from a nucleic acid sequence of SEQ ID NO: 55, or a nucleic acid sequence at least 70%, 75%, 80%, 90%, 95%, 96%, 97%, 98%, or 99% identical to SEQ ID NO:55. In some embodiments, the hRK1 promoter is derived from the nucleic acid sequence of SEQ ID NO: 55. SEQ ID NO:55 is provided in Table 4 below.

In some embodiments, the nucleic acid encoding an STC-1 polypeptide is operably linked to a promoter derived from a fibroblast specific protein 1 (FSP1/S100A4) promoter. In some embodiments, the FSP1/S100A4 promoter is derived from a nucleic acid sequence of SEQ ID NO: 56, or a nucleic acid sequence at least 70%, 75%, 80%, 90%, 95%, 96%, 97%, 98%, or 99% identical to SEQ ID NO:56. In some embodiments, the FSP1/S100A4 promoter is derived from the nucleic acid sequence of SEQ ID NO: 55. SEQ ID NO:55 is provided in Table 4 below.

TABLE 4
Exemplary Cell-Specific Promoter Sequences

| SEQ ID | |
|---|---|
| NO: | Sequence |
| 42- | gatctaggcctactagtctgcagagggccctgcgtatgagtgcaagtgggttttaggaccaggatgaggcggggt |
| hSYN1 | gggggtgcctacctgacgaccgaccccgacccactggacaagcacccaacccccattccccaaattgcgcatcc |
| Promoter | cctatcagagagggggaggggaaacaggatgcggcgaggcgcgtgcgcactgccagcttcagcaccgcgga |
| | cagtgccttcgcccccgcctggcggcgcgcgccaccgccgcctcagcactgaaggcgcgctgacgtcactcgc |
| | cggtcccccgcaaactccccttcccggccaccttggtcgcgtccgcgccgccgccggcccagccggaccgcac |
| | cacgcgaggcgcgagataggggggcacgggcgcgaccatctgcgctgcggcgccggcgactcagcgctgc |
| | ctcagtctgcggtgggcagcggaggagtcgtgtcgtgcctgagagcgcagtcga |
| 54- | GGGCCCCAGAAGCCTGGTGGTTGTTTGTCCTTCTCAGGGGAAAAGTG |
| human | AGGCGGCCCCTTGGAGGAAGGGGCCGGGCAGAATGATCTAATCGGA |
| rhodopsin | TTCCAAGCAGCTCAGGGGATTGTCTTTTTCTAGCACCTTCTTGCCACT |
| kinase 1 | CCTAAGCGTCCTCCGTGACCCCGGCTGGGATTTAGCCTGGTGCTGTG |
| (hRK1) | TCAGCCCCGGTCTCCCAGGGGCTTCCCAGTGGTCCCCAGGAACCCTC |
| promoter | GACAGGGCCCGGTCTCTCTCGTCCAGCAAGGGCAGGGACGGGCCAC |
| | AGGCCAAGGGC |
| 55- | ctacttctaaccctcactgggtttgtagcccaccctgagaggttgacccgaattataactcccctatttcatgccatttc |
| fibroblast | acctctaactctccaccccaacctggattcttcattcctgacactcatcccaactttaaatggcccctcctgataccctc |
| specific | tccgaacctgagatctatccgtgagcccccacgcctcactgccactccactccatcactacctcacccaggaccttt |
| protein 1 | cccactgacgttcctgaggtggtcccagagcctcctttgggtgtgagcctgttcccctccagatccccccgccccg |
| (FSP1/S10 | accctgagccttacttggcatggcagacagtaccgggcatggggatccccaccccagtttttgtttctgaatctttatt |
| 0A4) | tttttaagagacaaggtcctctgtgttgctcaggctggagagcagtggcttgagcatagccaactgcagtctcgaac |
| promoter | tcctgggctcaaatgatcctcctgtctcagcttcctgactagctgggactacaggctacagccatgctgcccagcta |
| | attaaaaaaaaaaattgtttttcctttttatagagacagaagtctctctatgttgcctaggctggtcttgaactcctggc |
| | ctcaggcgatcctcccatctcccccctagcttttgtgtcaccacatttccagggcaatctcccacctgtcacccaccac |
| | cccctgcatctcctttcctaggtccccatgggactactccctgtcccccatgctccaggcacaggctgccccttcctc |
| | cacctctctaaaactcaggctgagctatgtacactgggtggtgcccatctcatccagtcccctgctagtaaccgcta |
| | gggcttacccgttacccacgggtgcccacctgggaacaggaggcttggttccacggctgggctggtggagggt |
| | gctgtggcacttaccgcatcagcccacagcaggaaggcagtatccgctctcccctgtcccctgctatgggcaggg |
| | cctggctggggtataaataggtcagacctctgggccgtccccattcttcccctctctacaaccctctctcctcagcgc |
| | ttcttctttcttggtttggtgagttgtgttggcctgactggcatgcaaggggtgtcagaggccagggctggggaagg |
| | agaaggggaggctggtgggggccagatgtgctaaagagatccagatgtgagattctgatgtggaactctgggtg |
| | gattgtgtgcgtgggtgtgcatggcacacacacacatgcacgtaagacggaggaaaaaacaaacagaaaagtg |
| | agcaagt |

The nucleic acid sequence encoding STC-1 may also include additional regulatory sequences, for example transcription terminating signals, efficient RNA processing signals such as splicing and polyadenylation signals (polyA), sequences that stabilize cytoplasmic mRNA, for example Woodchuck Hepatitis Virus (WHP) Posttranscriptinal Regulatory Element (WPRE), sequences that enhance translation efficiency (ie., Kozak consensus sequence), and/or sequences that enhance protein stability.

In some embodiments, the nucleic acid sequence encoding STC-1 is operably linked to a suitable polyA sequence. Examples of suitable polyA sequences include, but are not limited to, SV40, bovine growth hormone (bGH), and TK polyA. In certain embodiments, the polyA is the bGH poly A sequence. In some embodiments, the bGH poly A sequence is derived from the nucleic acid sequence of SEQ ID NO:43, or a nucleic acid sequence at least 70%, at least 75%, at least 80%, at least 90%, at least 95%, at least 96%, at least 97%, at least 98%, or at least 99% identical to SEQ ID NO:43. In some embodiments, the bGH poly A sequence is derived from the nucleic acid sequence of SEQ ID NO: 43. SEQ ID NO:43 is provided in Table 5 below.

TABLE 5
Exemplary bGH PolyA Sequence

| | |
|---|---|
| SEQ ID | tcgactagagctcgctgatcagcctcgactgtgccttctagtt |
| NO: 43 | gccagccatctgttgtttgcccctcccccgtgccttccttgac |
| | cctggaaggtgccactcccactgtcctttcctaataaaatgag |
| | gaaattgcatcgcattgtctgagtaggtgtcattctattctgg |
| | ggggtggggtggggcaggacagcaagggggaggattgggaaga |
| | caatagcaggcatgctgggga |

In some embodiments, the nucleic acid sequence encoding STC-1 is operably linked to a suitable enhancer. Examples of suitable enhancers include, but are not limited to, the alpha-fetoprotein enhancer, the TTR minimal promoter/enhancer, LSP (TH-binding globulin promoter/alpha 1-microglobulin/bikunin enhancer).

AAV Viral Vectors

In particular aspects, a recombinant adeno-associated virus (rAAV) containing a nucleic acid encoding a STC-1 polypeptide described herein is provided herein for use in the methods of treatment described herein. The rAAV includes an AAV capsid and the nucleic acid encoding STC-1 packaged in the AAV capsid, wherein the nucleic acid encoding STC-1 is operably linked to a promoter as described herein and AAV inverted terminal repeats (ITRs) required for packing the nucleic acid into the capsid. The nucleic acid to be packaged into the AAV capsid contains the STC-1 encoding sequences, promoter sequences, and may include other regulatory sequences described herein, which are flanked by packaging signals of the AAV genome, allowing for the efficient packaging of the nucleic acid into the AAV capsid for delivery and expression in the eye.

The components of the STC-1 encoding nucleic acid are flanked at the 5′ end and the 3′ end by AAV inverted terminal repeat sequences. For example, a 5′ AAV ITR, the nucleic acid sequence encoding an STC-1 polypeptide and operably linked to a promoter and other regulatory sequences, and a 3′ AAV ITR form the nucleic acid that is packaged into the AAV capsid.

The ITRs are the genetic elements responsible for the replication and packaging of the nucleic acid during vector production and are the only viral cis elements required to generate rAAV. In some embodiments, the ITRs are from an AAV different than that supplying a capsid. In some embodiments, the ITR sequences are derived from AAV2, or the deleted version thereof (ΔITR). Where the source of the ITRs is from AAV2 and the AAV capsid is from another AAV source, the resulting vector may be termed pseudotyped. Typically, AAV vector genome comprises an AAV 5′ ITR, the nucleic acid sequences encoding STC-1 and any regulatory sequences, and an AAV 3′ ITR. However, other configurations of these elements may be suitable. In some embodiments, a self-complementary AAV is provided. A shortened version of the 5′ ITR, termed ΔITR, has been described in which the D-sequence and terminal resolution site (TRs) are deleted. In certain embodiments, the nucleic acid for packaging includes a shortened AAV2 ITR of 130 base pairs, wherein the external “a” element is deleted. The shortened ITR is reverted back to the wild-type length during vector DNA amplification using the internal A element as a template. In other embodiments, the full-length AAV 5′ and 3′ ITRs are used. In other embodiments, a full-length or engineered ITR may be selected.

Suitable AAV ITR sequences are known in the art (see, e.g., Yan et al., J Virol. 2005; 79(1):364-379, incorporated herein by reference). Exemplary ITRs suitable for use to package the STC-1 encoding nucleic acid are provided in Table 6 below. In some embodiments, the 5′ ITR is derived from SEQ ID NO:44, SEQ ID NO: 45, SEQ ID NO: 46, SEQ ID NO:56, or a sequence at least 70%, at least 75%, at least 80%, at least 90%, at least 95%, at least 96%, at least 97%, at least 98%, or at least 99% identical thereto. In some embodiments, the 3′ ITR is derived from SEQ ID NO:47, SEQ ID NO: 48, SEQ ID NO: 49, SEQ ID NO:57, or a sequence at least 70%, at least 75%, at least 80%, at least 90%, at least 95%, at least 96%, at least 97%, at least 98%, or at least 99% identical thereto.

TABLE 6
Exemplary ITRs

| SEQ ID | |
|---|---|
| NO: | Sequence |
| 44-5′ ITR | TTGGCCACTCCCTCTCTGCGCGCTCGCTCGCTCACTGAGGCCGGGCG |
| | ACCAAAGGTCGCCCGACGCCCGGGCTTTGCCCGGGCGGCCTCAGTG |
| | AGCGAGCGAGCGCGCAGAGAGGGAGTGGCCAACTCCATCACTAGG |
| | GGTTCCTCAGATCTGAATTCGGT |
| 45-5′ ITR | GGTTGGCCACTCCCTCTCTGCGCGCTCGCTCGCTCACTGAGGCCGGG |
| | CGACCAAAGGTCGCCCGACGCCCGGGCTTTGCCCGGGCGGCCTCAG |
| | TGAGCGAGCGAGCGCGCAGAGAGGGAGTGGCCAACTCCATCACTA |
| | GGGGT |
| 46-5′ ITR | ctgcgcgctcgctcgctcactgaggccgcccgggcaaagcccgggcgtcgggcga |
| | cctttggtcgcccggcctcagtgagcgagcgagcgcgcagagagggagtggccaa |
| | ctccatcactaggggttccttgtagttaatgattaacccgccatgctacttatct |
| | acg |
| 56-5′ ITR | TTGGCCACTCCCTCTCTGCGCGCTCGCTCGCTCACTGAGGCCGGGCG |
| | ACCAAAGGTCGCCCGACGCCCGGGCTTTGCCCGGGCGGCCTCAGTG |
| | AGCGAGCGAGCGCGCAGAGAGGGAGTGGCCAACTCCATCACTAGG |
| | GGTTCCT |
| 47-3′ ITR | AACCCCTAGTGATGGAGTTGGCCACTCCCTCTCTGCGCGCTCGCTCG |
| | CTCACTGAGGCCGCCCGGGCAAAGCCCGGGCGTCGGGCGACCTTTG |
| | GTCGCCCGGCCTCAGTGAGCGAGCGAGCGCGCAGAGAGGGAGTGG |
| | CCAA |
| 48-3′ ITR | AACCCCTAGTGATGGAGTTGGCCACTCCCTCTCTGCGCGCTCGCTCG |
| | CTCACTGAGGCCGCCCGGGCAAAGCCCGGGCGTCGGGCGACCTTTG |
| | GTCGCCCGGCCTCAGTGAGCGAGCGAGCGCGCAGAGAGGGAGTGG |
| | CCAACC |
| 49-3′ ITR | gtagataagtagcatggcgggttaatcattaactacaaggaacccctagtgatgg |
| | agttggccactccctctctgcgcgc |
| | tcgctcgctcactgaggccgggcgaccaaaggtcgcccgacgcccgggctttgcc |
| | cgggcggcctcagtgagcgagcgag cgcgcag |
| 57-3′ ITR | AGGAACCCCTAGTGATGGAGTTGGCCACTCCCTCTCTGCGCGCTCG |
| | CTCGCTCACTGAGGCCGCCCGGGCAAAGCCCGGGCGTCGGGCGACC |
| | TTTGGTCGCCCGGCCTCAGTGAGCGAGCGAGCGCGCAGAGAGGGA |
| | GTGGCCAA |

In some embodiments, the nucleic acid for packaging into an AAV capsid is shown in any of SEQ ID NO:50-53 (Table 7). In some embodiments, the nucleic acid for packaging into an AAV capsid is SEQ ID NO:50, or a sequence at least 70%, at least 75%, at least 80%, at least 90%, at least 95%, at least 96%, at least 97%, at least 98%, or at least 99% identical thereto. In some embodiments, the nucleic acid for packaging into an AAV capsid is SEQ ID NO:50. In some embodiments, a nucleic acid sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:24, SEQ ID NO:26, or SEQ ID NO:28 is substituted for nucleic acids 866-1606 of SEQ ID NO:50.

In some embodiments, the nucleic acid for packaging into an AAV capsid is SEQ ID NO:51, or a sequence at least 70%, at least 75%, at least 80%, at least 90%, at least 95%, at least 96%, at least 97%, at least 98%, or at least 99% identical thereto. In some embodiments, the nucleic acid for packaging into an AAV capsid is SEQ ID NO:51. In some embodiments, a nucleic acid sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:24, SEQ ID NO:26, or SEQ ID NO:28 is substituted for nucleic acids 866-1606 of SEQ ID NO:51.

In some embodiments, the nucleic acid for packaging into an AAV capsid is SEQ ID NO:52, or a sequence at least 70%, at least 75%, at least 80%, at least 90%, at least 95%, at least 96%, at least 97%, at least 98%, or at least 99% identical thereto. In some embodiments, the nucleic acid for packaging into an AAV capsid is SEQ ID NO:52. In some embodiments, a nucleic acid sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:24, SEQ ID NO:26, or SEQ ID NO:28 is substituted for nucleic acids 1143-1883 of SEQ ID NO:52.

In some embodiments, the nucleic acid for packaging into an AAV capsid is SEQ ID NO:53, or a sequence at least 70%, at least 75%, at least 80%, at least 90%, at least 95%, at least 96%, at least 97%, at least 98%, or at least 99% identical thereto. In some embodiments, the nucleic acid for packaging into an AAV capsid is SEQ ID NO:53. In some embodiments, a nucleic acid sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:24, SEQ ID NO:26, or SEQ ID NO:28 is substituted for nucleic acids 1143-1883 of SEQ ID NO:53.

TABLE 7
Exemplary Nucleic Acids for Packaging into AAV

| SEQ ID | | |
|---|---|---|
| NO: | Sequence | Comments |
| 50- | ggttggccactccctctctgcgcgctcgctcgctcactgaggccggg | 5′ ITR 1-143 |
| hSYN- | cgaccaaaggtcgcccgacgcccgggctttgcccgggcggcctca | hSyn1 166-655 |
| hSTC1- | gtgagcgagcgagcgcgcagagagggagtggccaactccatcact | SV40 SD/SA 686-827 |
| FLAG | aggggttcctagatctgaattcggtaccgatctaggcctactagtctg | hSTC1 866-1606 |
| nucleic | cagagggccctgcgtatgagtgcaagtgggttttaggaccaggatg | FLAG 1607-1630 |
| acid | aggcgggggggggtgcctacctgacgaccgaccccgacccactg | bGH Poly A |
| packaging | gacaagcacccaacccccattccccaaattgcgcatcccctatcaga | 1635-1870 |
| insert | gagggggaggggaaacaggatgcggcgaggcgcgtgcgcactg | 3′ ITR 1882-2024 |
| | ccagcttcagcaccgcggacagtgccttcgcccccgcctggcggc | |
| | gcgcgccaccgccgcctcagcactgaaggcgcgctgacgtcactc | |
| | gccggtcccccgcaaactccccttcccggccaccttggtcgcgtcc | |
| | gcgccgccgccggcccagccggaccgcaccacgcgaggcgcga | |
| | gataggggggcacgggcgcgaccatctgcgctgcggcgccggcg | |
| | actcagcgctgcctcagtctgcggtgggcagcggaggagtcgtgtc | |
| | gtgcctgagagcgcagtcgagaattcactctagaggatccggtactc | |
| | gaggaactgaaaaaccagaaagttaactggtaagtttagtctttttgtc | |
| | ttttatttcaggtcccggatccggtggtggtgcaaatcaaagaactgct | |
| | cctcagtggatgttgcctttacttctaggcctgtacggaagtgttacttc | |
| | tgctctaaaagctgcggaattgtacccgcggccgcatgctccaaaac | |
| | tcagcagtgcttctggtgctggtgatcagtgcttctgcaacccatgag | |
| | gcggagcagaatgactctgtgagccccaggaaatcccgagtggcg | |
| | gctcaaaactcagctgaagtggttcgttgcctcaacagtgctctacag | |
| | gtcggctgcggggcttttgcatgcctggaaaactccacctgtgacac | |
| | agatgggatgtatgacatctgtaaatccttcttgtacagcgctgctaaa | |
| | tttgacactcagggaaaagcattcgtcaaagagagcttaaaatgcatc | |
| | gccaacggggtcacctccaaggtcttcctcgccattcggaggtgctc | |
| | cactttccaaaggatgattgctgaggtgcaggaagagtgctacagca | |
| | agctgaatgtgtgcagcatcgccaagcggaaccctgaagccatcac | |
| | tgaggtcgtccagctgcccaatcacttctccaacagatactataacag | |
| | acttgtccgaagcctgctggaatgtgatgaagacacagtcagcacaa | |
| | tcagagacagcctgatggagaaaattgggcctaacatggccagcct | |
| | cttccacatcctgcagacagaccactgtgcccaaacacacccacga | |
| | gctgacttcaacaggagacgcaccaatgagccgcagaagctgaaa | |
| | gtcctcctcaggaacctccgaggtgaggaggactctccctcccacat | |
| | caaacgcacatcccatgagagtgcagactacaaggacgacgatgat | |
| | aagtaagtcgactagagctcgctgatcagcctcgactgtgccttctag | |
| | ttgccagccatctgttgtttgcccctcccccgtgccttccttgaccctgg | |
| | aaggtgccactcccactgtcctttcctaataaaatgaggaaattgcatc | |
| | gcattgtctgagtaggtgtcattctattctggggggggggtggggca | |
| | ggacagcaagggggaggattgggaagacaatagcaggcatgctg | |
| | gggagagatctaggaacccctagtgatggagttggccactccctctc | |
| | tgcgcgctcgctcgctcactgaggccgcccgggcaaagcccgggc | |
| | gtcgggcgacctttggtcgcccggcctcagtgagcgagcgagcgc | |
| | gcagagagggagtggccaacc | |
| 51- | ggttggccactccctctctgcgcgctcgctcgctcactgaggccggg | 5′ ITR 1-143 |
| hSYN- | cgaccaaaggtcgcccgacgcccgggctttgcccgggcggcctca | hSyn1 166-655 |
| hSTC1 | gtgagcgagcgagcgcgcagagagggagtggccaactccatcact | SV40 SD/SA 686-827 |
| nucleic | aggggttcctagatctgaattcggtaccgatctaggcctactagtctg | hSTC1 866-1606 |
| acid | cagagggccctgcgtatgagtgcaagtgggttttaggaccaggatg | bGH Poly A |
| packaging | aggcgggggggggtgcctacctgacgaccgaccccgacccactg | 1611-1847 |
| insert | gacaagcacccaacccccattccccaaattgcgcatcccctatcaga | 3′ ITR 1858-2000 |
| | gagggggaggggaaacaggatgcggcgaggcgcgtgcgcactg | |
| | ccagcttcagcaccgcggacagtgccttcgcccccgcctggcggc | |
| | gcgcgccaccgccgcctcagcactgaaggcgcgctgacgtcactc | |
| | gccggtcccccgcaaactccccttcccggccaccttggtcgcgtcc | |
| | gcgccgccgccggcccagccggaccgcaccacgcgaggcgcga | |
| | gataggggggcacgggcgcgaccatctgcgctgcggcgccggcg | |
| | actcagcgctgcctcagtctgcggtgggcagcggaggagtcgtgtc | |
| | gtgcctgagagcgcagtcgagaattcactctagaggatccggtactc | |
| | gaggaactgaaaaaccagaaagttaactggtaagtttagtctttttgtc | |
| | ttttatttcaggtcccggatccggtggtggtgcaaatcaaagaactgct | |
| | cctcagtggatgttgcctttacttctaggcctgtacggaagtgttacttc | |
| | tgctctaaaagctgcggaattgtacccgcggccgcatgctccaaaac | |
| | tcagcagtgcttctggtgctggtgatcagtgcttctgcaacccatgag | |
| | gcggagcagaatgactctgtgagccccaggaaatcccgagtggcg | |
| | gctcaaaactcagctgaagtggttcgttgcctcaacagtgctctacag | |
| | gtcggctgcggggcttttgcatgcctggaaaactccacctgtgacac | |
| | agatgggatgtatgacatctgtaaatccttcttgtacagcgctgctaaa | |
| | tttgacactcagggaaaagcattcgtcaaagagagcttaaaatgcatc | |
| | gccaacggggtcacctccaaggtcttcctcgccattcggaggtgctc | |
| | cactttccaaaggatgattgctgaggtgcaggaagagtgctacagca | |
| | agctgaatgtgtgcagcatcgccaagcggaaccctgaagccatcac | |
| | tgaggtcgtccagctgcccaatcacttctccaacagatactataacag | |
| | acttgtccgaagcctgctggaatgtgatgaagacacagtcagcacaa | |
| | tcagagacagcctgatggagaaaattgggcctaacatggccagcct | |
| | cttccacatcctgcagacagaccactgtgcccaaacacacccacga | |
| | gctgacttcaacaggagacgcaccaatgagccgcagaagctgaaa | |
| | gtcctcctcaggaacctccgaggtgaggaggactctccctcccacat | |
| | caaacgcacatcccatgagagtgcataagtcgactagagctcgctg | |
| | atcagcctcgactgtgccttctagttgccagccatctgttgtttgcccct | |
| | cccccgtgccttccttgaccctggaaggtgccactcccactgtcctttc | |
| | ctaataaaatgaggaaattgcatcgcattgtctgagtaggtgtcattct | |
| | attctggggggtggggtggggcaggacagcaagggggaggattg | |
| | ggaagacaatagcaggcatgctggggagagatctaggaaccccta | |
| | gtgatggagttggccactccctctctgcgcgctcgctcgctcactgag | |
| | gccgcccgggcaaagcccgggcgtcgggcgacctttggtcgccc | |
| | ggcctcagtgagcgagcgagcgcgcagagagggagtggccaacc | |
| 52- | GGttggccactccctctctgcgcgctcgctcgctcactgaggccgg | 5′ ITR 1-143 |
| CBA- | gcgaccaaaggtcgcccgacgcccgggctttgcccgggcggcctc | CBA promoter526-808 |
| hSTC1- | agtgagcgagcgagcgcgcagagagggagtggccaactccatca | Exon1 809-901 |
| FLAG | ctaggggttcctcagatctgaattcggtaccctagttattaatagtaatc | Chimeric intron |
| nucleic | aattacggggtcattagttcatagcccatatatggagttccgcgttaca | 902-1103 |
| acid | taacttacggtaaatggcccgcctggctgaccgcccaacgaccccc | hSTC1 1143-1883 |
| packaging | gcccattgacgtcaataatgacgtatgttcccatagtaacgccaatag | FLAG-1 |
| insert | ggactttccattgacgtcaatgggtggactatttacggtaaactgccca | 1884-1907 |
| | cttggcagtacatcaagtgtatcatatgccaagtacgccccctattgac | bGH polyA |
| | gtcaatgacggtaaatggcccgcctggcattatgcccagtacatgac | 1933-2147 |
| | cttatgggactttcctacttggcagtacatctacgtattagtcatcgctat | 3′ ITR 2159-2300 |
| | taccatggtcgaggtgagccccacgttctgcttcactctccccatctcc | |
| | cccccctccccacccccaattttgtatttatttattttttaattattttgtgca | |
| | gcgatgggggcggggggggggggggggcgcgcgccaggcgg | |
| | ggcggggcggggcgaggggggggcggggcgaggcggagag | |
| | gtgcggcggcagccaatcagagcggcgcgctccgaaagtttcctttt | |
| | atggcgaggcggcggcggcggcggccctataaaaagcgaagcgc | |
| | gcggcgggcgggagtcgctgcgacgctgccttcgccccgtgcccc | |
| | gctccgccgccgcctcggccgcccgccccggctctgactgaccg | |
| | cgttactcccacaggtgagcggggggacggcccttctcctccggg | |
| | ctgtaattagcgcttggtttaatgacggcttgtttcttttctgtggctgcgt | |
| | gaaagccttgaggggctccgggagctagagcctctgctaaccatgtt | |
| | catgccttcttctttttcctacagctcctgggcaacgtgctggttattgtg | |
| | ctgtctcatcattttggcaaagaattcctcgaagatctaggcctgcagg | |
| | cggccgcatgctccaaaactcagcagtgcttctggtgctggtgatca | |
| | gtgcttctgcaacccatgaggcggagcagaatgactctgtgagccc | |
| | caggaaatcccgagtggcggctcaaaactcagctgaagtggttcgtt | |
| | gcctcaacagtgctctacaggtcggctgcggggcttttgcatgcctg | |
| | gaaaactccacctgtgacacagatgggatgtatgacatctgtaaatcc | |
| | ttcttgtacagcgctgctaaatttgacactcagggaaaagcattcgtca | |
| | aagagagcttaaaatgcatcgccaacggggtcacctccaaggtcttc | |
| | ctcgccattcggaggtgctccactttccaaaggatgattgctgaggtg | |
| | caggaagagtgctacagcaagctgaatgtgtgcagcatcgccaagc | |
| | ggaaccctgaagccatcactgaggtcgtccagctgcccaatcacttc | |
| | tccaacagatactataacagacttgtccgaagcctgctggaatgtgat | |
| | gaagacacagtcagcacaatcagagacagcctgatggagaaaattg | |
| | ggcctaacatggccagcctcttccacatcctgcagacagaccactgt | |
| | gcccaaacacacccacgagctgacttcaacaggagacgcaccaat | |
| | gagccgcagaagctgaaagtcctcctcaggaacctccgaggtgag | |
| | gaggactctccctcccacatcaaacgcacatcccatgagagtgcag | |
| | actacaaggacgacgatgataagtaagtcgactagagctcgctgatc | |
| | agcctcgactgtgccttctagttgccagccatctgttgtttgcccctccc | |
| | ccgtgccttccttgaccctggaaggtgccactcccactgtcctttccta | |
| | ataaaatgaggaaattgcatcgcattgtctgagtaggtgtcattctattc | |
| | tggggggtggggtggggcaggacagcaagggggaggattggga | |
| | agacaatagcaggcatgctggggagagatctgaggaacccctagt | |
| | gatggagttggccactccctctctgcgcgctcgctcgctcactgagg | |
| | ccgcccgggcaaagcccgggcgtcgggcgacctttggtcgcccg | |
| | gcctcagtgagcgagcgagcgcgcagagagggagtggccaa | |
| 53- | GGttggccactccctctctgcgcgctcgctcgctcactgaggccgg | 5′ ITR 1-143 |
| CBA- | gcgaccaaaggtcgcccgacgcccgggctttgcccgggcggcctc | CBA promoter526-808 |
| hSTC1 | agtgagcgagcgagcgcgcagagagggagtggccaactccatca | Exon1 809-901 |
| nucleic | ctaggggttcctcagatctgaattcggtaccctagttattaatagtaatc | Chimeric intron |
| acid | aattacggggtcattagttcatagcccatatatggagttccgcgttaca | 902-1103 |
| packaging | taacttacggtaaatggcccgcctggctgaccgcccaacgaccccc | hSTC1 1143-1883 |
| insert | gcccattgacgtcaataatgacgtatgttcccatagtaacgccaatag | bGH polyA |
| | ggactttccattgacgtcaatgggtggactatttacggtaaactgccca | 1892-2122 |
| | cttggcagtacatcaagtgtatcatatgccaagtacgccccctattgac | 3′ ITR 2135-2276 |
| | gtcaatgacggtaaatggcccgcctggcattatgcccagtacatgac | |
| | cttatgggactttcctacttggcagtacatctacgtattagtcatcgctat | |
| | taccatggtcgaggtgagccccacgttctgcttcactctccccatctcc | |
| | cccccctccccacccccaattttgtatttatttattttttaattattttgtgca | |
| | gcgatgggggcggggggggggggggggcgcgcgccaggcgg | |
| | ggcggggcggggcgaggggggggcggggcgaggcggagag | |
| | gtgcggcggcagccaatcagagcggcgcgctccgaaagtttcctttt | |
| | atggcgaggcggcggcggcggcggccctataaaaagcgaagcgc | |
| | gcggggggggagtcgctgcgacgctgccttcgccccgtgcccc | |
| | gctccgccgccgcctcgcgccgcccgccccggctctgactgaccg | |
| | cgttactcccacaggtgagcggggggacggcccttctcctccggg | |
| | ctgtaattagcgcttggtttaatgacggcttgtttcttttctgtggctgcgt | |
| | gaaagccttgaggggctccgggagctagagcctctgctaaccatgtt | |
| | catgccttcttctttttcctacagctcctgggcaacgtgctggttattgtg | |
| | ctgtctcatcattttggcaaagaattcctcgaagatctaggcctgcagg | |
| | cggccgcatgctccaaaactcagcagtgcttctggtgctggtgatca | |
| | gtgcttctgcaacccatgaggcggagcagaatgactctgtgagccc | |
| | caggaaatcccgagtggcggctcaaaactcagctgaagtggttcgtt | |
| | gcctcaacagtgctctacaggtcggctgcggggcttttgcatgcctg | |
| | gaaaactccacctgtgacacagatgggatgtatgacatctgtaaatcc | |
| | ttcttgtacagcgctgctaaatttgacactcagggaaaagcattcgtca | |
| | aagagagcttaaaatgcatcgccaacggggtcacctccaaggtcttc | |
| | ctcgccattcggaggtgctccactttccaaaggatgattgctgaggtg | |
| | caggaagagtgctacagcaagctgaatgtgtgcagcatcgccaagc | |
| | ggaaccctgaagccatcactgaggtcgtccagctgcccaatcacttc | |
| | tccaacagatactataacagacttgtccgaagcctgctggaatgtgat | |
| | gaagacacagtcagcacaatcagagacagcctgatggagaaaattg | |
| | ggcctaacatggccagcctcttccacatcctgcagacagaccactgt | |
| | gcccaaacacacccacgagctgacttcaacaggagacgcaccaat | |
| | gagccgcagaagctgaaagtcctcctcaggaacctccgaggtgag | |
| | gaggactctccctcccacatcaaacgcacatcccatgagagtgcata | |
| | agtcgactagagctcgctgatcagcctcgactgtgccttctagttgcc | |
| | agccatctgttgtttgcccctcccccgtgccttccttgaccctggaagg | |
| | tgccactcccactgtcctttcctaataaaatgaggaaattgcatcgcatt | |
| | gtctgagtaggtgtcattctattctggggggtggggggggcaggac | |
| | agcaagggggaggattgggaagacaatagcaggcatgctgggga | |
| | gagatctgaggaacccctagtgatggagttggccactccctctctgc | |
| | gcgctcgctcgctcactgaggccgcccgggcaaagcccgggcgtc | |
| | gggcgacctttggtcgcccggcctcagtgagcgagcgagcgcgca | |
| | gagagggagtggccaa | |

The source of the AAV capsid may be a naturally occurring or engineered AAV capsid. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A 1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, WO 2003/042397 (rh.10) and WO 2018/160582 (AAVhu68), each of the above references incorporated herein by reference. These documents also describe other AAV which may be selected for generating AAV and are incorporated by reference.

Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8, AAVAnc80, AAVrh10, and AAVPHP.B and variants of any of the known or mentioned AAVs or AAVs yet to be discovered or variants or mixtures thereof. See, e.g., WO 2005/033321, which is incorporated herein by reference. In one embodiment, the AAV capsid is an AAV1 capsid or variant thereof, AAV8 capsid or variant thereof, an AAV9 capsid or variant thereof.

In some embodiments, the AAV comprises a capsid mutation which assists in delivery. In some embodiments, the AAV comprises a capsid having one or more mutations of the amino acid tyrosine (Y) to phenylalanine (F) on the surface of the capsid protein. In some embodiments, the AAV capsid is an AAV2 capsid comprising one or more mutations of the amino acid tyrosine (Y) to phenylalanine (F) on the surface of the capsid protein. In some embodiments, the AAV2 is selected from AAV2 having a Y444F mutation in its capsid protein, a Y444F and Y730F mutation in its capsid protein, a Y444F and Y500F and Y730F mutation in its capsid protein, a Y272F and Y444F and Y500F and Y730F mutation in its capsid protein, a Y272F and Y444F and Y500F and Y730F and T491V mutation in its capsid protein, and a Y444F and Y500F and Y730F and T491V mutation in its capsid protein. In some embodiments, the AAV capsid is an AAV2 capsid having a Y444F and Y500F and Y730F mutation in its capsid protein. In some embodiments, the AAV capsid is an AAV8 capsid having a Y447F mutation, a Y733F mutation, a Y447F and Y733F mutation, or a Y447F and Y733F and T494V mutation. AAV capsid having tyrosine to phenylalanine mutations are known in the art, as described for example in U.S. Pat. Nos. 8,445,267, 8,802,440, 9,157,098, 9,611,302, 9,775,918, 9,920,097, U.S. Ser. No. 10/011,640, U.S. Ser. No. 10/294,281, U.S. Ser. No. 10/723,768, U.S. Ser. No. 10/815,279, and U.S. Ser. No. 10/934,327, each of which is incorporated by reference.

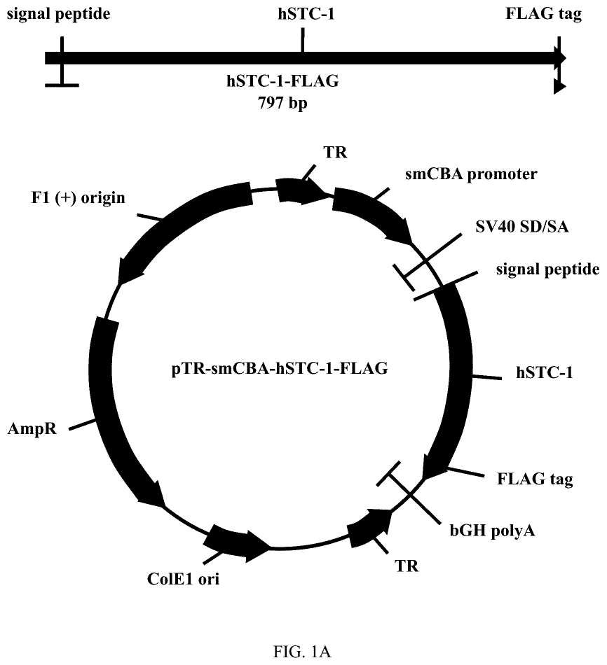

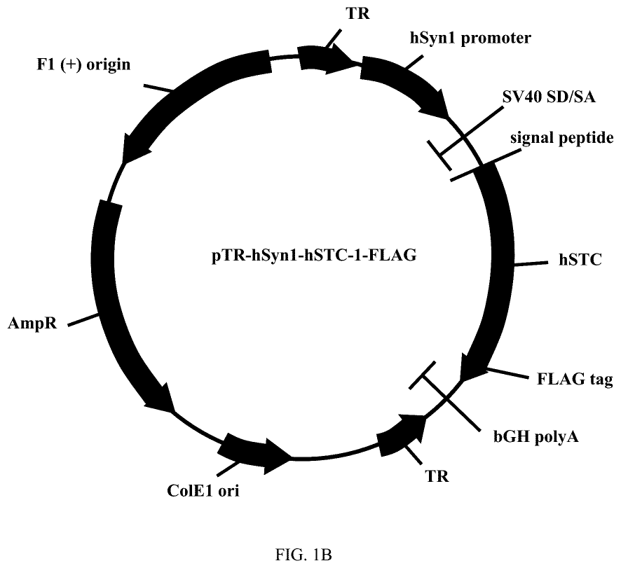

For use in producing an AAV viral vector (e.g., a recombinant (r) AAV), the nucleic acid to be packaged can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art. Exemplary plasmids are described in FIG. 1A and FIG. 1B.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol.99: 119-145; Buning et al., 2008, “Recent developments in adeno-associated virus vector technology,” J. Gene Med.10:717-733; and the references cited below, each of which is incorporated herein by reference in its entirety. For packaging a transgene into virions, the ITRs are the only AAV components required in cis in the same construct as the nucleic acid molecule containing the coding sequence of STC-1. The cap and rep genes can be supplied in trans.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a nucleic acid sequence encoding STC-1; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A.100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding STC-1 operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example, the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293 cells). Methods for manufacturing the rAAV vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and STC-1 nucleic acid coding sequence, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

In some embodiments, a two-step affinity chromatography purification at high salt concentration followed anion exchange resin chromatography are used to purify the vector drug product and to remove empty capsids. These methods are described in more detail in International Patent Publication No. WO 2017/160360, which is incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about ° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods.2014 April; 25(2):115-25. doi:10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

Pharmaceutical Compositions

In some embodiments, one or more nucleic acids designed to express a STC-1 polypeptide can be formulated into a composition (e.g., a pharmaceutical composition) for administration to a patient (e.g., a human). For example, one or more nucleic acids designed to express a STC-1 polypeptide can be formulated into a pharmaceutically acceptable composition for administration to a patient (e.g., a human) having glaucoma. In some embodiments, one or more nucleic acids designed to express a STC-1 polypeptide can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition (e.g., a pharmaceutical composition) containing one or more nucleic acids designed to express a STC-1 polypeptide can be formulated into any appropriate dosage form. Examples of dosage forms include solid or liquid forms including, without limitation, gels, liquids, suspensions, solutions (e.g., sterile solutions), sustained-release formulations, and delayed-release formulations.

A composition (e.g., a pharmaceutical composition) containing one or more nucleic acids designed to express a STC-1 polypeptide can be designed for parenteral (e.g., topical, periocular, and intraocular (e.g., subconjunctival, sub tenon's, intracameral, intravitreal, and subretinal) administration. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets. In some embodiments, compositions designed for topical administration can be prepared as eye drops (e.g., liquid eye drops and gel eye drops).

A composition (e.g., a pharmaceutical composition) containing one or more nucleic acids designed to express a STC-1 polypeptide can be administered locally or systemically. For example, a composition containing one or more nucleic acids designed to express a STC-1 polypeptide can be administered locally by a topical administration to one or both eyes of patient (e.g., a human). For example, a composition containing one or more nucleic acids designed to express a STC-1 polypeptide can be administered locally by an intracameral or subconjunctival injection to one or both eyes of a patient (e.g., a human). In alternative embodiments, a composition containing one or more nucleic acids designed to express a STC-1 polypeptide can be administered locally by subconjunctival injection to one or both eyes of a patient (e.g., a human).

An effective amount (e.g., effective dose) of one or more nucleic acids designed to express a STC-1 polypeptide can vary depending on the severity of the disease or disorder, the route of administration, the age and general health condition of the subject, excipient usage, the possibility of co-usage with other therapeutic treatments such as use of other agents, and/or the judgment of the treating physician.

An effective amount of a composition (e.g., a pharmaceutical composition) containing one or more nucleic acids designed to express a STC-1 polypeptide can be any amount that can treat the patient without producing significant toxicity to the patient. An effective amount of one or more nucleic acids designed to express a STC-1 polypeptide can be any appropriate amount. In embodiments where a viral vector (e.g., an AAV vector) is used to administer nucleic acid designed to express a STC-1 polypeptide, an effective amount of nucleic acid designed to express a STC-1 polypeptide can be from about 1×103 viral genomes per mL (VG/mL) per dose to about 1×1014 VG/mL per dose (e.g., from about 1×103 VG/mL to about 1×1013 VG/mL, from about 1×103 VG/mL to about 1×1012 VG/mL, from about 1×103 VG/mL to about 1×1011 VG/mL, from about 1×103 VG/mL to about 1×1010 VG/mL, from about 1×103 VG/mL to about 1×109 VG/mL, from about 1×103 VG/mL to about 1×108 VG/mL, from about 1×103 VG/mL to about 1×106 VG/mL, from about 1×104 VG/mL to about 1×1014 VG/mL, from about 1×105 VG/mL to about 1×1014 VG/mL, from about 1×106 VG/mL to about 1×1014 VG/mL, from about 1×107 VG/mL to about 1×1014 VG/mL, from about 1×108 VG/mL to about 1×1014 VG/mL, from about 1×109 VG/mL to about 1×1014 VG/mL, from about 1×1010 VG/mL to about 1×1014 VG/mL, from about 1×1011 VG/mL to about 1×1014 VG/mL, from about 1×105 VG/mL to about 1×1010 VG/mL, from about 1×104 VG/mL to about 1×108 VG/mL, or from about 1×108 VG/mL to about 1×1012 VG/mL per dose). For example, an effective amount of nucleic acid designed to express a STC-1 polypeptide can be from about 1×1012 VG/mL to about 1×1013 VG/mL (e.g., about 3.28×1012 VG/mL). The effective amount can remain constant or can be adjusted as a sliding scale or variable dose depending on the patient's response to treatment. Various factors can influence the actual effective amount used for a particular application. For example, the frequency of administration, duration of treatment, use of multiple treatment agents, route of administration, and severity of the condition (e.g., the disease or disorder associated with elevated IOP such as glaucoma) may require an increase or decrease in the actual effective amount administered.

The frequency of administration of a composition (e.g., a pharmaceutical composition) containing one or more nucleic acids designed to express a STC-1 polypeptide can be any frequency that can treat the glaucoma without producing significant toxicity to the patient. For example, the frequency of administration can be from about once a week to about once a month, from about once every two week to once every other month, or from about once a month to about once a year. The frequency of administration can remain constant or can be variable during the duration of treatment. A course of treatment with a composition containing one or more nucleic acids encoding STC-1 provided herein can include rest periods. For example, a composition containing one or more nucleic acids designed to express a STC-1 polypeptide can be administered daily over a six-week period. As with the effective amount, various factors can influence the actual frequency of administration used for a particular application. For example, the effective amount, duration of treatment, use of multiple treatment agents, route of administration, and severity of the condition (e.g., the disease or disorder associated with elevated IOP such as glaucoma) may require an increase or decrease in administration frequency.

An effective duration for administering a composition (e.g., a pharmaceutical composition) containing one or more nucleic acids designed to express a STC-1 polypeptide can be any duration that treat the disease or disorder associated with elevated IOP (e.g., glaucoma) without producing significant toxicity to the patient. For example, the effective duration can vary from several weeks to several months or years. In some embodiments, the effective duration for the treatment of ocular hypertension and/or one or more diseases or disorders associated with elevated IOP (e.g., glaucoma) can range in duration from about one month to about a lifetime. Multiple factors can influence the actual effective duration used for a particular treatment. For example, an effective duration can vary with the frequency of administration, effective amount, use of multiple treatment agents, route of administration, and severity of the condition (e.g., the disease or disorder associated with elevated IOP such as glaucoma) being treated.

Combination Therapy

In some embodiments, the one or more nucleic acids designed to express a STC-1 polypeptide can be used as the sole active agent used to treat a patient having ocular hypertension and/or one or more diseases or disorders associated with elevated IOP (e.g., glaucoma), or a disease or disorder responsive to a reduction in IOP (e.g., NTG).

In some embodiments, the methods and materials described herein can include one or more (e.g., one, two, three, four, five or more) additional therapeutic agents used to treat a patient (e.g., a human) having ocular hypertension and/or one or more diseases or disorders associated with elevated IOP (e.g., glaucoma). In some embodiments, a therapeutic agent used to treat ocular hypertension and/or one or more diseases or disorders associated with elevated IOP can be a prostaglandin. In some embodiments, a therapeutic agent used to treat ocular hypertension and/or one or more diseases or disorders associated with elevated IOP can be an alpha-adrenergic agonist. In some embodiments, a therapeutic agent used to treat ocular hypertension and/or one or more diseases or disorders associated with elevated IOP can be a beta blocker. In some embodiments, a therapeutic agent used to treat ocular hypertension and/or one or more diseases or disorders associated with elevated IOP can be a carbonic anhydrase inhibitor. In some embodiments, a therapeutic agent used to treat ocular hypertension and/or one or more diseases or disorders associated with elevated IOP can be a rho kinase inhibitor. In some embodiments, a therapeutic agent used to treat ocular hypertension and/or one or more diseases or disorders associated with elevated IOP can be a miotic or cholinergic agent. Examples of therapeutic agents used to treat diseases and disorders associated with elevated IOP that can be administered to a patient having ocular hypertension and/or one or more diseases or disorders associated with elevated IOP such as glaucoma together with nucleic acid designed to express a STC-1 polypeptide include, without limitation, latanoprost (XALATAN®), travoprost (TRAVATAN Z®), tafluprost (ZIOPTAN®), bimatoprost (LUMIGAN®), latanoprostene bunod (VYZULTA®), timolol (BETIMOL®, ISTALOL®, and TIMOPTIC®), betaxolol (BETOPTIC®), apraclonidine (IOPIDINE®), brimonidine (ALPHAGAN® P and QOLIANA®), dorzolamide (TRUSOPT®) and brinzolamide (AZOPT®), netarsudil (RHOPRESSA®), pilocarpine (e.g., ISOPTO® CARPINE), timolol-dorzolamide (COS OPT®), brinzolamide/brimonidine tartrate (SIMBRINZAC), brimonidine (COMBIGANC), netarsudil/latanoprost (ROCKLATAN®), methazolamide, acetazolamide (DIAMOX®), and medicinal marijuana. In some embodiments, the one or more additional therapeutic agents can be administered together with one or more nucleic acids designed to express a STC-1 polypeptide (e.g., in the same composition). In some embodiments, the one or more additional therapeutic agents can be administered independent of the one or more nucleic acids designed to express a STC-1 polypeptide. When the one or more additional therapeutic agents are administered independent of the one or more nucleic acids designed to express a STC-1 polypeptide, the one or more nucleic acids designed to express a STC-1 polypeptide can be administered first, and the one or more additional therapeutic agents administered second, or vice versa.

In some embodiments, the methods and materials described herein can include subjecting a patient having ocular hypertension and/or one or more diseases or disorders associated with elevated IOP (e.g., glaucoma) to one or more (e.g., one, two, three, four, five or more) additional treatments (e.g., therapeutic interventions) that are effective to treat glaucoma. Examples of additional treatments that can be used as described herein to treat ocular hypertension and/or one or more diseases or disorders associated with elevated IOP such as glaucoma include, without limitation, laser therapy (e.g., laser trabeculoplasty, and laser peripheral iridotomy), surgery (e.g., a trabeculectomy, implantation of drainage tubes, and minimally invasive glaucoma surgery), lifestyle changes (e.g., increased physical activity, and sleeping with the head elevated), and/or dietary changes (e.g., reduced caffeine consumption, increased hydration, maintaining healthy zinc, copper, selenium, vitamin C, vitamin E, and vitamin A levels). In some embodiments, the one or more additional treatments that are effective to treat ocular hypertension and/or one or more diseases or disorders associated with elevated IOP (e.g., glaucoma) can be performed at the same time as the administration of the one or more nucleic acids designed to express a STC-1 polypeptide. In some embodiments, the one or more additional treatments that are effective to treat glaucoma can be performed before and/or after the administration of the one or more nucleic acids designed to express a STC-1 polypeptide.

The invention will be further described in the following examples, which do not limit the scope of the invention described in the claims.

### EXAMPLES

### Example 1: Sustained Ocular Delivery of STC-1

This Example demonstrates that delivery of a nucleic acid capable of expressing an STC—1 polypeptide using a viral vector provides targeted and long-lasting reduction of IOP.

### Methods

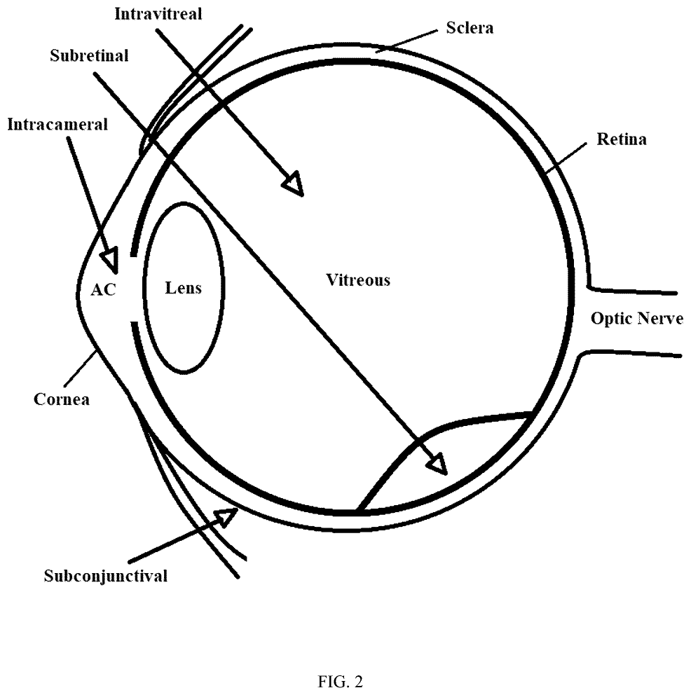

Single strand (ss) AAV2 (Triple Y-F) (Petrs-Silva et al. Mol Ther. 19(2):293-301(2011)) constructs were generated to deliver the human STC-1 coding region (SEQ ID NO:1) encoding an STC-1 polypeptide (SEQ ID NO:2) with the signal peptide and a C-terminal FLAG tag (SEQ ID NO:30) using either the ubiquitously active small chicken β-actin (smCBA) promotor (SEQ ID NO:34)(ssAAV2-smCBA-STC-1-FLAG: packaging insert SEQ ID NO:50)(FIG. 1A) or the retinal ganglion cell (RGC) specific promoter human synapsin 1 (hSyn1, SEQ ID NO:42)(ssAAV2-hSYN1-STC-1-FLAG: package insert SEQ ID NO:52). The constitutively active small CBA (smCBA) promoter is a truncated version of the CBA promoter, which is a fusion of the CBA promoter and the CMV immediate-early cytomegalovirus enhancer (Sawicki et al. Exp Cell Res 244:367-9(1998)). The full-length CBA promoter was truncated by internal sequence removal of 780 base pairs and condensing the hybrid chicken β-actin/rabbit β-globin intron. The smCBA promoter retinal expression pattern is similar to that of the full-length CBA promoter. To drive expression in several tissues the ssAAV2-STC-1-FLAG construct with the smCBA promoter was used for administration to the anterior chamber of the eye by intracameral injection (FIG. 2). Wild-type mice were injected with a single intracameral injection of 1 μL AAV2-STC-1 (3.28E+12 VG/mL), and the same volume of PBS was injected in the fellow eye using a 32 g Hamilton needle and syringe. IOP was measured twice weekly with a handheld rebound tonometer following injection.

### Results

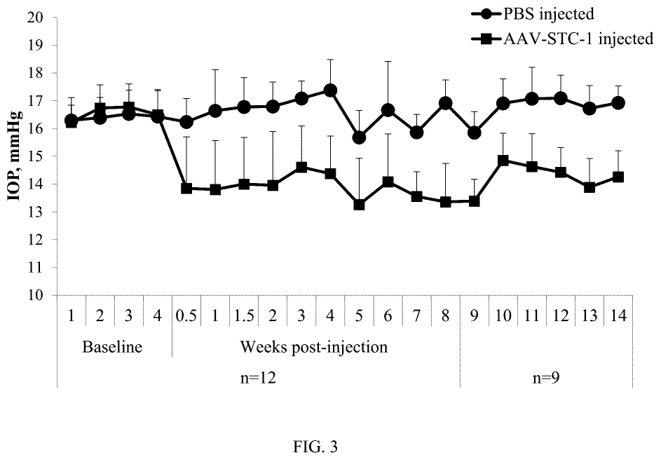

After baseline IOP measurements, ssAAV2-smCBA-STC-1-FLAG was injected in one eye and PBS was injected into the fellow eye of wild-type 3-month-old mice (n=12, 1 μL, intracameral injection). IOP was checked twice weekly for 14 weeks (experiment still ongoing at time of application submission. Sustained IOP reduction of 16.5±2.3% was seen continuously for 98 days (FIG. 3).

These results demonstrate that STC-1 administration can reduce IOP in ocular hypertension.

### Example 2: Targeted and Sustained Reduction of Intraocular Pressure by AAV Expressed Stanniocalcin-1

This Example demonstrates that single or multiple administrations of transgenic STC-1 in the anterior chamber of the mouse eye provides sustainable IOP reduction via increased outflow facility.

### Methods

Mouse Experiments

All animal studies and treatment protocols were pre-approved by the Mayo Clinic (Rochester, MN) Institutional Animal Care and Use Committee and adhered to the ARVO Statement for the Use of Animals in Ophthalmic and Vision Research. All mouse strains had unrestricted access to food and water and were housed with 12 hour alternating light and dark cycles. Mice were humanely euthanized using carbon dioxide asphyxiation followed by cervical dislocation.

Adeno-Associated Viral Vector

AAV2 to express STC-1 with a FLAG tag (ssAAV2-STC-1-FLAG) was generated as previously described (Jacobson et al., Mol Ther. 13:1074-84(2006)) (Example 1). The construct was designed based on prior neuroprotection studies performed using ssAAV2-STC-1-FLAG containing a hSYN1 promoter (SEQ ID NO:42), specific for retinal ganglion cells (Roddy et al., Exp Eye Res. 165:175-81(2017)) (FIG. 1B). For the current studies, the hSYN1 promoter was replaced with the CBA promotor (ssAAV2-smCBA-STC-1-FLAG, SEQ ID NO:34, FIG. 1A) to allow for ubiquitous ocular expression following an intracameral injection. The vector was packaged in a capsid-modified AAV2 vector containing 3 surface-exposed tyrosine to phenylalanine mutations (triple Y-F) (Petrs-Silva et al. Mol Ther. 19(2):293-301(2011)). For a control, an identical vector was generated comprising a nucleic acid encoding green fluorescent protein (GFP; ssAAV2-smCBA-GFP) in place of the STC-1-FLAG transgene.

Intracameral Injection

Three-month-old C57B/6J wild-type or FP receptor knockout mice generated (Roddy et al., PLoS One. 15(2020)) were anesthetized with an intraperitoneal injection of ketamine/xylazine/acepromazine (80/6/1 mg/kg body weight) for intracameral injections. Mice were subsequently placed on a dissecting microscope to visualize the anterior chamber. A 32G beveled needle (Hamilton Company, Reno, NV) containing the AAV2 construct (1 μL; 3×1012 VG/mL) or 1 μL phosphate buffered saline (PBS) was placed parallel to the cornea and inserted anterior to the limbus into the anterior chamber. Care was taken to avoid iris trauma as well as avoidance of the corneal endothelium and anterior lens capsule. The injected volume was administered into the anterior chamber over 2 seconds. The needle was then slowly removed to minimize reflux.

Intraocular Pressure Measurements

IOP was measured in conscious mice using a handheld rebound tonometer (Icare TonoLab; Colonial Medical Supply, Franconia, NH). For measurements of IOP, the conscious mouse was gently restrained in a decapicone (Braintree, MA) and the probe of the Tonolab tonometer was positioned perpendicular and approximately 3 mm from the mouse cornea. Upon initiation, the probe extends out and rebounds off the cornea six times, each time taking a measurement. The internal software discards the highest and lowest values and shows the average of the remaining four values as a single measurement. For each IOP measurement, three sequential but independent readings were obtained, averaged, and recorded. A single laboratory technician measured and recorded all IOPs while a second laboratory member who was masked to treatment groups checked IOP at multiple points throughout the experiment to ensure accurate data collection.

For all experimental protocols, mice underwent 3-4 days of twice daily Tonolab measurements to obtain baseline IOP prior to treatment. These measurements were averaged and taken as a single baseline value. After treatment, IOPs were measured twice daily for up to four times weekly for the duration of each experiment. For summary graphs and statistical analysis, IOP was averaged, taken as a single value, and presented as weekly, bi-monthly, or monthly values.

Evaluation of RNA Expression by ssAAV2-smCBA-STC-1-FLAG

To determine expression of ssAAV2-smCBA-STC-1-FLAG at the gene level, mice were euthanized and eyes were enucleated by surgical excision and immediately placed in RNA isolation reagent (RNA Bee; Tel-Test, Friendswood, TX) and frozen at −80° C. Prior to RNA isolation, samples were removed from the freezer, thawed and homogenized on ice. Following removal of the aqueous phase, total RNA was isolated and purified (RNeasy Mini kit; Qiagen, Valencia, CA). cDNA was generated by reverse transcription using 1 μg total RNA (iScript cDNA synthesis kit, Biorad, Hercules, CA). Oligonucleotides were designed to amplify STC-1-FLAG avoiding amplification of endogenous STC-1 transcripts by designing the forward primer in the STC-1 sequence and the reverse primer in the FLAG tag sequence (Forward primer: 5′-GTGCTTCTGCAACCCATGAGG-3′; Reverse primer: 5′-CTTGTCATCGTCGTCCTTGTAGTCG-3′). PCR reactions were prepared and the following PCR conditions were used: 95° C., 6 min, 1 cycle; 95° C., 1 min, 60° C., 1 min, 68° C., 2.5 min, 40 cycles; 68° C., 7 min, 1 cycle; 4° C. until recovery, 1 cycle. Aliquots of each PCR reaction (15 μL) were separated on a 1% agarose gel.

Assessment of STC-1-FLAG Protein Expression

To confirm expression of STC-1-FLAG at the protein level, immunohistochemistry (IHC) was performed. Mice were euthanized, whole globes were enucleated, fixed in 4% paraformaldehyde in 0.1M phosphate buffer, and embedded in paraffin. Paraffin blocks were sectioned at 5 μm and mounted on poly-L-lysine coated slides. Following deparaffinization, antigen retrieval was performed by placing slides in a 1 mM solution of EDTA pH 8.0 at 95° C. for 30 min. Slides were rinsed twice at room temperature for five minutes each in PBS. Slides were placed in a humidified chamber and sections were blocked with 10% goat serum and 3% albumin in PBS at room temperature for 30 minutes. Following incubation, slides were rinsed with PBS at room temperature twice for 2 minutes. Sections were incubated with anti-FLAG primary antibody (1:100; Sigma, St. Lois, MO) in 1% ovalbumin overnight at 4° C. Slides were rinsed twice in PBS at room temperature for 5 minutes and then incubated with Alexa Fluor 488 antibody (1:200; Abcam) for one hour at room temperature. Vectashield containing DAPI (Vector Laboratories, Burlingame, CA) was added to the slides, followed by placement of a coverslip, and imaged on a Zeiss LSM780 confocal microscope (Zeiss, Dublin, CA).

Assessment of Aqueous Outflow Parameters by Constant Flow Infusion

To determine the effects of ssAAV2-smCBA-STC-1-FLAG on parameters of aqueous outflow, 3-month old C57BL/6J mice were separately treated with the following: 1) intracameral ssAAV2-smCB A-STC-1-FLAG (n=5), 2) intracameral ssAAV2-smCBA-GFP (n=5), 3) topical recombinant human STC-1 (n=4; Biovender Research and Diagnostic Products, Czech Republic) (Roddy et al., Invest Ophthalmol Vis Sci. 58:2715-24(2017); Roddy et al., PLoS ONE. 15(2020)), 4) topical latanoprost free acid (LFA; n=5; Cayman Chemical, Ann Arbor, MI), or 5) untreated mice (n=4). Mice that received topical treatments were treated with once daily topical doses of STC-1 (5 μl of 0.5 μg/μl) or LFA (10-4 M) for 5 days prior to aqueous outflow experiments. Mice that received intracameral injections were treated 6 weeks prior to aqueous outflow measurements. Aqueous outflow was assessed using the protocols previously established (Millar et al., Invest Ophthalmol Vis Sci. 52:685-94(2011); Roy Chowdhury et al., Invest Ophthalmol Vis Sci. 58:5731-42(2017)). Briefly, a 33-g needle was used to cannulate the anterior chamber of anesthetized mice. The needle was connected to a flow-through pressure transducer (World Precision Instruments, Sarasota, FL) that was also connected to an SP101i microdialysis infusion pump (World Precision Instruments) and an open-ended, variable-height, raised reservoir manometer. Episcleral venous pressure was obtained by visualizing the reflux of blood into Schlemm's canal by direct ophthalmoscopy as the reservoir was lowered. Outflow facility was determined by slowly increasing the flow rate with the infusion pump and recording the pressure. Uveoscleral outflow was calculated when the inflow is presumed to be zero immediately after animal sacrifice. Aqueous humor formation was then estimated by using the modified Goldmann equation, i.e. aqueous formation rate=[outflow facility X (IOP−episcleral venous pressure)]+uveoscleral outflow as previously described (Millar et al., Invest Ophthalmol Vis Sci. 52:685-94(2011)). To validate the calculated IOP values based on the Goldman equation, IOP was also assessed with a handheld rebound tonometer. All data were recorded and analyzed using LabScribe software (World Precision Instruments).

### Results

Intraocular Pressure Lowering Response Following Injection with ssAAV2-smCBA-STC-1-FLAG

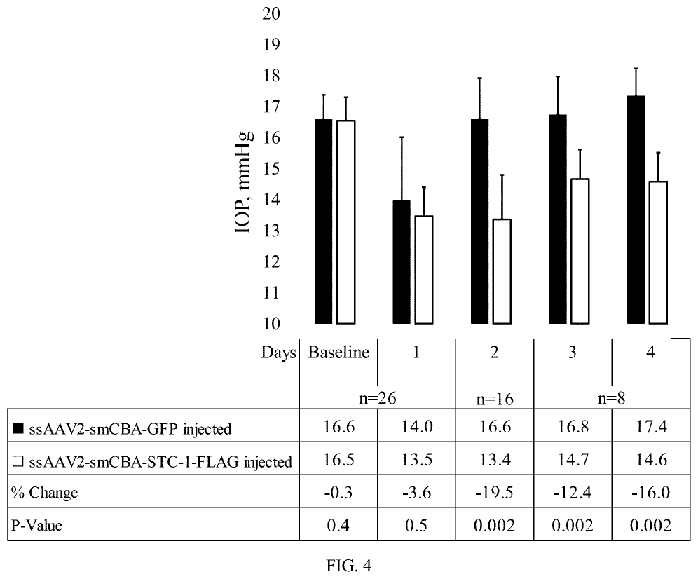

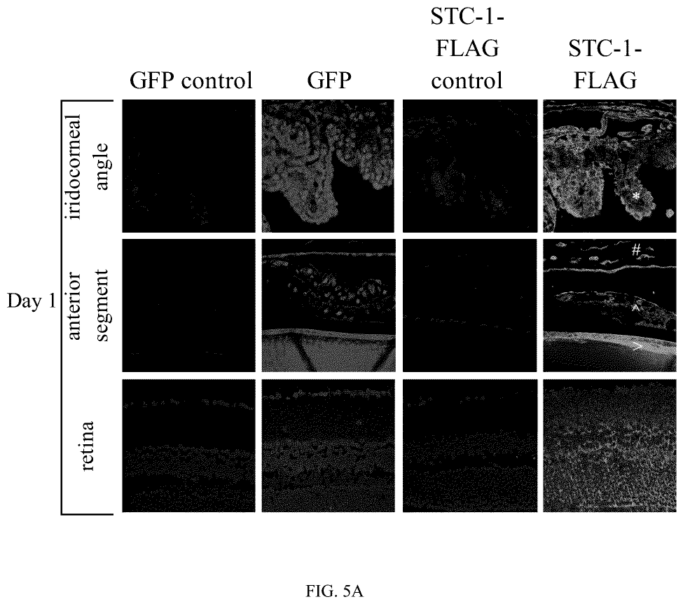

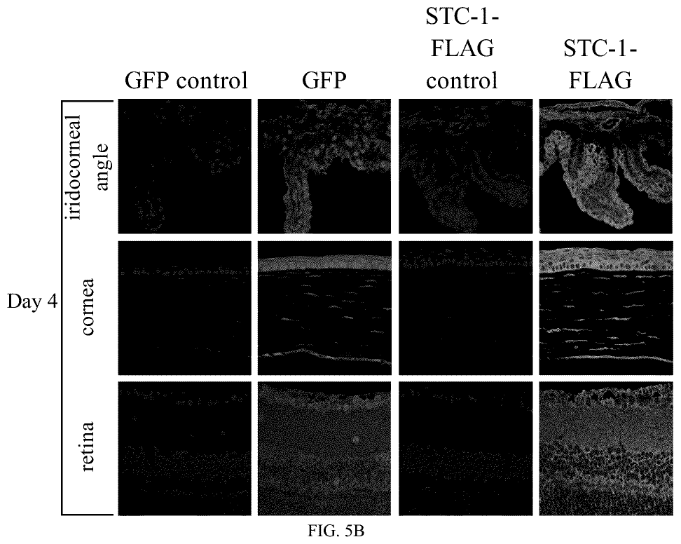

In order to determine whether STC-1-FLAG expressed by an AAV lowered IOP, AAV vectors designed to express a STC-1 polypeptide were administered locally by an intracameral injection to mice as described in Example 1. C57BL/6J mice with no difference in baseline IOP between fellow eyes (16.5±0.8 mmHg vs 16.6±0.8 mmHg, P=0.4, n=26, FIG. 4) received a single intracameral injection of ssAAV2-smCBA-STC-1-FLAG (3×109 VGs) in one eye and ssAAV2-smCBA-GFP (3×109 VGs) in the fellow eye. IOP was assessed daily and tissues were collected at days 1, 2, and 4 for expression analysis. At day one post injection, there was no significant difference in IOP between ssAAV2-smCBA-STC-1-FLAG and ssAAV2-smCBA-GFP-injected animals (13.5±0.9 vs. 14.0±2.0 mmHg, P=0.5, n=26, FIG. 4), although both eyes showed an IOP drop from baseline. On day 2, IOP in the eyes injected with ssAAV2-smCBA-GFP had returned to baseline levels (16.6±1.3 mmHg), but the eyes injected with ssAAV2-smCBA-STC-1-FLAG remained lower (13.4±1.4 mmHg) showing a significant change (P<0.01, n=16, FIG. 4). Immunofluorescence revealed GFP fluorescence (red fluorochrome) as well as STC-1-FLAG expression (green fluorochrome) starting at day 1 in the iridocorneal angle with highest levels of expression in the ciliary body, in the anterior segment with expression in the cornea, iris, and lens capsule, and in the retina (FIG. 5A). Expression in these tissues remained evident until termination of the experiment at day 4 (FIG. 5B).

ssAAV2-smCBA-STC-1-FLAG Reduces Intraocular Pressure in a Sustained Manner

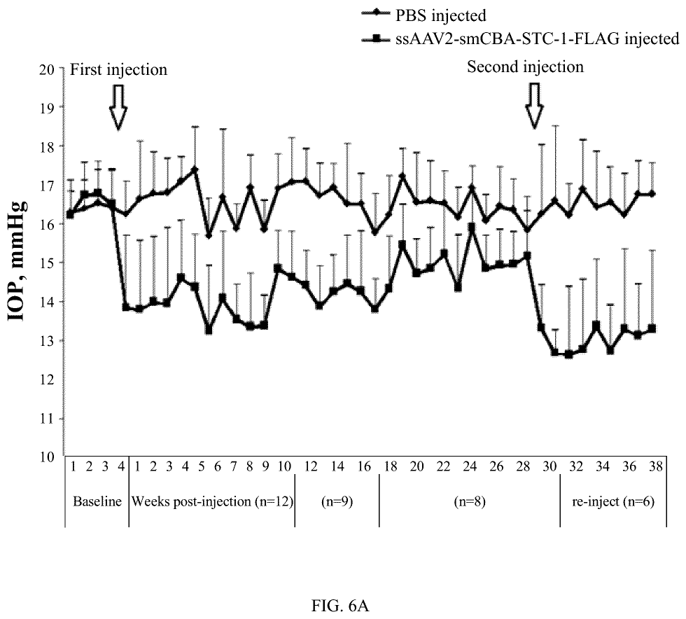

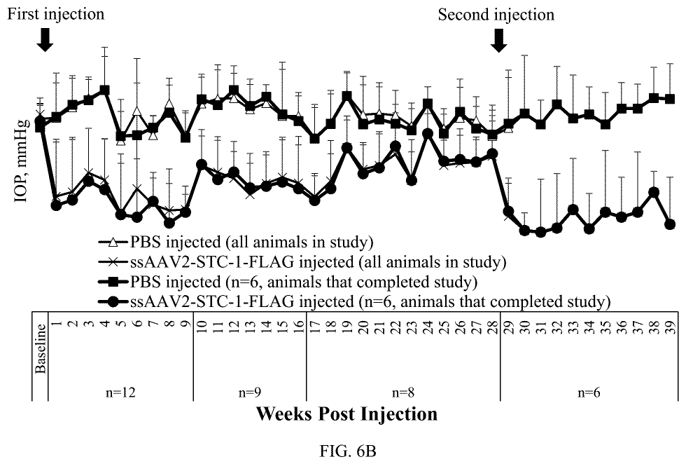

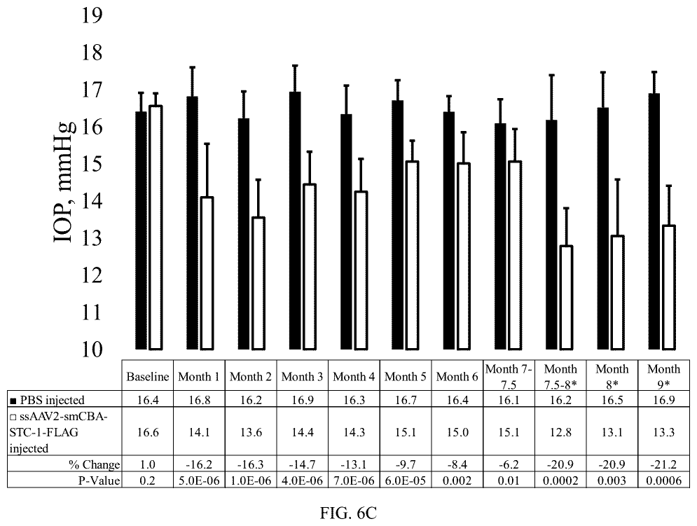

In order to determine whether STC-1 expression in the anterior chamber results in IOP reduction in a sustained manner, C57BL/6J mice with no difference in baseline IOP between fellow eyes (16.4±0.5 vs 16.6±0.4 mmHg, P=0.2, n=12, FIG. 6A-C), received a single intracameral injection of ssAAV2-smCBA-STC-1-FLAG (3×109 VGs) in one eye and an intracameral injection of PBS in the fellow eye. Averaged weekly IOP measurements showed IOP reduction in ssAAV2-smCBA-STC-1-FLAG injected eyes compared to PBS injected fellow eyes from week 1 through week 28 following the single intracameral injection (FIG. 6A). This experiment is an extended timeline of observation from the same initial experiment described in Example 1 (see, e.g., FIGS. 3, 4). For the first 4 months, IOP in the ssAAV2-smCBA-STC-1-FLAG injected eye was lower compared to the contralateral eye by 13.1-16.3% (>2.1 mmHg). In months 5 and 6, the IOP reduction was slightly less with percent change of 8.4-9.7% (>1.3 mmHg). Statistically, IOP reduction remained significant from month 1 with a 16.2% reduction (16.8±0.8 vs 14.1±1.4 mmHg, P<0.001, n=12, FIG. 6C) to month 7 with a 6.2% reduction (16.1±0.6 vs 15.1±0.9 mmHg, P=0.01, n=8, FIG. 6C). Though, the average IOP reduction at month 7 was less than 50% of the IOP reduction seen at 1 month, the difference was not statistically significant (14.1 vs 15.0 mmHg, P=0.2).

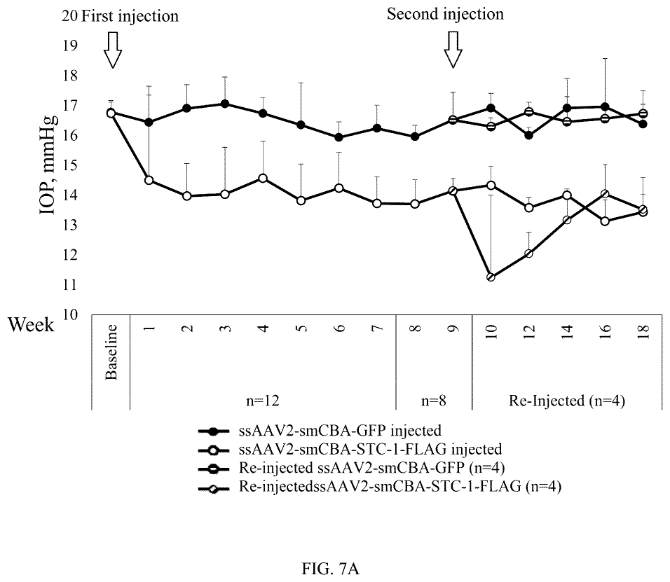

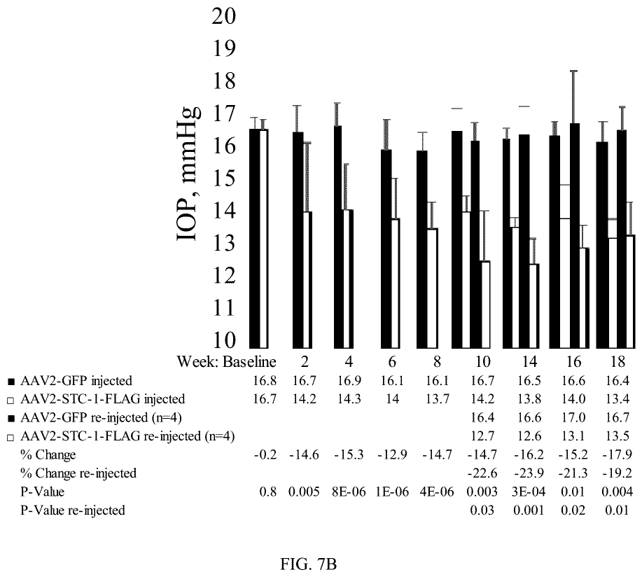

To determine whether a second injection would restore the IOP reduction observed within the first 4 months, eight animals were re-injected with a second dose of ssAAV2-smCBA-STC-1-FLAG or PBS in the fellow eye according to the original treatment protocol at 7.5 months (FIG. 6B). Two animals did not survive repeat anesthesia. One week following the second injection, IOP was 20.9% lower in eyes re-injected with ssAAV2-smCBA-STC-1-FLAG compared to fellow PBS re-injected control eyes (13.1±1.5 vs. 16.5±0.9 mmHg, P=0.0002, n=6, FIG. 6C) and remained lower for 2 additional months when the experiment was stopped to collect tissues for expression analysis (see below). Assessment of IOPs in animals at month 8 (second injection) compared to month 7 (single injection) showed that following the second injection of ssAAV2-smCBA-STC-1-FLAG, eyes had a significantly lower IOP (15.1±0.9 vs 12.8±1.0 mmHg, P<0.05). To determine whether the initial IOP lowering was superior following two injections compared to one injection, month 8 ssAAV2-smCBA-STC-1-FLAG twice-injected eyes were compared to once-injected ssAAV2-smCBA-STC-1-FLAG eyes at month 1 and no significant difference was seen (13.1±1.5 vs 14.1±1.4 mmHg, P=0.5). FIG. 7A contains a separate line graph showing the six re-injected animals are a representative sample of the original cohort.

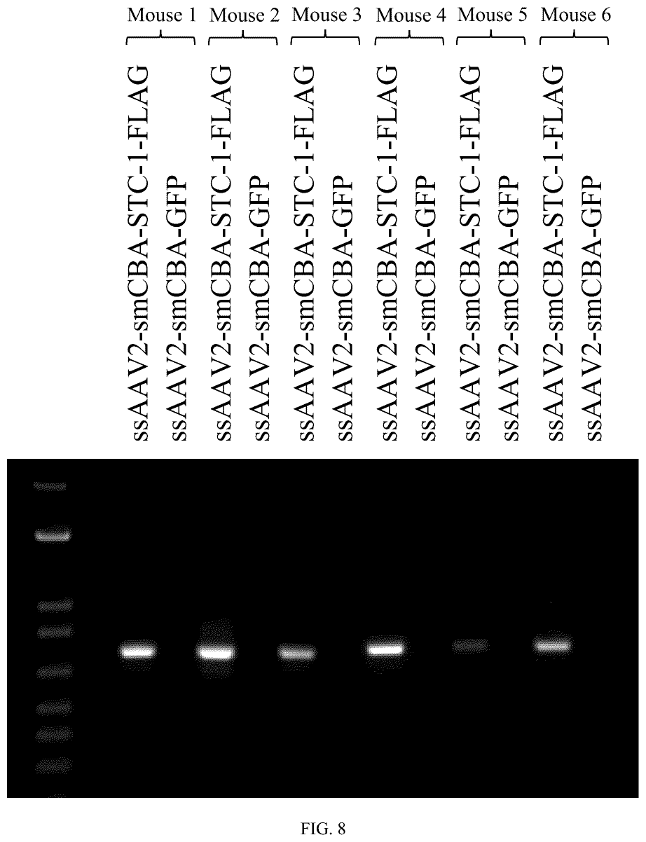

To assess STC-1-FLAG and GFP expression, ocular issues were collected from the remaining animals sacrificed at the completion of the experiment (week 38). Mice that received two intracameral injections of ssAAV2-smCBA-STC-1-FLAG showed RNA expression of the STC-1-FLAG transgene at the expected size of 728 base pairs (FIG. 8).

ssAAV2-smCBA-STC-1-FLAG Reduces Intraocular Pressure in a Sustained Manner Compared to ssAAV2-smCBA-GFP

In order to correlate tissue expression with repeat injections of ssAAV2-smCBA-STC-1-FLAG, C57BL/6J mice with no difference in baseline IOP between fellow eyes (16.6±0.4 vs 16.5±0.4 mmHg, P=0.4, n=12, FIG. 7A-7B) received an intracameral injection of ssAAV2-smCBA-STC-1-FLAG (3×109 VGs) in one eye and an intracameral injection of ssAAV2-smCBA-GFP (3×109 VGs) in the fellow eye. Eyes that received ssAAV2-smCBA-GFP showed no major change in IOP after injection throughout the course of the experiment (FIG. 7A-7B). In contrast, eyes that received ssAAV2-smCBA-STC-1-FLAG showed IOP reduction throughout the course of the experiment. At week 2 post injection, IOP was lowered an average of 14.6% in ssAAV2-smCBA-STC-1-FLAG injected eyes compared to fellow ssAAV2-smCBA-GFP injected eyes (14.2±2.1 vs 16.7±0.8 mmHg, n=12, P<0.001, FIG. 7B). Four animals were euthanized at week 8 for expression analysis. To address whether additional IOP-reduction could be obtained with a repeat injection, at week 9 half of the remaining 8 animals were randomized to a second injection (n=4). At week 10 of the experimental protocol (one week after the second injection), IOP was lowered 22.6% in ssAAV2-smCBA-STC-1-FLAG twice injected eyes compared to ssAAV2-smCBA-GFP twice injected eyes (12.7±1.6 vs. 16.4±1.6 mmHg, P<0.001, FIG. 7B). This trend continued until the completion of the experiment at week 18 when animals were euthanized for tissue isolation.

When comparing eyes that had received a single injection of ssAAV2-smCBA-STC-1-FLAG with eyes that received a second injection, both at week 10, the values were not statistically different (14.2±0.5 vs 12.7±1.6 mmHg, n=4, P=0.1, FIG. 7B). To determine whether IOP was lower one month after the second injection compared to 1 month after the first injection, week 14 twice injected ssAAV2-smCBA-STC-1-FLAG eyes were compared to week 4 once injected ssAAV2-smCBA-STC-1-FLAG eyes. Though there was a trend toward lower IOP (12.6±0.8 vs 14.3±1.4 mmHg, P=0.1, FIG. 7B), the results were not statistically significant.

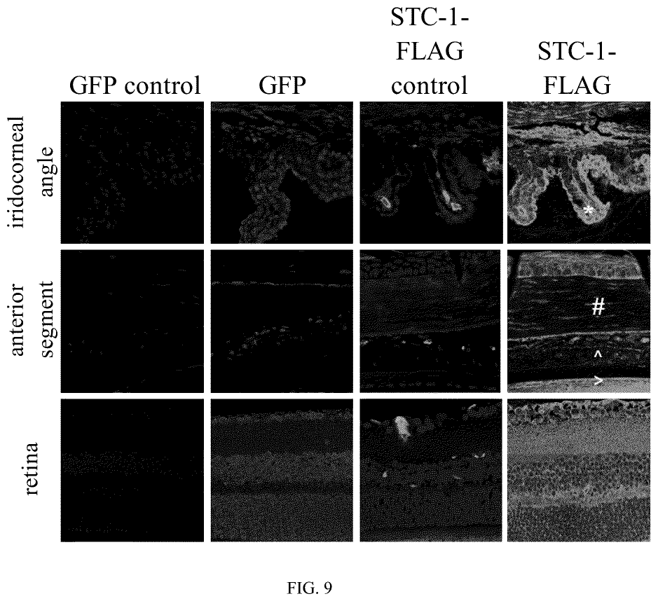

Eyes that received an intracameral injection of ssAAV2-smCBA-STC-1-FLAG showed diffuse STC-1-FLAG expression in the ciliary body, corneal endothelium, lens epithelium, iris, and retina (green fluorochrome) at week 8 (FIG. 9). Similar patterns of expression were seen at week 18 after a single intracameral injection and at week 18 after a second injection at week 10 (FIG. 10A-10B). Eyes that were injected with ssAAV2-smCBA-GFP (red fluorochrome) showed a similar pattern of expression to ssAAV2-smCBA-STC-1-FLAG in that expression could be detected in the ciliary body, corneal endothelium, lens epithelium, iris, and retina (FIG. 10A-10B).

Repeat Subconjunctival Injections Sustainably Lowers IOP

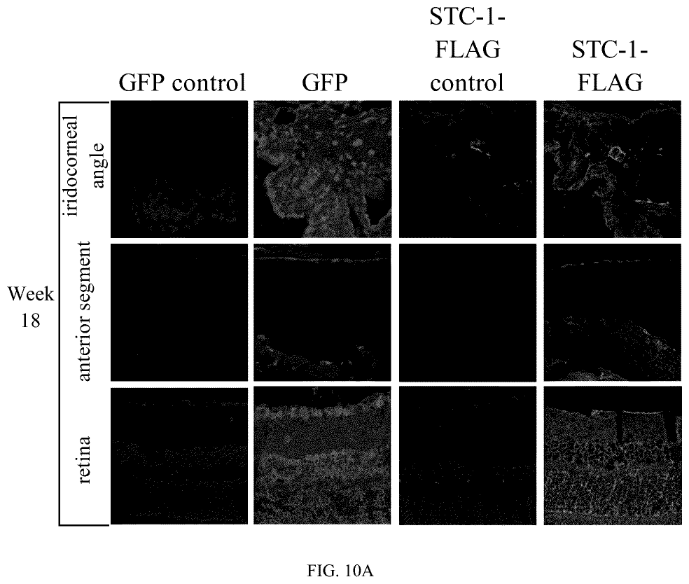

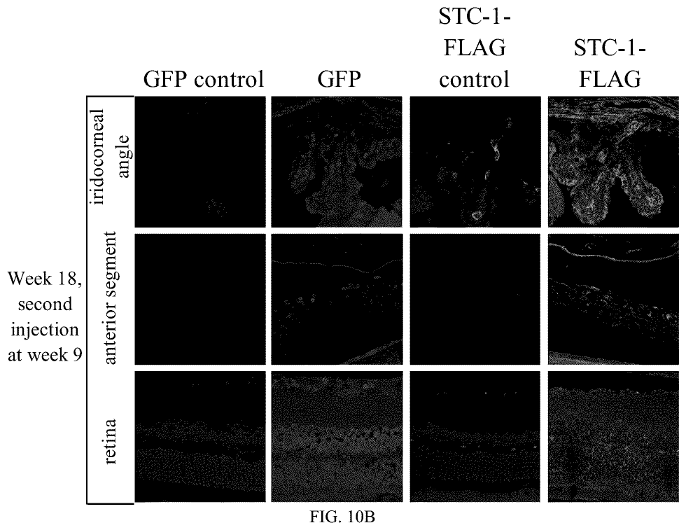

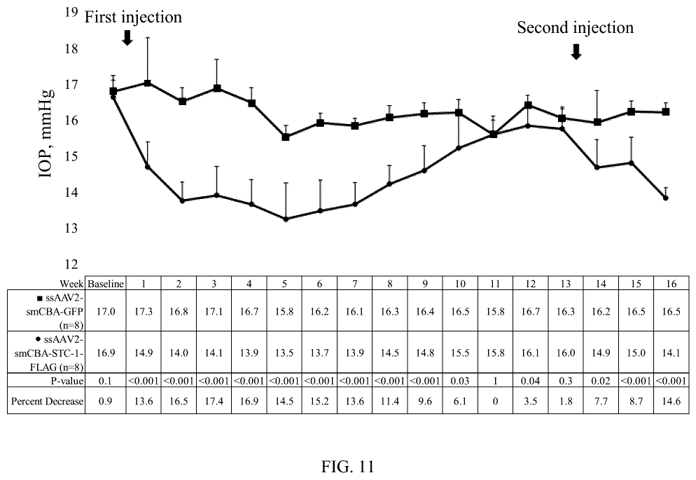

ssAAV2-smCBA-STC-1-FLAG (3×1012 VG/mL; 2 μL; 6×109 VGs) was injected in a sub-conjunctival manner (FIG. 2) after baseline IOP measurements. The same volume and titer of ssAAV2-smCBA-GFP was given in the fellow eye. Greater than 15% IOP reduction was seen in eyes injected with ssAAV2-smCBA-STC-1-FLAG compared to controls for the first month (FIG. 11). After this, the IOP-lowering effect began to wane and eventually lost statistical significance. At the end of experimental week 13, when IOP had returned to baseline, animals were re-injected with the same volume and titer of the initial AAV construct. IOP reduction 2 weeks after second injection was re-established (experiment still in progress) (FIG. 11).

ssAAV2-smCBA-STC-1-FLAG Reduces Intraocular Pressure by Increasing Outflow Facility

In order to determine the mechanism of action of IOP lowering by ssAAV2-smCBA-STC-1-FLAG, aqueous outflow measurements were performed. Three-month-old C57BL/6J mice (n=14) were randomized to treatment with a single injection of intracameral ssAAV2-smCBA-STC-1-FLAG (n=5), a single injection of intracameral ssAAV2-smCBA-GFP (n=5), or a no treatment control (n=4). In addition, a separate cohort of age-matched C57BL/6J mice (n=9) were randomized to treatment with daily topical administration of LFA (n=5) or STC-1 (n=4). At maximal IOP reduction (6 weeks post injection for intracameral injections and treatment day 5 for topical treatments), aqueous humor dynamic studies by perfusion were performed. Eyes injected with ssAAV2-smCBA-STC-1-FLAG showed a statistically significant decrease in IOP as well as an increase in outflow facility when compared to untreated controls or ssAAV2-smCBA-GFP injected eyes (Table 8). Likewise, topically administered STC-1 also showed IOP reduction and an increase in outflow facility. This was similar to results obtained for topical LFA which showed a decrease in IOP and an increase in outflow facility suggesting a similar mechanism of action to STC-1. No change in uveoscleral outflow, episcleral venous pressure or aqueous inflow was seen in any of the treatment groups (Table 8).

TABLE 8
ssAAV2-smCBA-STC-1-FLAG reduces IOP by increasing outflow facility

| | | ssAAV2- | | | |
|---|---|---|---|---|---|
| | ssAAV2- | smCBA- | | Topical | Topical |
| | smCBA- | STC-1- | Untreated | LFA | STC-1 |
| | GFP (n = 5) | FLAG (n = 5) | (n = 4) | (n = 5) | (n = 4) |
| Outflow facility, | 0.016 ± 0.005 | 0.038 ± 0.008 | 0.025 ± 0.006 | 0.046 ± 0.02 | 0.048 ± 0.02 |
| μL/min/mmHg | | | | | |
| ssAAV2-smCBA- | | | | | |
| GFP | | | | | |
| ssAAV2-smCBA- | P = 0.001 | | | | |
| STC-1-FLAG | | | | | |
| Untreated | P = 0.05 | P = 0.03 | | | |
| Topical LFA | P = 0.008 | P = 0.4 | P = 0.06 | | |
| Topical STC-1 | P = 0.003 | P = 0.3 | P = 0.03 | P = 0.9 | |
| Uveoscleral | 0.078 ± 0.03 | 0.044 ± 0.03 | 0.04 ± 0.03 | 0.046 ± 0.02 | 0.043 ± 0.03 |
| outflow, μL/min | | | | | |
| Episcleral venous | 12.2 ± 1.6 | 11.0 ± 0.7 | 12.5 ± 2.7 | 10.0 ± 2.4 | 10.5 ± 1.6 |
| pressure, mmHg | | | | | |
| Measured IOP, | 16.0 ± 1.2 | 13.4 ± 0.5 | 15.9 ± 0.5 | 13.2 ± 0.9 | 13.2 ± 0.6 |
| mmHg | | | | | |
| Calculated IOP, | 15.7 ± 1.3 | 13.8 ± 1.4 | 14.8 ± 1.7 | 14.0 ± 1.9 | 12.1 ± 0.9 |
| mmHg | | | | | |

### Example 3: Virally Expressed Stanniocalcin-1 is Anti-Inflammatory and Neuroprotective

This example demonstrates the anti-inflammatory and neuroprotective properties of STC-1.

### Methods

AAV2 (Triple Y-F) (Petrs-Silva et al. Mol Ther. 19(2):293-301(2011)) was used to deliver the human STC-1 coding region (SEQ ID NO:3) with the signal peptide and a C terminal FLAG tag using the chicken β-actin (CBA) ubiquitous promotor (FIG. 1A). Wild-type mice were administered a single intracameral injection to the anterior chamber of the eye (FIG. 2) 1 μL of ssAAV2-smCBA-STC-1-FLAG (3.28E+12 VG/mL), and the same volume of PBS was injected in the fellow eye using a 32 g Hamilton needle and syringe. IOP was measured twice weekly with a handheld rebound tonometer following injection.

DBA/2J mice were injected by intravitreal injection at 2 months of age with ssAAV2-smCBA-STC-1-FLAG in one eye and ssAAV2-smCBA-GFP in the fellow eye. IOP was recorded weekly for 9 months.

Experimental autoimmune uveitis (EAU) was induced in the C57B/6 mouse with a protocol described elsewhere (see, e.g., Avichezer et al., Invest. Ophthalmol. Vis. Sci., 41(1):127-31 (2004)). Briefly, human interphotoreceptor retinoid binding protein (IRBP) in complete Freund's adjuvant was injected subcutaneously and pertussis toxin was injected intraperitoneally as an additional immune stimulus.

### Results

STC-1 is Neuroprotective

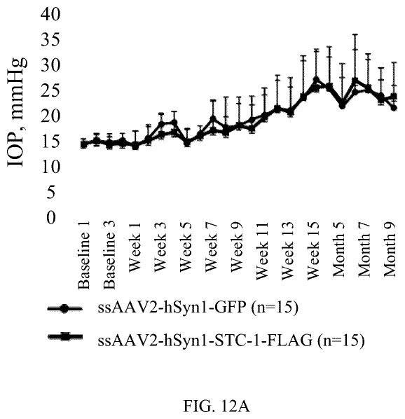

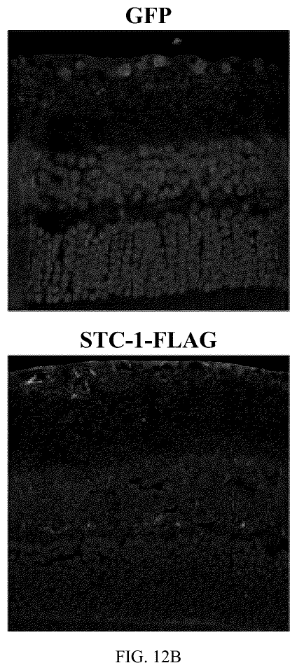

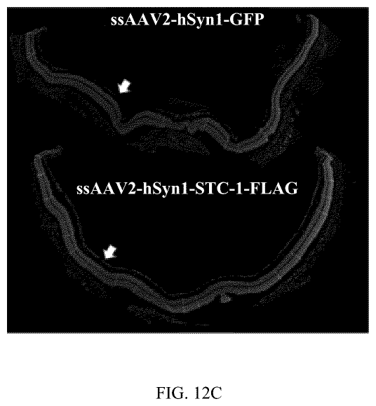

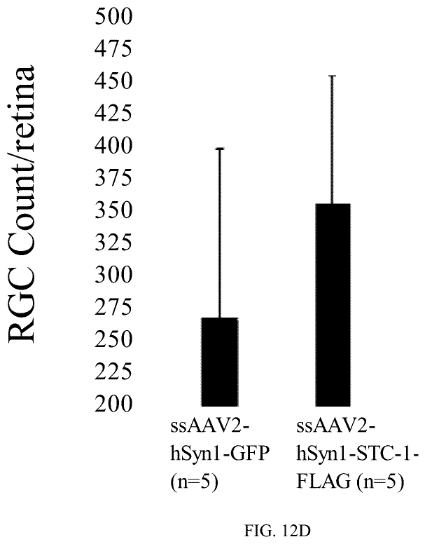

In 2-month-old DBA/2J mice administered intravitreal injections of ssAAV2-hSyn1-STC-1-FLAG in one eye and ssAAV2-hSyn1-GFP in the fellow eye, no difference in IOP was observed throughout the experimental period (FIG. 12A). At the end of the experimental period whole eyes were collected, stained, and retinal ganglion cell counts were performed. Expression of GFP was observed in ssAAV2-hSyn1-GFP-injected eyes, and expression of STC-1-FLAG was observed in ssAAV2-hSyn1-STC-1-FLAG-injected eyes at the end of the experimental period (FIG. 12B). Retinas in eyes injected with ssAAV2-hSyn1-GFP show the age-expected sparse retinal ganglion cell (RGC) layer with regions of complete dropout while eyes injected with ssAAV2-hSyn1-STC-1-FLAG show preservation of the RGC layer (FIG. 12C). Despite the initial cohort comprising a limited sample size, eyes injected with ssAAV2-hSyn1-STC-1-FLAG had 50% more retinal ganglion cells (RGCs) than fellow s sAAV2-hSyn1-GFP injected control eyes after 9 months (FIG. 12D). RGC loss is the hallmark of optic neuropathies including glaucoma (Smith et al., Eye, 31:209-17(2017)). The data suggest that intravitreal administered ssAAV2-hSyn1-STC-1-FLAG provides neuroprotection independent of IOP reduction.

STC-1 is Anti-Inflammatory

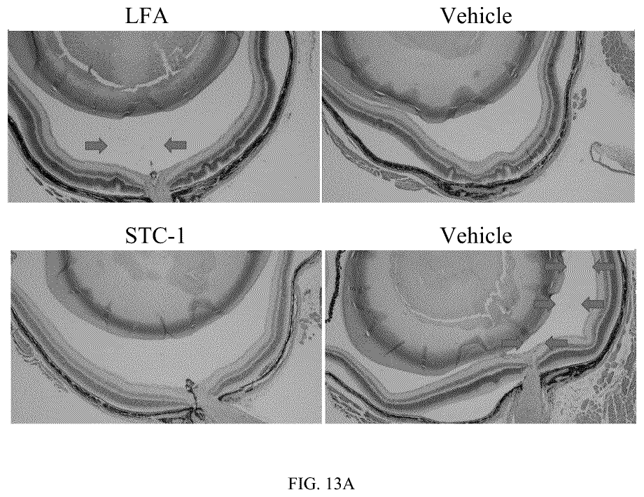

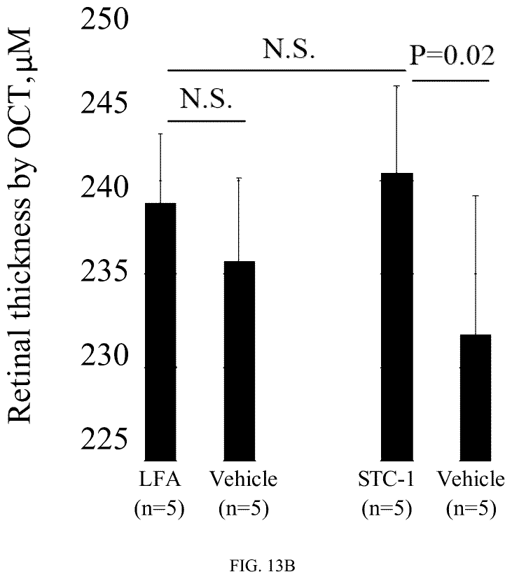

In C57B/6 mice induced with experimental autoimmune uveitis (EAU) a trend toward fewer inflammatory cells was observed in the vitreous in STC-1 treated mice was observed (n=5; FIG. 13A). Retinal thickness was preserved in STC-1 treated mice compared to vehicle control (n=5, p=0.02; FIG. 13B). Retinal thinning by OCT is a marker of retinal damage in EAU.

Taken together, these results demonstrate that STC-1 can reduce intraocular inflammation, and that sustained expression of STC-1 by an AAV can provide neuroprotection (e.g., neuroprotection independent of IOP reduction).

### Example 4: ssAAV-STC-1 Reduces IOP Independent of the FP Receptor

This Example demonstrates that IOP-lowering properties of ssAAV2-smCBA-STC-1-FLAG are not dependent on the FP receptor.

### Methods

FP receptor knockout mice were generated using CRISPR/Cas9 (Roddy et al., PLoS One A Schier stop cassette was introduced within exon 2 of the Ptfr gene, producing a non-functional truncated FP receptor protein product.

Following 4 consecutive days of baseline IOP measurements, 3-month-old FP receptor knockout mice (n=7) received a single intracameral injection of 1 μL ssAAV2-smCBA-STC-1-FLAG (3E+12 VG/mL) in one eye and the same volume and copy number of ssAAV2-smCBA-GFP was injected into the fellow eye. IOP was measured twice weekly starting day four post-injection with a handheld rebound tonometer.

### Results

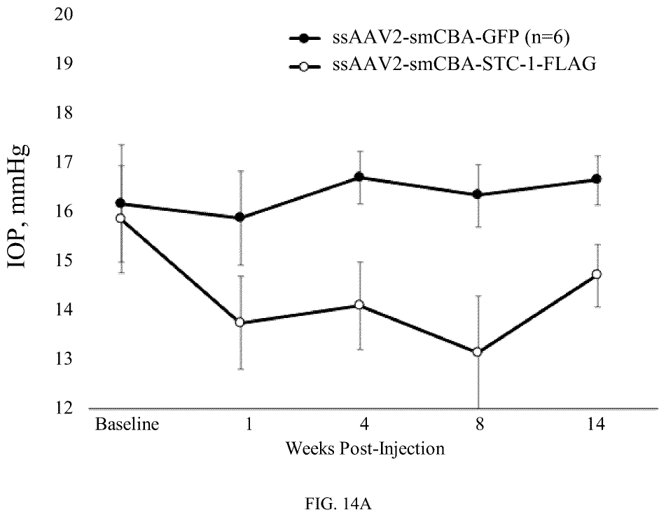

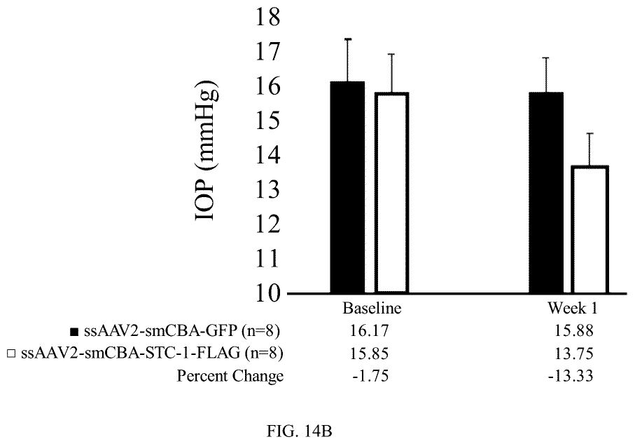

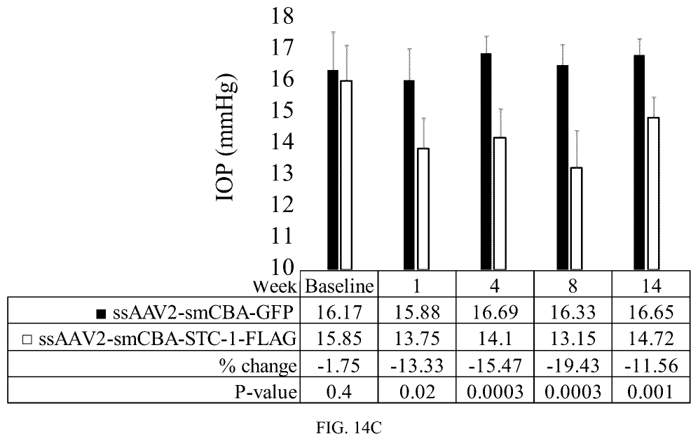

To determine whether sustained IOP reduction following ssAAV2-smCBA-STC-1-FLAG injection was regulated by the FP receptor, intracameral injections of ssAAV2-smCBA-STC-1-FLAG were administered in one eye and ssAAV2-smCBA-GFP in the fellow eye of FP receptor knockout mice previously generated (Roddy et al., Mol Ther. 20:788-97(2012)). There was no difference in baseline IOP measurements between fellow eyes (16.3±0.8 vs 16.3±0.9 mmHg, n=6, P=0.8, FIG. 14A-B). Compared to fellow control eyes, IOP was reduced in FP receptor knockout mice injected with ssAAV2-smCBA-STC-1-FLAG by 15.5% at month 1 (16.3±1.1 vs 14.0±0.7 mmHg, n=6, P<0.001, FIG. 14C) and by 19.4% (16.2±0.4 vs 13.5±1.1 mmHg, n=6 P<0.001, FIG. 14C) at month 2. FIG. 14C is an extended timeline of observation from the same initial experiment illustrated in FIG. 14B. This suggests that sustained IOP reduction with STC-1-FLAG expression is not dependent on the FP receptor.

These results demonstrate that AAV-STC-1 can be used to provide sustained IOP reduction downstream of the first line topical glaucoma medication latanoprost and downstream of the FP receptor. AAV-STC-1 may be a therapeutic that can revolutionize glaucoma patient care and compliance rates, as the interval dosage regimen, anti-inflammatory, neuroprotective and IOP-lowering benefits of STC-1 are demonstrable improvements over current day standard of care topical prostaglandin treatments.

TABLE 9
Comparison between prostaglandin and
AAV-STC-1 treatment modalities.

| | Topical | |
|---|---|---|
| | prostaglandin | AAV- |
| | (standard of care) | STC-1 |
| Daily dosing | X | |
| Activation of pro-inflammatory | X | |
| pathways | | |
| Ocular surface side effects | X | |
| Avoidance of the FP receptor | | X |
| Minimization of IOP fluctuation | | X |
| Monthly/quarterly/yearly dosing? | | X |
| Minimization of involuntary | | X |
| compliance problems | | |
| Minimization of voluntary | | X |
| compliance problems | | |
| Neuroprotection, | | X |
| anti-inflammatory/anti-oxidant | | |
| effects | | |

### Example 5: Risk Factors for Radiation Papillopathy Following Plaque Radiotherapy for Uveal Melanoma

Some cases of radiation papillopathy displayed optic disc pallor as the predominant feature, while others developed concomitant neuroretinal rim thinning (NRT). This Example demonstrates that higher radiation dose to optic disc and higher baseline IOP are risk factors for radiation papillopathy.

### Methods

Retrospective chart review was performed on a subset of patients enrolled in the Prospective Ocular Tumor Study (POTS). Included patients were diagnosed with uveal melanoma involving the choroid and/or ciliary body and treated with plaque radiotherapy. Patients were required to have a minimum of three years follow-up after plaque and at least two fundus photographs containing a clear, unobstructed view of the optic disc, one prior to plaque and one ≥3 years after treatment. Patients with isolated iris melanoma, <3 years follow-up, or lack of clear photographs of the optic disc were excluded.

All patients underwent a complete eye examination by an ocular oncologist (LAD, TWO) with color fundus drawing, fundus photography, autofluorescence, A and B scan ultrasonography, fluorescein angiography (FA), and optical coherence tomography (OCT) as indicated. Plaque radiotherapy was performed under anesthesia using I-125 Collaborative Ocular Melanoma Study (COMS) plaques with tumor localization using transillumination and/or indirect ophthalmoscopy. Intraoperative ultrasonography was used to confirm proper plaque placement. Planned treatment duration was approximately 94 hours. Records were reviewed for patient demographics, clinical tumor features, treatment parameters, and outcomes, with attention to IOP and optic disc features.

Radiation papillopathy was defined as first appearance of optic disc edema or diffuse optic disc pallor. Sectoral disc pallor associated with retinal vein occlusion or TTT was not counted as radiation papillopathy. Disc color over time was compared with baseline optic disc color of the treated eye and disc color of the fellow eye at each matching time point. Attention was taken not to overestimate optic disc pallor if the treated eye was rendered pseudophakic by considering overall change in fundus color from baseline. Detailed optic disc data were collected from photographic review before treatment and after treatment when available at 6, 12, 24, 36, 48, 60, and 120 months, including IOP, cup to disc ratio (C/D), inferior and superior optic disc rim (expressed as percentage of total disc vertical height), Drance hemorrhage, disc edema, diffuse pallor, neovascularization of the disc (NVD), and treatment with IOP-lowering therapy. All images were graded by a single observer (LAD) who was masked to all clinical data. Optic disc rim was measured using calipers at the 12:00 and 6:00 positions. Any hesitations by the grader (LAD) were addressed by consensus review with a glaucoma specialist (GWR).

Radiation dosages to the tumor, fovea, and optic disc were calculated using EyeDose. The program utilizes Monte Carlo methods to calculate three-dimensional heterogeneity corrected dose distributions for 1-125 COMS plaques and has been validated against published Monte Carlo results for eight different tumor positions.

Statistical analysis was performed using SPSS Statistics Software Version 22 (IBM, Armonk, New York). Demographics, clinical features, treatment features, and outcomes were compared for eyes with no radiation papillopathy, papillopathy with pallor only, and papillopathy with pallor plus NRT. Categorical variables were compared using Fisher's exact test, and continuous variables were compared using the Kruskal-Wallis H test. A subanalysis was performed to compare eyes affected by radiation papillopathy manifesting as pallor only versus pallor plus NRT. Categorical variables were compared using Fisher's exact test, and continuous variables were compared using the Mann-Whitney U test. Logistic regression analysis was used to determine risk factors for radiation papillopathy by univariate and multivariate analysis using the stepwise method. The Benjamini-Hochberg procedure was used to control the false discovery rate for multiple comparisons using a q-value of 0.10. A P-value <0.05 was considered statistically significant.

### Results

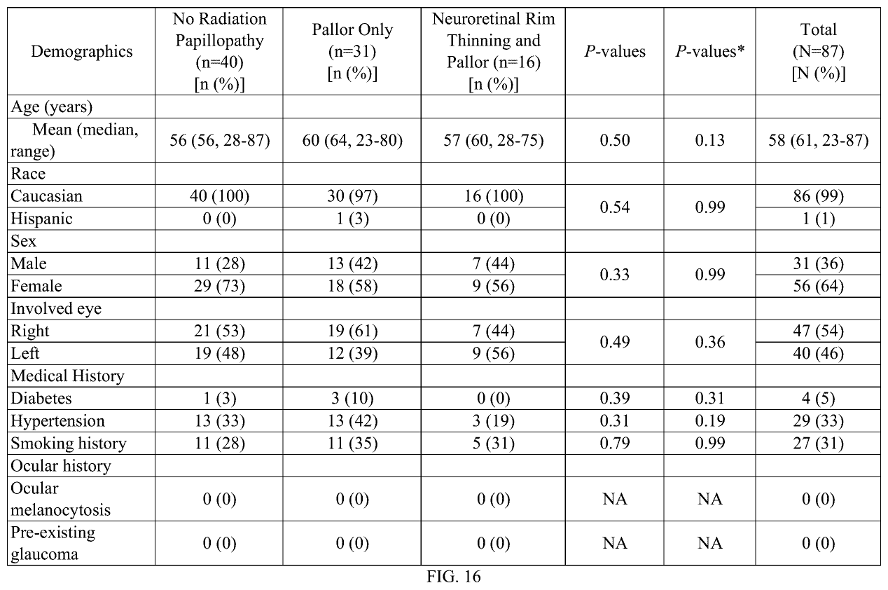

There were 87 eyes of 87 patients included in the study. Of these, 40 (46%) developed no radiation papillopathy and 47 (54%) developed radiation papillopathy, with pallor only in 31 (36%) or pallor and NRT in 16 (18%) (FIG. 15). Patient demographics are described in FIG. 16, and a comparison (no papillopathy vs. pallor vs. pallor and NRT) revealed no differences between groups.

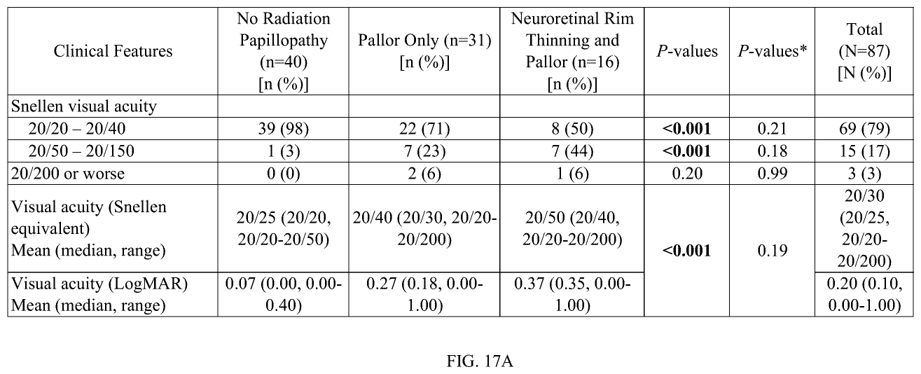

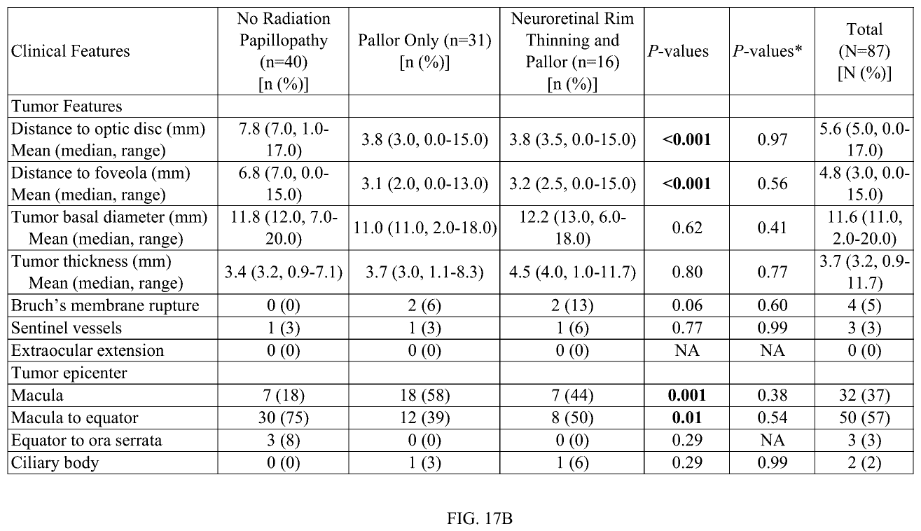

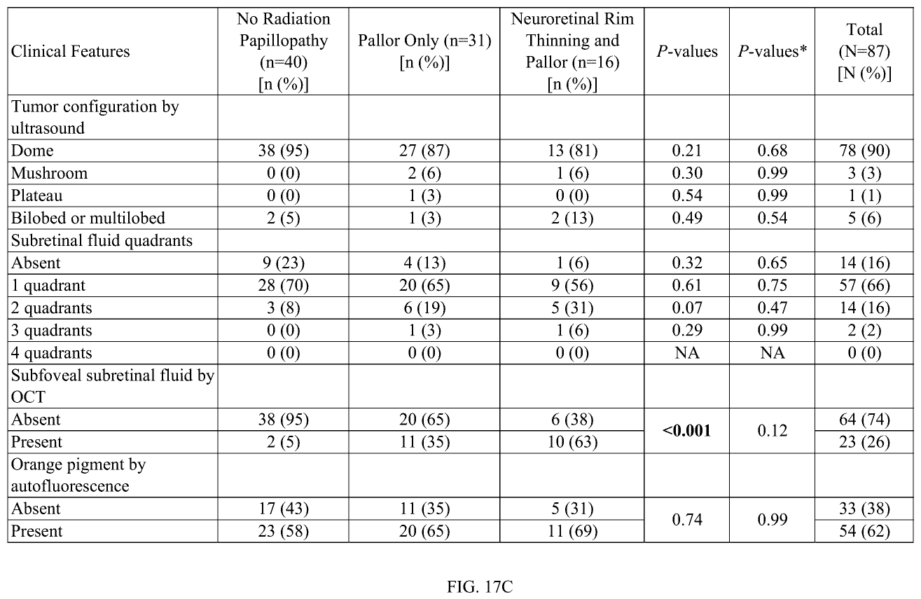

Clinical features are detailed in FIG. 17A-C. A comparison (no papillopathy vs. pallor vs. pallor and NRT) revealed eyes with papillopathy of any type presented with worse LogMAR visual acuity (P<0.001), shorter tumor distance to optic disc (P<0.001) and foveola (P<0.001), and more frequent subfoveal fluid (P<0.001).

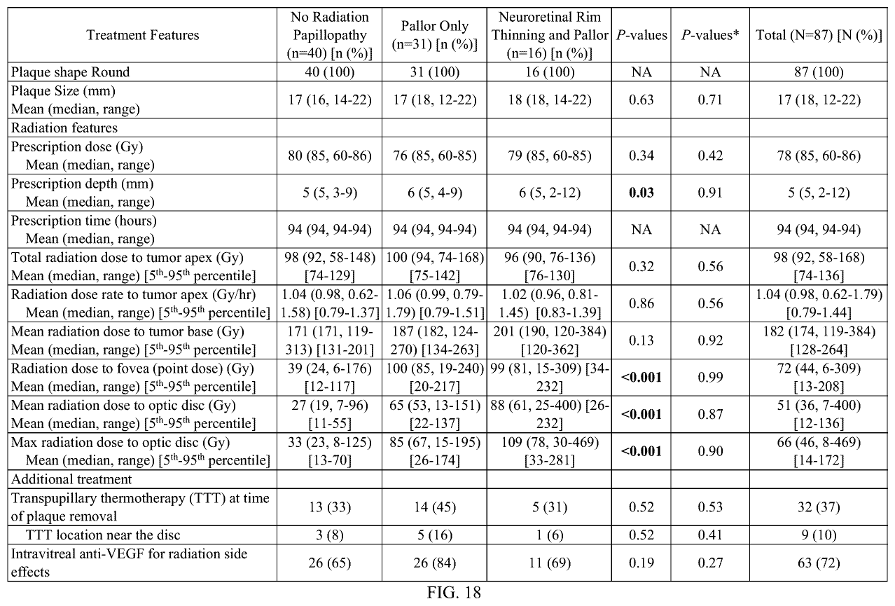

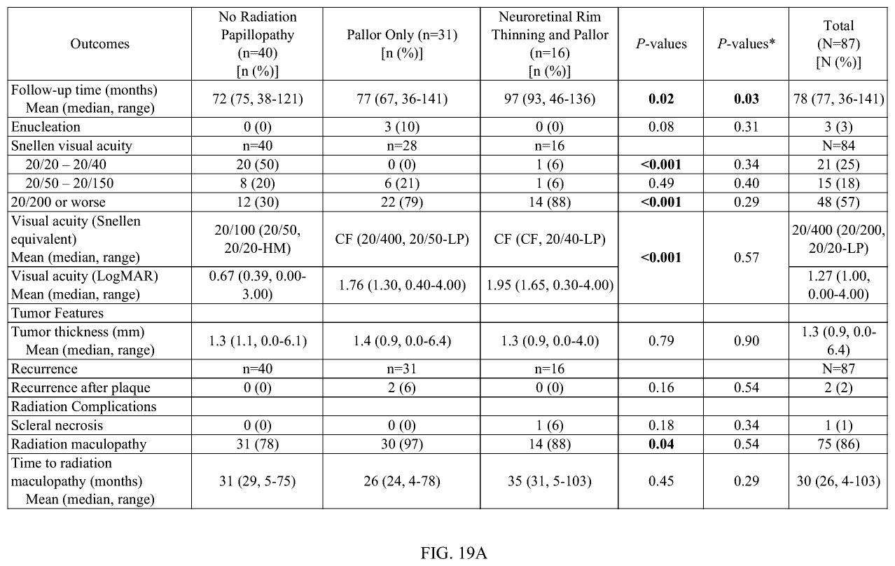

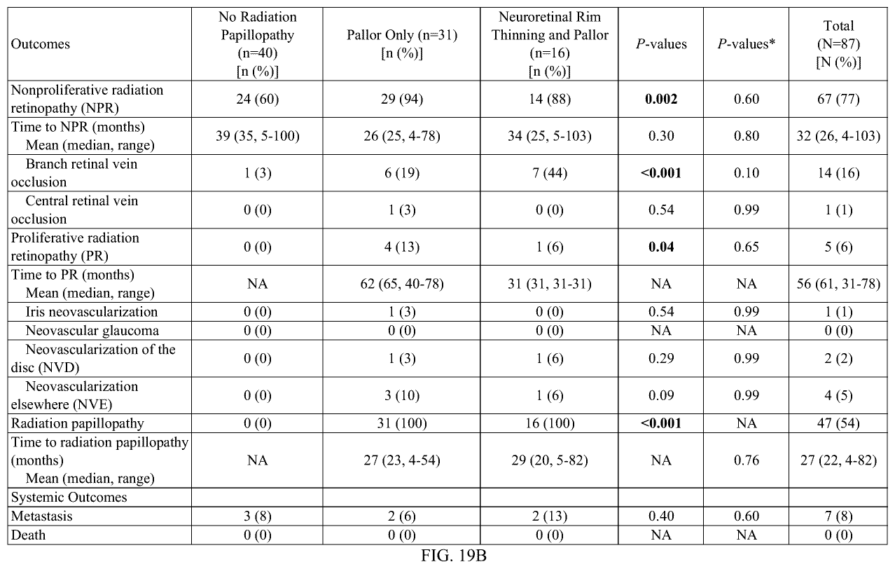

Treatment features are described in FIG. 18. A comparison (no papillopathy vs. pallor vs. pallor and NRT) revealed eyes with papillopathy of any type were treated with greater prescription depth (P=0.03), greater radiation dose to fovea (point dose) (P<0.001), greater mean radiation dose to optic disc (P<0.001), and greater maximum radiation dose to optic disc (P<0.001). No specific treatment was recommended for radiation papillopathy, although 72% of all study eyes received intravitreal anti-VEGF at some point for radiation retinopathy and/or maculopathy.

Clinical outcomes are described in FIG. 19A-B. A comparison (no papillopathy vs. pallor vs. pallor and NRT) revealed eyes with both pallor and NRT had longer follow-up time (P=0.02). Eyes with radiation papillopathy of any type had worse final LogMAR visual acuity (P<0.001), greater frequency of radiation maculopathy (P=0.04), nonproliferative radiation retinopathy (P=0.002), branch retinal vein occlusion (P<0.001), and proliferative radiation retinopathy (P<0.001). There were no cases of neovascular glaucoma. There was no difference in time to radiation papillopathy by papillopathy subtype (pallor only vs. pallor and NRT) (27 vs. 29 months, P=0.76).

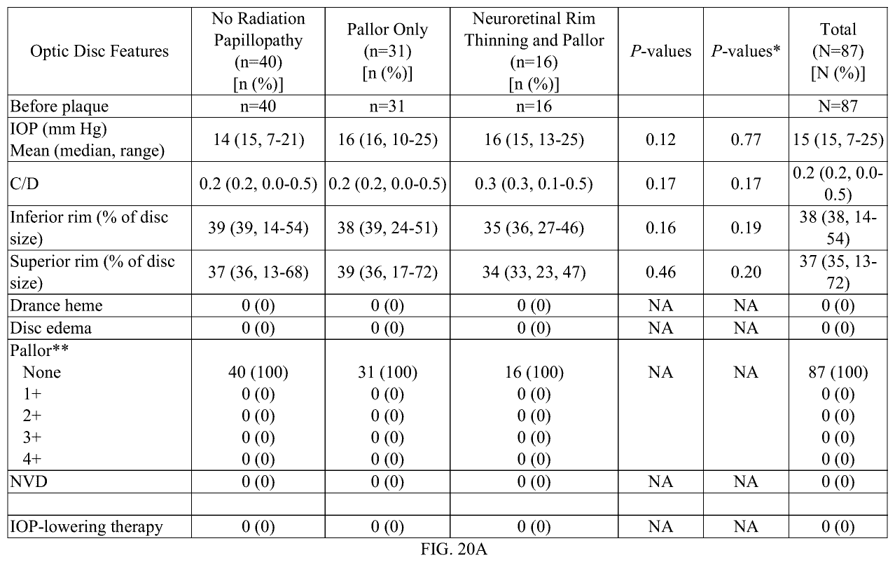

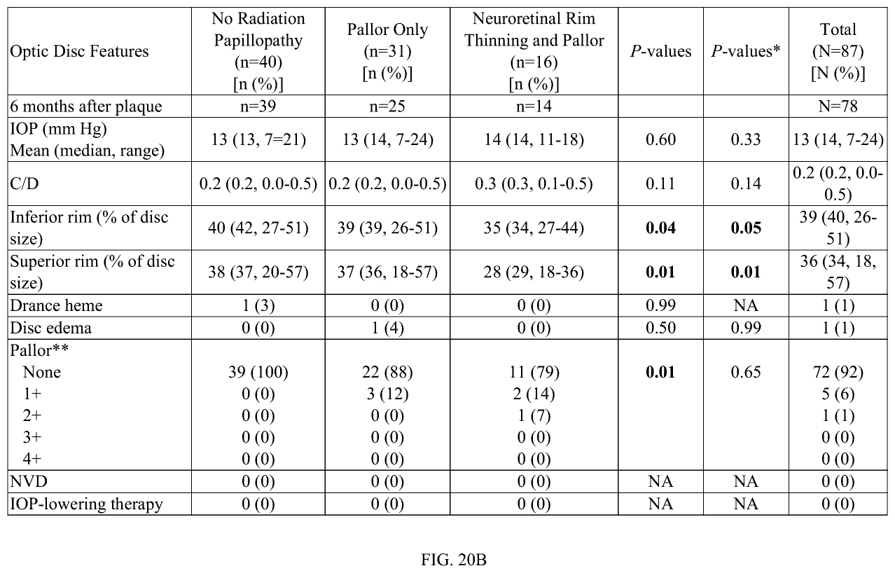

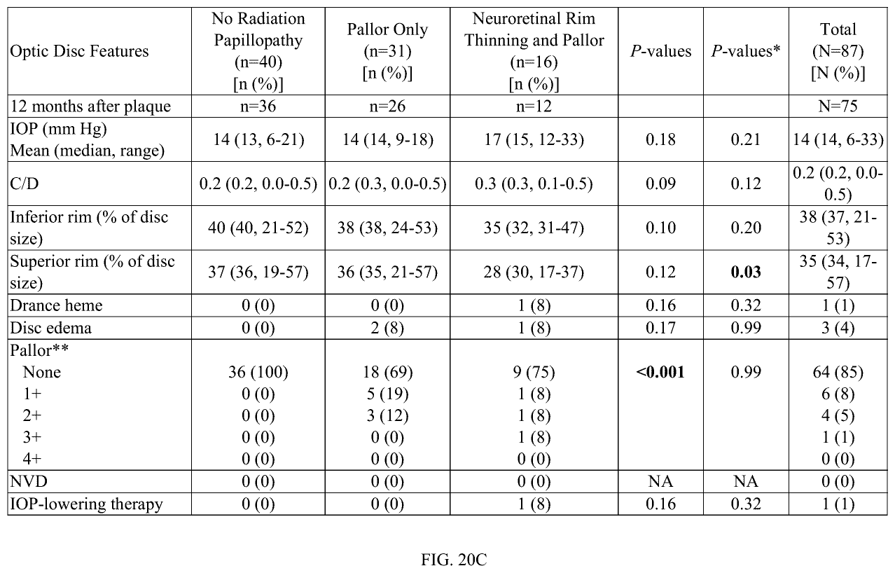

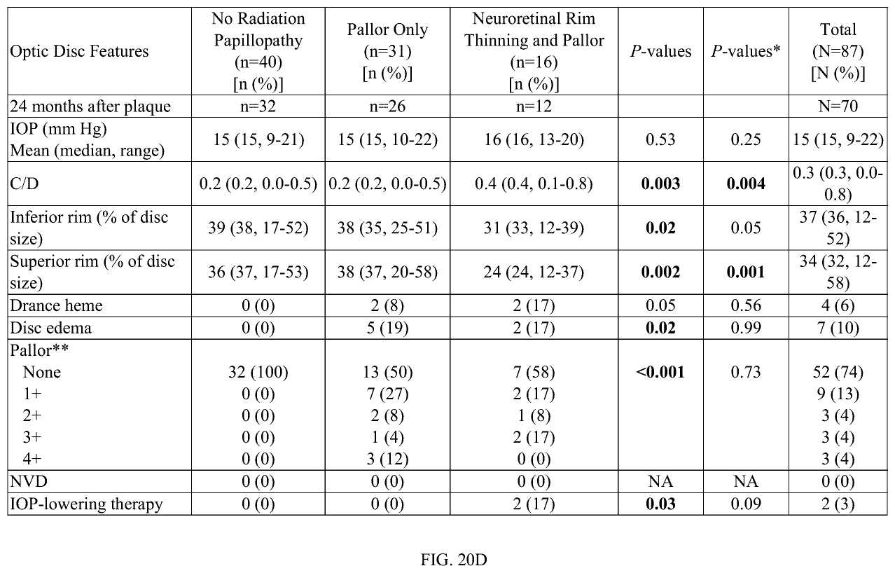

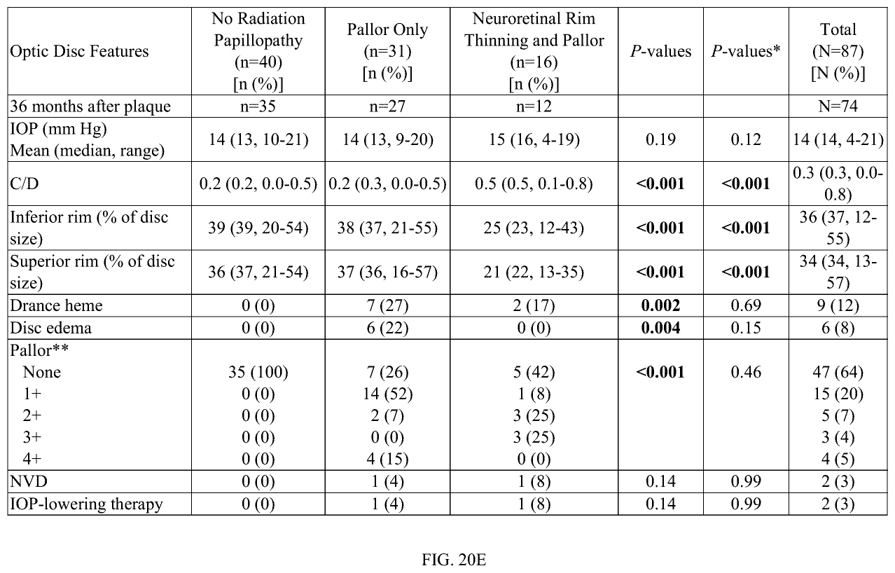

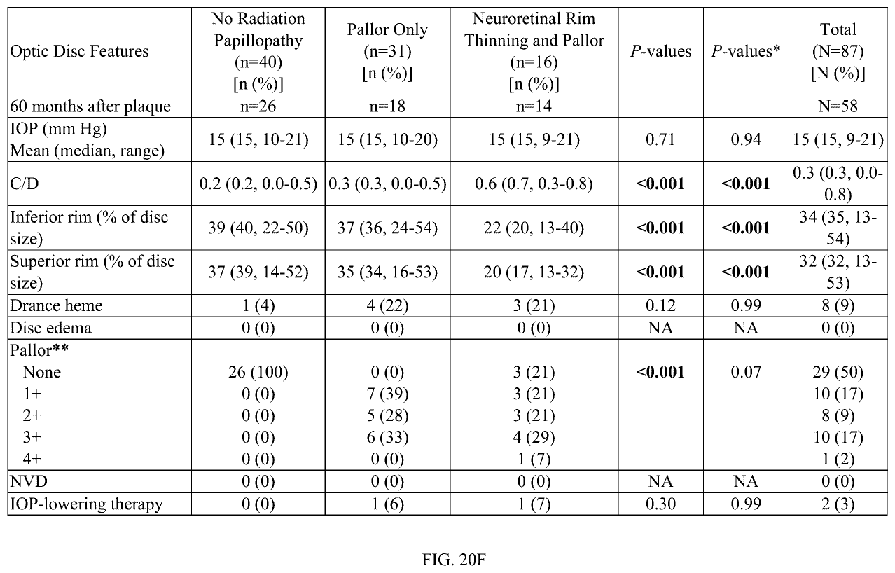

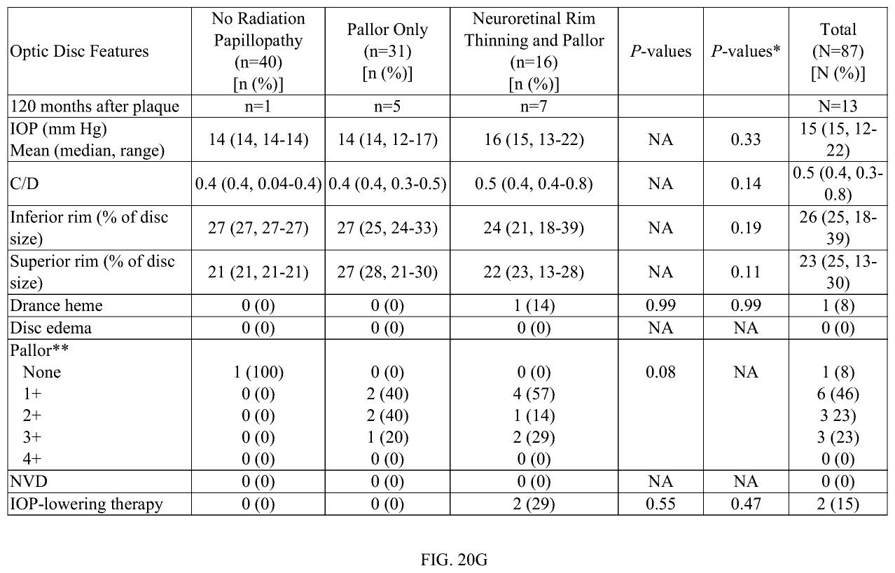

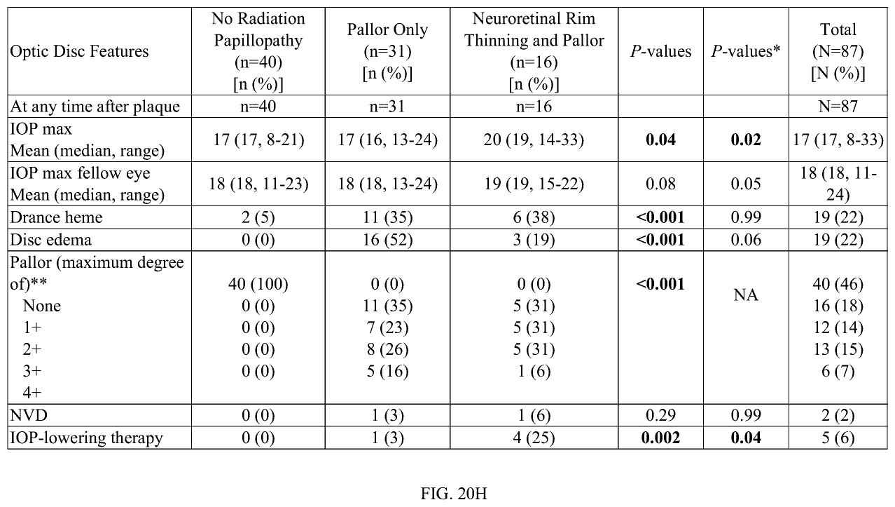

Detailed optic disc features over time are listed in FIG. 20A-20H. No eyes had baseline glaucoma, Drance hemorrhage, disc edema, pallor, or NVD, and no eyes were being treated with IOP-lowering therapy. Eyes with any type of radiation papillopathy had greater frequency of Drance hemorrhage (P<0.001). Eyes with pallor only had the greatest frequency of disc edema (P<0.001), and eyes with pallor and NRT had the greatest maximum IOP after plaque (P=0.04) and most frequent requirement for topical IOP-lowering therapy (P=0.002). One eye in the pallor and NRT group had a single time point spike in IOP to 33 mmHg at 12 months after plaque, which responded to topical dorzolamide hydrochloride-timolol maleate. There were no other instances of IOP higher than 25 mmHg. No fellow eyes developed ocular hypertension, NRT, glaucoma, or other optic neuropathies during the study period. On subanalysis comparing radiation papillopathy eyes with pallor alone versus pallor and NRT, eyes with both pallor and NRT had greater maximum IOP after plaque (P=0.02) and more frequent requirement for topical IOP-lowering therapy (P=0.04), but there was no difference in the overall frequency of Drance hemorrhage or optic disc edema.

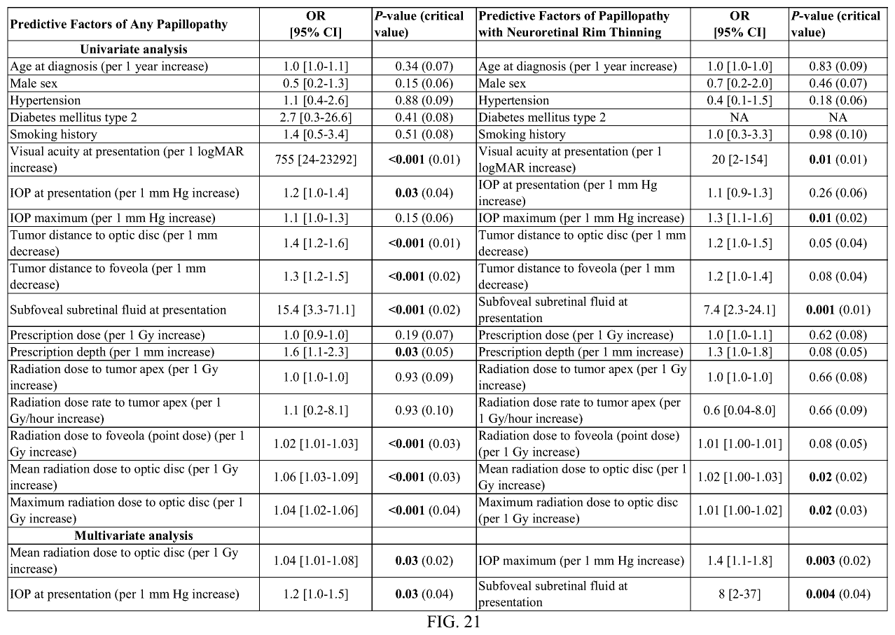

Factors associated with radiation papillopathy on univariate and multivariate logistic regression analysis are listed in FIG. 21. A primary analysis was performed for patients with any type of radiation papillopathy (pallor with or without NRT), and a model was constructed for multivariate analysis using objective factors that were significant on univariate analysis, including IOP at presentation, subfoveal subretinal fluid, radiation prescription depth, radiation dose to fovea, and mean radiation dose to optic disc. Significant predictors of all type papillopathy on multivariate analysis were greater mean radiation dose to optic disc (P=0.03) and higher baseline IOP (P=0.03). A subanalysis was performed to examine risk factors specific to development of concomitant NRT and optic disc pallor. A model was constructed for multivariate analysis using objective factors that were significant on univariate analysis, including higher maximum IOP after plaque, subfoveal subretinal fluid at presentation, and mean radiation dose to optic disc. Significant predictors of papillopathy with NRT on multivariate analysis were higher maximum IOP (P=0.003) and subfoveal subretinal fluid (P=0.004). All significant variables remained significant after controlling the false discovery rate.

### Example 6: Stanniocalcin-1 Reduces Intraocular Pressure in Two Models of Ocular Hypertension

With the goal of being able to maintain the IOP-lowering properties of latanoprost, avoid the side-effects, and offer a novel therapy to PGF2α non-responders, Stanniocalcin-1 (STC-1) was identified as a downstream effector molecule in latanoprost-mediated IOP reduction (Roddy et al., Invest Ophthalmol Vis Sci. 58:2715-24(2017)). STC-1 was identified as an independent ocular hypotensive agent as it lowered pressure in the human anterior segment perfusion culture model and in wild-type C57BL/6J mice. Though the point of signaling overlap and divergence has not yet been defined, current data suggests that latanoprost induces expression of STC-1 downstream of the FP receptor in the pathway responsible for IOP reduction.

This Example demonstrates that administration of STC-1 reduces intraocular pressure in two distinct rodent models.

Methods

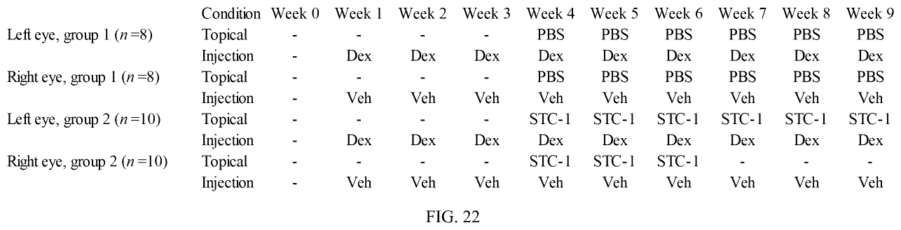

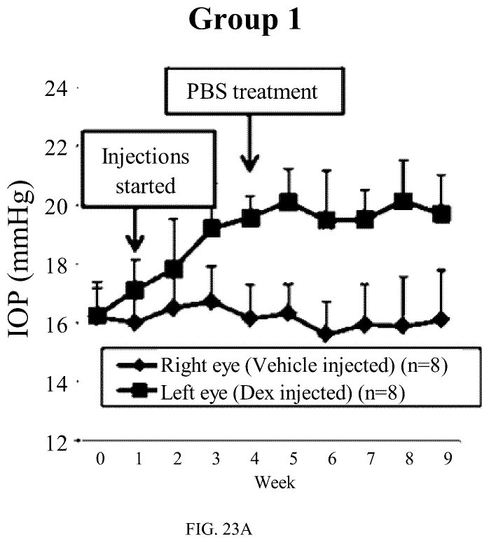

All studies were approved by the Mayo Clinic (Rochester, MN) IACUC and adhered to ARVO guidelines. To develop the steroid-induced ocular hypertension model, IOP was measured in both eyes of wild-type C57BL/6J mice (n=7, 6-8 months old) twice daily with an iCare rebound tonometer and averaged for 3 consecutive days to obtain baseline pressure as previously described (Roddy et al., Invest Ophthalmol Vis Sci. 58:2715-24(2017)). At this point, dexamethasone acetate suspension (200 μg in 20 μl volume) was injected weekly into the inferior conjunctival fornix of one eye in a slow release formulation (sodium chloride [0.667 g/100 mL], edetate disodium USP dehydrate [0.05 g/100 mL], sodium bisulfate [0.1 g/100 mL], and creatinine [0/5 g/100 mL], pH 7) as previously described (Patel et al., Am J Pathol. 187:713-23(2017). The fellow eye received a weekly injection with slow release formulation (vehicle) without the dexamethasone. IOP was obtained 48 and 72 hrs post-injection, averaged, and recorded as the weekly IOP. After a sustained and elevated IOP response was observed in the dexamethasone injected group (experimental weeks 1-3), mice were randomized into two groups and dexamethasone injections were continued weekly for the duration of the experiment: in Group 1, both eyes were treated once daily with topical PBS (5 n=8; experimental weeks 4-6, treatment weeks 1-3), and in Group 2, both eyes were treated once daily with topical STC-1 (Biovender, Asheville, NC, 5 μL; 0.5 n=10; experimental weeks 4-6, treatment weeks 1-3). In the final phase of the experiment (experimental weeks 7-9, treatment weeks 4-6), dexamethasone-injected animals in Group 2 continued to receive topical STC-1 while treatment was halted in the vehicle-injected fellow eye for a medication wash-out period (FIG. 22). For statistical purposes, the final week of each condition was selected (i.e. weeks 3, 6, and 9) for analysis. To determine if a difference in IOP was present among groups at week 6 and 9, a Kruskal-Wallis test was performed. An unpaired t-test was used to directly compare the two groups. Values for all statistical tests were considered significant at P<0.05.

For examination of STC-1 treatment in a chronic model of ocular hypertension and GON, DBA/2J mice aged 14 months (n=10) were selected. Since variability in IOP is high between eyes and among mice (Turner et al., Clin Exp Ophthalmol. 45:911-22(2017), for expression of data in a longitudinal fashion, IOP was expressed normalized to an average of the baseline IOPs. Utilizing mice without corneal calcification (Turner et al., Clin Exp Ophthalmol. 45:911-22(2017), baseline IOP measurements were obtained, and then one eye was treated once daily for 5 days with topical STC-1 (5 μL; 0.5 μg/μL) and the fellow eye received topical PBS (5 μL). For statistical purposes, at full treatment response, experimental days 6-8 (treatment days 3-5) were averaged and taken as a single value of IOP. A paired T-test was performed, and values were considered significant at P<0.05. For both models, a second laboratory member unfamiliar with the experimental design independently validated the IOP measurements at multiple time-points during the experiment.

For both models histologic analysis was performed. At the conclusion of each experiment, whole eyes were enucleated, fixed, processed, sectioned, stained with toluidine blue, and examined under a light microscope as previously described (Roddy et al., Invest Ophthalmol Vis Sci. 58:2715-24(2017)).

Results

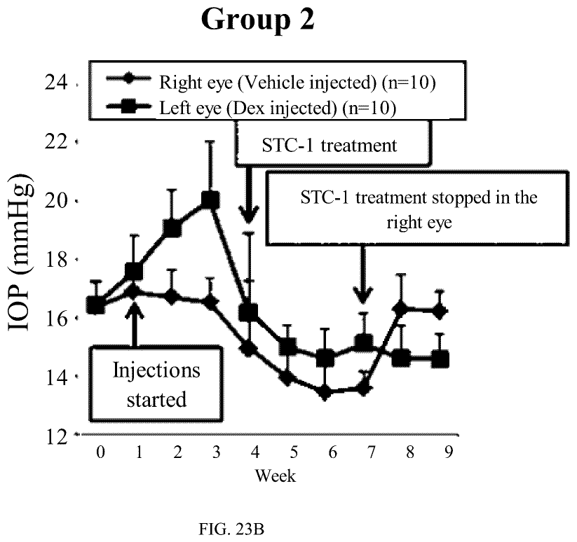

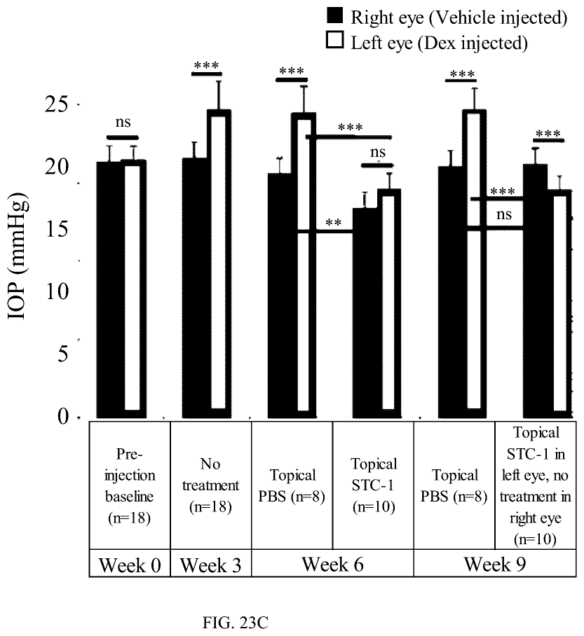

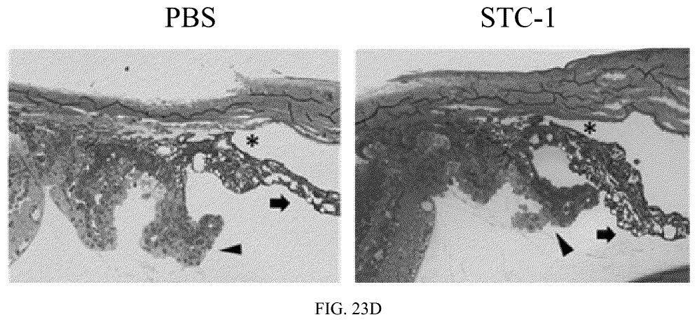

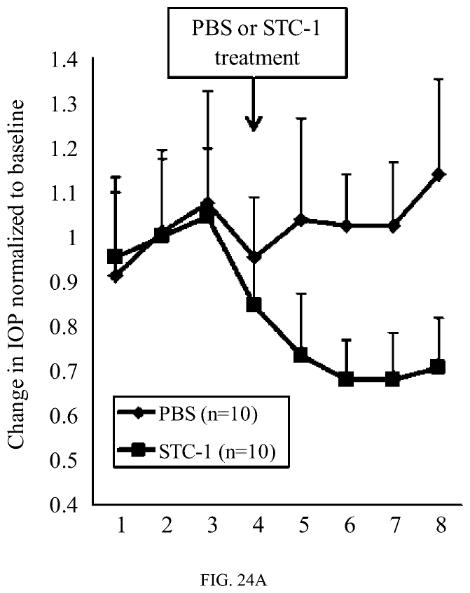

For the steroid-induced ocular hypertension model, prior to injection of dexamethasone, baseline IOP measurements were assessed and found to be similar between left and right eyes (16.1±1.1 vs 16.2±1.2 mmHg, P>0.8, n=18, FIG. 23A-23D). Following 3 weekly injections of dexamethasone, a significant increase in IOP in the dexamethasone-injected eyes compared to vehicle-injected eyes occurred (18%, 16.6±1.0 vs 19.7±1.8 mmHg, P<0.05, n=18, FIG. 23A-23C). This was maintained with weekly dexamethasone injections for 3 additional weeks comparing PBS-treated vehicle-injected mice (n=8, 15.6±1.1 mmHg) to PBS-treated dexamethasone-injected mice (n=8, 19.5±1.7 mmHg, P<0.001, 25% change, FIG. 23A). With the steroid-induced ocular hypertensive model established, treatment with topical STC-1 resulted in a 25% IOP reduction when compared to PBS-treatment of dexamethasone-injected eyes (19.5±1.7, n=8, vs 14.6±1.2 mmHg, n=10, P<0.001, FIG. 23B, week 6). This was maintained through 3 additional weeks of dexamethasone-injections and topical STC-1 treatments (26%, 19.7±1.3, n=8, vs 14.6±0.9 mmHg, n=10, P<0.001, FIG. 23B, week 9). When comparing STC-1-treated dexamethasone-injected eyes (n=10, FIG. 23B) to PBS-treated vehicle-injected eyes (n=8, FIG. 23A), there was no significant difference in IOP (6%, 14.6±1.0 vs 15.6±1.2 mm Hg, P=0.08, FIG. 23C) indicating that STC-1 treatment reduced IOP in the steroid-induced ocular hypertension eye to levels seen in normotensive mice.

In addition to STC-1 lowering IOP in steroid induced ocular hypertension eyes, STC-1 also reduced pressure in normotensive eyes as previously reported (Roddy et al., Invest Ophthalmol Vis Sci. 58:2715-24(2017); Roddy et al., PLoS ONE. (2020)). STC-1 treatment of vehicle-injected eyes (n=8) reduced IOP from 15.6±1.1 mmHg to 13.4±1.2 mmHg (14% decrease, P<0.005, FIG. 23B). Once STC-1 was washed out from the vehicle-injected eye, IOP returned to baseline levels (16.2±0.7, n=10, vs 16.1±1.7 mmHg, n=8, P>0.1, FIG. 23B). Interestingly, the IOP was even lower in the STC-1-treated dexamethasone-injected eyes when comparing to the untreated vehicle-injected eyes (10%, 16.2±0.7 vs 14.6±1.0 mmHg, P<0.001, FIG. 23C). Representative toluidine blue-stained eye sections of steroid-induced ocular hypertension mice were examined following treatment with PBS and STC-1 (FIG. 23D). In both treatment groups, normal-appearing open angles (asterisk), iris (arrow), and ciliary body (chevron) were observed.

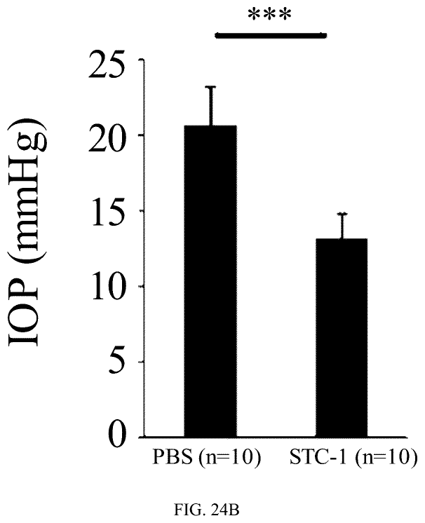

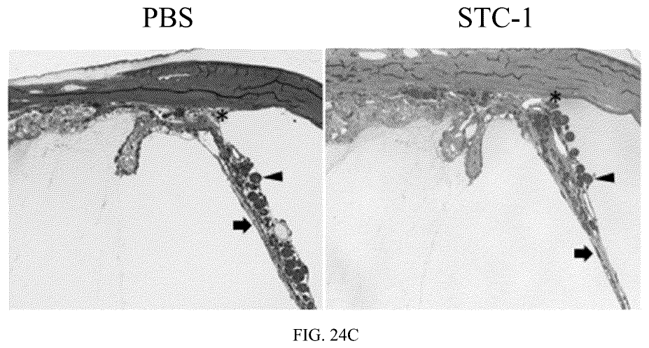

For DBA/2J mice, eyes treated with STC-1 showed a steady decrease in IOP until the treatment plateaued at treatment days 3-5 (FIG. 24A). No detectable change in IOP was observed in PBS treated eyes. Using the combined average IOP from treatment days 3-5 compared to baseline, STC-1 lowered IOP compared to vehicle control (37%, 20.7±2.6 mmHg vs 13.1±1.7 mm Hg, P<0.001, FIG. 24B). Representative toluidine blue-stained sections of 14-month-old DBA/2J mice treated with PBS in one eye and STC-1 in the fellow eye showed age-appropriate angle anatomy in this model (John et al., Invest Ophthalmol Vis Sci. 39:951-62(1998)) including angle closure with synechiae formation (asterisk), iris atrophy (arrow), and pigment-laden macrophages (chevron) (FIG. 24C). Though subtle variations in elements of the disease phenotype were observed in this chronic model, significant differences in angle morphology were not observed when comparing PBS-treated eyes with STC-1-treated eyes.

These results demonstrate that administration of STC-1 can successfully reduce IOP in both conditions of normal ocular tension and ocular hypertension.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treatment for reducing intraocular pressure (IOP) in an eye of a human patient having an ocular disorder, wherein the method comprises:
administering to the anterior chamber of the patient's eye a therapeutically effective amount of a recombinant adeno-associated virus (rAAV) comprising an AAV capsid and a nucleic acid encoding a stanniocalcin-1 (STC-1) polypeptide operably linked to a promoter.

2. The method of claim 1, wherein the rAAV is administered to the patient two or more times.

3. The method of claim 2, wherein the rAAV is administered at least 120 days apart.

4. The method of claim 2, wherein the rAAV is administered at least 1 year apart.

5. The method of claim 1, wherein the administration comprises intracameral administration.

6. The method of claim 1, wherein the STC-1 polypeptide comprises an amino acid sequence selected from SEQ ID NO: 2, SEQ ID NO: 4, SEQ ID NO: 6, SEQ ID NO: 14, or an amino acid sequence at least 95% identical thereto.

7. The method of claim 1, wherein the nucleic acid encoding STC-1 comprises a nucleic acid sequence selected from SEQ ID NO: 1, SEQ ID NO: 3, SEQ ID NO: 5, SEQ ID NO: 13, or a nucleic acid sequence at least 95% identical thereto.

8. The method of claim 1, wherein the promoter is a constitutively active promoter.

9. The method of claim 8, wherein the constitutively active promoter is selected from a chicken β-actin promoter, cytomegalovirus (CMV) promoter, a SV40 promoter, human β-actin promoter, a human elongation factor-1-alpha (hEF-1a) promoter, a phosphoglycerate kinase (PGK) promoter, or a ubiquitin C (UbiC) promoter.

10. The method of claim 8, wherein the constitutively active promoter is a chicken β-actin promoter derived from a nucleic acid sequence selected from the group consisting of the nucleic acid sequence of SEQ ID NO: 33, SEQ ID NO: 34, and SEQ ID NO: 35, or a nucleic acid sequence at least 80% identical thereto.

11. The method of claim 1, wherein the promoter is a cell specific promoter.

12. The method of claim 11, wherein the cell specific promoter is selected from human synapsin 1 (hSYN1) promoter, human rhodopsin (Rho) promoter, human rhodopsin kinase 1 (hRK1) promoter, human inter-photoreceptor retinoid binding protein/retinol-binding protein 3 (IRBP/hIRBP241) promoter, human red opsin (PR2.1/CHOP S2053) promoter, hIRBP enhancer fused to cone transducin alpha promoter (IRBP/GNAT2) promoter, human vitelliform macular dystrophy/bestrophin 1 (VMD2/BEST1) promoter, VE-cadherin/Cadherin 5 (CDH5)/CD144 promoter, Thy 1 promoter, neurofilament heavy chain (NEFH) promoter, retinal pigmented epithelium 65 (RPE65) promoter, Purkinje cell protein 2 (PCP2) promoter, G Protein Subunit Gamma Transducin 2 (GNGT2) promoter, Phosphodiesterase 6H (PDE6H) promoter, Paired Like Homeodomain 3 (PITX3) promoter, claudin 5 (CLDN5) promoter, Nuclear Receptor Subfamily 2 Group E Member 1 (NR2E1) promoter, paired box 6 (PAX6) promoter, 770En 454P(hGRM6), or fibroblast-specific protein 1 (FLP1/S100A4) promoter.

13. The method of claim 1, wherein the nucleic acid encoding a stanniocalcin-1 (STC-1) polypeptide operably linked to a promoter is selected from SEQ ID NO: 50, SEQ ID NO: 51, SEQ ID NO: 52, or SEQ ID NO:53, or a nucleic acid sequence at least 95% identical thereto.

14. The method of claim 1, wherein the AAV capsid is derived from an AAV2 capsid.

15. The method of claim 1, wherein the ocular disorder is selected from glaucomatous optic neuropathy (GON), ocular hypertension, glaucoma, primary glaucoma, open angle glaucoma angle-closure glaucoma, congenital glaucoma, secondary glaucoma, neovascular glaucoma, pigmentary glaucoma, exfoliation glaucoma, or uveitic glaucoma, normal-tension glaucoma (NTG), or radiation papillopathy.

16. The method of claim 15 wherein the ocular disorder is glaucomatous optic neuropathy (GON).

17. The method of claim 15, wherein the ocular disorder is radiation papillopathy.

18. A method of treatment for reducing intraocular pressure (IOP) in an eye of a patient in need thereof, wherein the method comprises administering to the eye of the patient a therapeutically effective amount of a recombinant adeno-associated virus (rAAV) comprising a AAV capsid and a nucleic acid encoding a stanniocalcin-1 (STC-1) polypeptide operably linked to a promoter, wherein the rAAV is administered into the subconjunctival space of the eye.

19. The method of claim 18, wherein the rAAV is administered to the patient two or more times.

20. A method of treatment for reducing intraocular pressure (IOP) in an eye of a patient in need thereof, wherein the method comprises administering to the eye of the patient a therapeutically effective amount of a recombinant adeno-associated virus (rAAV) comprising a AAV capsid and a nucleic acid encoding a stanniocalcin-1 (STC-1) polypeptide operably linked to a promoter, wherein the rAAV is administered into the subretinal space of the eye.

21. The method of claim 20, wherein the rAAV is administered to the patient two or more times.


SEQUENCE LISTING (57 sequences)

SEQ ID NO: 1 - DNA, 744 bp, Homo sapiens
  source 1..744: mol_type = genomic DNA
atgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataa

SEQ ID NO: 2 - protein, 247 aa, Homo sapiens
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 3 - DNA, 693 bp, Homo sapiens
  source 1..693: mol_type = genomic DNA
atgcatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataa

SEQ ID NO: 4 - protein, 230 aa, Homo sapiens
MHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 5 - DNA, 648 bp, Homo sapiens
  source 1..648: mol_type = genomic DNA
atggtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataa

SEQ ID NO: 6 - protein, 215 aa, Homo sapiens
MVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 7 - DNA, 771 bp, synthetic construct
  source 1..771: mol_type = other DNA
atgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcaagcgactacaaggacgacgatgacaagtaa

SEQ ID NO: 8 - protein, 256 aa, synthetic construct
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESASDYKDDDDK

SEQ ID NO: 9 - DNA, 678 bp, synthetic construct
  source 1..678: mol_type = other DNA
atggtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcagcaagcgactacaaggacgacgatgacaagtaa

SEQ ID NO: 10 - protein, 241 aa, synthetic construct
MTHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESAASDYKDDDDK

SEQ ID NO: 11 - DNA, 675 bp, synthetic construct
  source 1..675: mol_type = other DNA
atggtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcaagcgactacaaggacgacgatgacaagtaa

SEQ ID NO: 12 - protein, 224 aa, synthetic construct
MVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESASDYKDDDDK

SEQ ID NO: 13 - DNA, 537 bp, Homo sapiens
  source 1..537: mol_type = genomic DNA
atgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataa

SEQ ID NO: 14 - protein, 178 aa, Homo sapiens
MYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 15 - DNA, 744 bp, Mus musculus
  source 1..744: mol_type = genomic DNA
atgctccaaaactcagcagtgattctggcgctggtcatcagtgcagctgcagcgcacgaggcggaacaaaatgattctgtgagccccagaaaatcccgggtggcggctcaaaattcagctgaagtggttcgctgcctcaacagtgccctgcaggttggctgcggggcttttgcatgcctggaaaactccacatgtgacacagatgggatgtacgacatttgtaaatccttcttgtacagtgctgctaaatttgacactcagggaaaagcatttgtcaaagagagcttaaagtgcatcgccaatgggatcacctccaaggtattccttgccattcggaggtgttcgactttccagaggatgatcgccgaggtgcaggaggactgctacagcaagcttaacgtttgcagcatcgccaagcgcaacccggaagccatcactgaagtcatacagctgcccaatcacttctccaacagatactacaacagacttgtccgaagccttctggaatgtgatgaagacacggtcagtacaatcagagacagcctgatggagaagatcgggcccaacatggccagcctcttccacatcctgcagacagaccactgtgcccagacacaccccagagctgacttcaataggaggcgcacaaatgagccacagaagctgaaagtcctcctcaggaacctccgaggtgagggggactctccctcacacatcaaacgcacctcccaagagagtgcgtaa

SEQ ID NO: 16 - protein, 247 aa, Mus musculus
MLQNSAVILALVISAAAAHEAEQNDSVSPRKSRVAAQNSAEVVRCLNSALQVGCGAFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQESA

SEQ ID NO: 17 - DNA, 693 bp, Mus musculus
  source 1..693: mol_type = genomic DNA
atgcacgaggcggaacaaaatgattctgtgagccccagaaaatcccgggtggcggctcaaaattcagctgaagtggttcgctgcctcaacagtgccctgcaggttggctgcggggcttttgcatgcctggaaaactccacatgtgacacagatgggatgtacgacatttgtaaatccttcttgtacagtgctgctaaatttgacactcagggaaaagcatttgtcaaagagagcttaaagtgcatcgccaatgggatcacctccaaggtattccttgccattcggaggtgttcgactttccagaggatgatcgccgaggtgcaggaggactgctacagcaagcttaacgtttgcagcatcgccaagcgcaacccggaagccatcactgaagtcatacagctgcccaatcacttctccaacagatactacaacagacttgtccgaagccttctggaatgtgatgaagacacggtcagtacaatcagagacagcctgatggagaagatcgggcccaacatggccagcctcttccacatcctgcagacagaccactgtgcccagacacaccccagagctgacttcaataggaggcgcacaaatgagccacagaagctgaaagtcctcctcaggaacctccgaggtgagggggactctccctcacacatcaaacgcacctcccaagagagtgcgtaa

SEQ ID NO: 18 - protein, 230 aa, Mus musculus
MHEAEQNDSVSPRKSRVAAQNSAEVVRCLNSALQVGCGAFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQESA

SEQ ID NO: 19 - DNA, 648 bp, Mus musculus
  source 1..648: mol_type = genomic DNA
atggtggcggctcaaaattcagctgaagtggttcgctgcctcaacagtgccctgcaggttggctgcggggcttttgcatgcctggaaaactccacatgtgacacagatgggatgtacgacatttgtaaatccttcttgtacagtgctgctaaatttgacactcagggaaaagcatttgtcaaagagagcttaaagtgcatcgccaatgggatcacctccaaggtattccttgccattcggaggtgttcgactttccagaggatgatcgccgaggtgcaggaggactgctacagcaagcttaacgtttgcagcatcgccaagcgcaacccggaagccatcactgaagtcatacagctgcccaatcacttctccaacagatactacaacagacttgtccgaagccttctggaatgtgatgaagacacggtcagtacaatcagagacagcctgatggagaagatcgggcccaacatggccagcctcttccacatcctgcagacagaccactgtgcccagacacaccccagagctgacttcaataggaggcgcacaaatgagccacagaagctgaaagtcctcctcaggaacctccgaggtgagggggactctccctcacacatcaaacgcacctcccaagagagtgcgtaa

SEQ ID NO: 20 - protein, 215 aa, Mus musculus
MVAAQNSAEVVRCLNSALQVGCGAFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQESA

SEQ ID NO: 21 - protein, 246 aa, Mus musculus
MLQNSAVILALVISAAAAHEAEQNDSVSPRKSRVAAQNSAEVVRCLNSACRLAAGFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQESA

SEQ ID NO: 22 - protein, 229 aa, Mus musculus
MHEAEQNDSVSPRKSRVAAQNSAEVVRCLNSACRLAAGFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQESA

SEQ ID NO: 23 - protein, 214 aa, Mus musculus
MVAAQNSAEVVRCLNSACRLAAGFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQESA

SEQ ID NO: 24 - DNA, 744 bp, Rattus norvegicus
  source 1..744: mol_type = genomic DNA
atgctccaaaactcagcagtgattctggcgctggtcatcagtgctgctgcagctcacgaggcggaacagaatgattctgtgagccccagaaaatcccgggtggcggctcaaaattcagctgaagtggtccgctgcctcaacagtgccctacaggttggctgtggggcttttgcatgcctggaaaactccacatgtgacacagatgggatgtacgacatttgtaaatccttcttgtacagtgctgctaaatttgacactcagggaaaagcatttgtcaaagagagcttaaagtgcatcgccaatgggatcacctccaaggtcttccttgccattcggaggtgttctactttccagaggatgatcgccgaggtgcaggaggactgctacagcaagctcaatgtttgcagcattgccaagcgcaacccggaagccatcactgaagtcatacagctgcccaatcacttctccaacagatactacaacagacttgtccgaagccttctggaatgtgatgaagatacggtcagcacaatcagagacagcctgatggagaagatcgggcccaacatggccagcctcttccatatcctgcagacagaccactgtgcccagacacaccccagagctgacttcaataggaggcgcacaaatgagccacagaagctgaaagtcctcctcaggaacctccgaggtgagggggattctccctcacacatcaaacgcacctcccaagagaatgcgtaa

SEQ ID NO: 25 - protein, 247 aa, Rattus norvegicus [sequence omitted]

SEQ ID NO: 26 - DNA, 693 bp, Rattus norvegicus
  source 1..693: mol_type = genomic DNA
atgcacgaggcggaacagaatgattctgtgagccccagaaaatcccgggtggcggctcaaaattcagctgaagtggtccgctgcctcaacagtgccctacaggttggctgtggggcttttgcatgcctggaaaactccacatgtgacacagatgggatgtacgacatttgtaaatccttcttgtacagtgctgctaaatttgacactcagggaaaagcatttgtcaaagagagcttaaagtgcatcgccaatgggatcacctccaaggtcttccttgccattcggaggtgttctactttccagaggatgatcgccgaggtgcaggaggactgctacagcaagctcaatgtttgcagcattgccaagcgcaacccggaagccatcactgaagtcatacagctgcccaatcacttctccaacagatactacaacagacttgtccgaagccttctggaatgtgatgaagatacggtcagcacaatcagagacagcctgatggagaagatcgggcccaacatggccagcctcttccatatcctgcagacagaccactgtgcccagacacaccccagagctgacttcaataggaggcgcacaaatgagccacagaagctgaaagtcctcctcaggaacctccgaggtgagggggattctccctcacacatcaaacgcacctcccaagagaatgcgtaa

SEQ ID NO: 27 - protein, 230 aa, Rattus norvegicus
MHEAEQNDSVSPRKSRVAAQNSAEVVRCLNSALQVGCGAFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQENA

SEQ ID NO: 28 - DNA, 648 bp, Rattus norvegicus
  source 1..648: mol_type = genomic DNA
atggtggcggctcaaaattcagctgaagtggtccgctgcctcaacagtgccctacaggttggctgtggggcttttgcatgcctggaaaactccacatgtgacacagatgggatgtacgacatttgtaaatccttcttgtacagtgctgctaaatttgacactcagggaaaagcatttgtcaaagagagcttaaagtgcatcgccaatgggatcacctccaaggtcttccttgccattcggaggtgttctactttccagaggatgatcgccgaggtgcaggaggactgctacagcaagctcaatgtttgcagcattgccaagcgcaacccggaagccatcactgaagtcatacagctgcccaatcacttctccaacagatactacaacagacttgtccgaagccttctggaatgtgatgaagatacggtcagcacaatcagagacagcctgatggagaagatcgggcccaacatggccagcctcttccatatcctgcagacagaccactgtgcccagacacaccccagagctgacttcaataggaggcgcacaaatgagccacagaagctgaaagtcctcctcaggaacctccgaggtgagggggattctccctcacacatcaaacgcacctcccaagagaatgcgtaa

SEQ ID NO: 29 - protein, 215 aa, Rattus norvegicus
MVAAQNSAEVVRCLNSALQVGCGAFACLENSTCDTDGMYDICKSFLYSAAKFDTQGKAFVKESLKCIANGITSKVFLAIRRCSTFQRMIAEVQEDCYSKLNVCSIAKRNPEAITEVIQLPNHFSNRYYNRLVRSLLECDEDTVSTIRDSLMEKIGPNMASLFHILQTDHCAQTHPRADFNRRRTNEPQKLKVLLRNLRGEGDSPSHIKRTSQENA

SEQ ID NO: 30 - protein, 8 aa, synthetic construct
DYKDDDDK

SEQ ID NO: 31 - protein, 9 aa, synthetic construct
SDYKDDDDK

SEQ ID NO: 32 - protein, 10 aa, synthetic construct
ASDYKDDDDK

SEQ ID NO: 33 - DNA, 1718 bp, synthetic construct
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 34 - DNA, 976 bp, synthetic construct
  source 1..976: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggcggccgc

SEQ ID NO: 35 - DNA, 283 bp, synthetic construct
  source 1..283: mol_type = other DNA
catggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 36 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 37 - DNA, 344 bp, synthetic construct
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 38 - DNA, 1252 bp, synthetic construct
  source 1..1252: mol_type = other DNA
gttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcct

SEQ ID NO: 39 - DNA, 269 bp, synthetic construct
  source 1..269: mol_type = other DNA
caatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccag

SEQ ID NO: 40 - DNA, 511 bp, synthetic construct
  source 1..511: mol_type = other DNA
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 41 - DNA, 1177 bp, synthetic construct
  source 1..1177: mol_type = other DNA
ggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 42 - DNA, 490 bp, synthetic construct
  source 1..490: mol_type = other DNA
gatctaggcctactagtctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcga

SEQ ID NO: 43 - DNA, 236 bp, synthetic construct
  source 1..236: mol_type = other DNA
tcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 44 - DNA, 161 bp, synthetic construct
  source 1..161: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggt

SEQ ID NO: 45 - DNA, 143 bp, synthetic construct
  source 1..143: mol_type = other DNA
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggt

SEQ ID NO: 46 - DNA, 168 bp, synthetic construct
  source 1..168: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 47 - DNA, 142 bp, synthetic construct
  source 1..142: mol_type = other DNA
aacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 48 - DNA, 144 bp, synthetic construct
  source 1..144: mol_type = other DNA
aacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 49 - DNA, 167 bp, synthetic construct
  source 1..167: mol_type = other DNA
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 50 - DNA, 2024 bp, synthetic construct
  source 1..2024: mol_type = other DNA
  misc_feature 1..143: note = 5' ITR
  misc_feature 166..655: note = hSyn1 promoter
  misc_feature 686..827: note = SV40 SD/SA
  misc_feature 866..1606: note = hSTC-1
  misc_feature 1607..1630: note = FLAG-tag
  misc_feature 1635..1870: note = bGH Poly A
  misc_feature 1882..2024: note = 3' ITR
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccgatctaggcctactagtctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaattcactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcagactacaaggacgacgatgataagtaagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 51 - DNA, 2000 bp, synthetic construct
  source 1..2000: mol_type = other DNA
  misc_feature 1..143: note = 5' ITR
  misc_feature 166..655: note = hSyn1 promoter
  misc_feature 686..827: note = SV40 SD/SA
  misc_feature 866..1606: note = hSTC-1
  misc_feature 1611..1847: note = bGH Poly A
  misc_feature 1858..2000: note = 3' ITR
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccgatctaggcctactagtctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaattcactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 52 - DNA, 2300 bp, synthetic construct
  source 1..2300: mol_type = other DNA
  misc_feature 1..143: note = 5' ITR
  misc_feature 526..808: note = CBA promoter
  misc_feature 809..901: note = Exon 1
  misc_feature 902..1103: note = Chimeric intron
  misc_feature 1143..1883: note = hSTC-1
  misc_feature 1884..1907: note = FLAG-1
  misc_feature 1933..2147: note = bGH polyA
  misc_feature 2159..2300: note = 3' ITR
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggcggccgcatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcagactacaaggacgacgatgataagtaagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 53 - DNA, 2276 bp, synthetic construct
  source 1..2276: mol_type = other DNA
  misc_feature 1..143: note = 5' ITR
  misc_feature 526..808: note = CBA promoter
  misc_feature 809..901: note = Exon 1
  misc_feature 902..1103: note = Chimeric intron
  misc_feature 1143..1883: note = hSTC-1
  misc_feature 1892..2122: note = bGH polyA
  misc_feature 2135..2276: note = 3' ITR
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggcggccgcatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 54 - DNA, 292 bp, synthetic construct
  source 1..292: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggc

SEQ ID NO: 55 - DNA, 1317 bp, synthetic construct
  source 1..1317: mol_type = other DNA
ctacttctaaccctcactgggtttgtagcccaccctgagaggttgacccgaattataactcccctatttcatgccatttcacctctaactctccaccccaacctggattcttcattcctgacactcatcccaactttaaatggcccctcctgataccctctccgaacctgagatctatccgtgagcccccacgcctcactgccactccactccatcactacctcacccaggacctttcccactgacgttcctgaggtggtcccagagcctcctttgggtgtgagcctgttcccctccagatccccccgccccgaccctgagccttacttggcatggcagacagtaccgggcatggggatccccaccccagtttttgtttctgaatctttatttttttaagagacaaggtcctctgtgttgctcaggctggagagcagtggcttgagcatagccaactgcagtctcgaactcctgggctcaaatgatcctcctgtctcagcttcctgactagctgggactacaggctacagccatgctgcccagctaattaaaaaaaaaaattgtttttcctttttatagagacagaagtctctctatgttgcctaggctggtcttgaactcctggcctcaggcgatcctcccatctcccccctagcttttgtgtcaccacatttccagggcaatctcccacctgtcacccaccaccccctgcatctcctttcctaggtccccatgggactactccctgtcccccatgctccaggcacaggctgccccttcctccacctctctaaaactcaggctgagctatgtacactgggtggtgcccatctcatccagtcccctgctagtaaccgctagggcttacccgttacccacgggtgcccacctgggaacaggaggcttggttccacggctgggctggtggagggtgctgtggcacttaccgcatcagcccacagcaggaaggcagtatccgctctcccctgtcccctgctatgggcagggcctggctggggtataaataggtcagacctctgggccgtccccattcttcccctctctacaaccctctctcctcagcgcttcttctttcttggtttggtgagttgtgttggcctgactggcatgcaaggggtgtcagaggccagggctggggaaggagaaggggaggctggtgggggccagatgtgctaaagagatccagatgtgagattctgatgtggaactctgggtggattgtgtgcgtgggtgtgcatggcacacacacacatgcacgtaagacggaggaaaaaacaaacagaaaagtgagcaagt

SEQ ID NO: 56 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 57 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa